SEQUENCE LISTING (230 sequences)

SEQ ID NO: 1 - DNA, 54 bp, synthetic construct
  source 1..54: mol_type = other DNA
  modified_base 1: mod_base = OTHER; note = 5' 6-Carboxyfluorescein modified nucleotide
gatcacttagagcaatcggcccagactgagcacgtgatggcagagtactaggat

SEQ ID NO: 2 - DNA, 54 bp, synthetic construct
  source 1..54: mol_type = other DNA
atcctagtactctgccatcacgtgctcagtctgggccgattgctctaagtgatc

SEQ ID NO: 3 - DNA, 58 bp, synthetic construct
  source 1..58: mol_type = other DNA
ggatcctaatacgactcactataggcccagactgagcacgtgagttttagagctagaa

SEQ ID NO: 4 - DNA, 76 bp, synthetic construct
  source 1..76: mol_type = other DNA
gcaccgactcggtgccactttttcaagttgataacggactagccttattttaacttgctatttctagctctaaaac

SEQ ID NO: 5 - DNA, 24 bp, synthetic construct
  source 1..24: mol_type = other DNA
ggatcctaatacgactcactatag

SEQ ID NO: 6 - DNA, 16 bp, synthetic construct
  source 1..16: mol_type = other DNA
aaaagcaccgactcgg

SEQ ID NO: 7 - DNA, 38 bp, synthetic construct
  source 1..38: mol_type = other DNA
aaaagagcacgagattgcagagtactgcaccgactcgg

SEQ ID NO: 8 - DNA, 41 bp, synthetic construct
  source 1..41: mol_type = other DNA
aaaaactgagcacgagattgcagagtactgcaccgactcgg

SEQ ID NO: 9 - DNA, 48 bp, synthetic construct
  source 1..48: mol_type = other DNA
aaaagagcacgagattgcagagtactagatgacgtagcaccgactcgg

SEQ ID NO: 10 - DNA, 51 bp, synthetic construct
  source 1..51: mol_type = other DNA
aaaaactgagcacgagattgcagagtactagatgacgtagcaccgactcgg

SEQ ID NO: 11 - DNA, 15 bp, synthetic construct
  source 1..15: mol_type = other DNA
  modified_base 1: mod_base = OTHER; note = 5' phosphate modified nucleotide
agattgcagagtact

SEQ ID NO: 12 - DNA, 25 bp, synthetic construct
  source 1..25: mol_type = other DNA
  modified_base 1: mod_base = OTHER; note = 5' phosphate modified nucleotide
agattgcagagtactagatgacgta

SEQ ID NO: 13 - DNA, 30 bp, synthetic construct
  source 1..30: mol_type = other DNA
  modified_base 1: mod_base = OTHER; note = 5' phosphate modified nucleotide
agattgcagagtactagatgacgtacgcag

SEQ ID NO: 14 - DNA, 50 bp, synthetic construct
  source 1..50: mol_type = other DNA
  modified_base 1: mod_base = OTHER; note = 5' phosphate modified nucleotide
agattgcagagtactagatgacgtacgcagaccaagaaccgcaagatgcg

SEQ ID NO: 15 - DNA, 70 bp, synthetic construct
  source 1..70: mol_type = other DNA
  modified_base 1: mod_base = OTHER; note = 5' phosphate modified nucleotide
agattgcagagtactagatgacgtacgcagaccaagaaccgcaagatgcgacggtgtacaagtaattgtc

SEQ ID NO: 16 - DNA, 90 bp, synthetic construct
  source 1..90: mol_type = other DNA
  modified_base 1: mod_base = OTHER; note = 5' phosphate modified nucleotide
agattgcagagtactagatgacgtacgcagaccaagaaccgcaagatgcgacggtgtacaagtaattgtcaacagaccatcgtgttttca

SEQ ID NO: 17 - DNA, 24 bp, synthetic construct
  source 1..24: mol_type = other DNA
agtactctgcaatctcgtgcattt

SEQ ID NO: 18 - DNA, 25 bp, synthetic construct
  source 1..25: mol_type = other DNA
agtactctgcaatctcgtgcttttt

SEQ ID NO: 19 - DNA, 26 bp, synthetic construct
  source 1..26: mol_type = other DNA
agtactctgcaatctcgtgctctttt

SEQ ID NO: 20 - DNA, 27 bp, synthetic construct
  source 1..27: mol_type = other DNA
agtactctgcaatctcgtgctcatttt

SEQ ID NO: 21 - DNA, 28 bp, synthetic construct
  source 1..28: mol_type = other DNA
agtactctgcaatctcgtgctcagattt

SEQ ID NO: 22 - DNA, 29 bp, synthetic construct
  source 1..29: mol_type = other DNA
agtactctgcaatctcgtgctcagttttt

SEQ ID NO: 23 - DNA, 39 bp, synthetic construct
  source 1..39: mol_type = other DNA
ctgcgtacgtcatctagtactctgcaatctcgtgcattt

SEQ ID NO: 24 - DNA, 59 bp, synthetic construct
  source 1..59: mol_type = other DNA
cgcatcttgcggttcttggtctgcgtacgtcatctagtactctgcaatctcgtgcattt

SEQ ID NO: 25 - DNA, 41 bp, synthetic construct
  source 1..41: mol_type = other DNA
ctgcgtacgtcatctagtactctgcaatctcgtgctctttt

SEQ ID NO: 26 - DNA, 60 bp, synthetic construct
  source 1..60: mol_type = other DNA
cgcatcttgcggttcttggtctgcgtacgtcatctagtactctgcaatctcgtgctcttt

SEQ ID NO: 27 - DNA, 26 bp, synthetic construct
  source 1..26: mol_type = other DNA
  misc_feature 1..20: note = DNA
  misc_feature 21..22: note = RNA
  misc_feature 23..26: note = DNA
agtactctgcaatctcgtgctctttt

SEQ ID NO: 28 - DNA, 26 bp, synthetic construct
  source 1..26: mol_type = other DNA
  misc_feature 1..19: note = DNA
  misc_feature 20..22: note = RNA
  misc_feature 23..26: note = DNA
agtactctgcaatctcgtgctctttt

SEQ ID NO: 29 - DNA, 26 bp, synthetic construct
  source 1..26: mol_type = other DNA
  misc_feature 1..18: note = DNA
  misc_feature 19..22: note = RNA
  misc_feature 23..26: note = DNA
agtactctgcaatctcgtgctctttt

SEQ ID NO: 30 - DNA, 26 bp, synthetic construct
  source 1..26: mol_type = other DNA
  misc_feature 1..17: note = DNA
  misc_feature 18..22: note = RNA
  misc_feature 23..26: note = DNA
agtactctgcaatctcgtgctctttt

SEQ ID NO: 31 - DNA, 26 bp, synthetic construct
  source 1..26: mol_type = other DNA
  misc_feature 1..16: note = DNA
  misc_feature 17..22: note = RNA
  misc_feature 23..26: note = DNA
agtactctgcaatctcgtgctctttt

SEQ ID NO: 32 - DNA, 34 bp, synthetic construct
  source 1..34: mol_type = other DNA
  modified_base 1: mod_base = OTHER; note = 5' 6-Carboxyfluorescein modified nucleotide
gatcacttagagcaatcggcccagactgagcacg

SEQ ID NO: 33 - DNA, 20 bp, synthetic construct
  source 1..20: mol_type = other DNA
tgatggcagagtactaggat

SEQ ID NO: 34 - DNA, 42 bp, synthetic construct
  source 1..42: mol_type = other DNA
ctgcgtacgtcatctagtactctgcaatctcgtgctcatttt

SEQ ID NO: 35 - DNA, 43 bp, synthetic construct
  source 1..43: mol_type = other DNA
ctgcgtacgtcatctagtactctgcaatctcgtgctcagattt

SEQ ID NO: 36 - DNA, 44 bp, synthetic construct
  source 1..44: mol_type = other DNA
ctgcgtacgtcatctagtactctgcaatctcgtgctcagttttt

SEQ ID NO: 37 - DNA, 45 bp, synthetic construct
  source 1..45: mol_type = other DNA
ctgcgtacgtcatctagtactctgcaatctcgtgctcagtcattt

SEQ ID NO: 38 - DNA, 46 bp, synthetic construct
  source 1..46: mol_type = other DNA
ctgcgtacgtcatctagtactctgcaatctcgtgctcagtcttttt

SEQ ID NO: 39 - DNA, 47 bp, synthetic construct
  source 1..47: mol_type = other DNA
ctgcgtacgtcatctagtactctgcaatctcgtgctcagtctgtttt

SEQ ID NO: 40 - DNA, 15 bp, synthetic construct
  source 1..15: mol_type = other DNA
  modified_base 1: mod_base = OTHER; note = 5' phosphate modified nucleotide
ggatcgcagaggaaa

SEQ ID NO: 41 - DNA, 30 bp, synthetic construct
  source 1..30: mol_type = other DNA
  modified_base 1: mod_base = OTHER; note = 5' phosphate modified nucleotide
ggatcgcagaggaaaggaagccctgcttcc

SEQ ID NO: 42 - DNA, 26 bp, synthetic construct
  source 1..26: mol_type = other DNA
  misc_feature 1..15: note = DNA
  misc_feature 16..22: note = RNA
  misc_feature 23..26: note = DNA
agtactctgcaatctcgtgctctttt

SEQ ID NO: 43 - DNA, 26 bp, synthetic construct
  source 1..26: mol_type = other DNA
  misc_feature 1..21: note = DNA
  misc_feature 22: note = RNA
  misc_feature 23..26: note = DNA
agtactctgcaatctcgtgctctttt

SEQ ID NO: 44 - DNA, 26 bp, synthetic construct
  source 1..26: mol_type = other DNA
agtactctgcaatctcgtgctctttt

SEQ ID NO: 45 - DNA, 117 bp, synthetic construct
  source 1..117: mol_type = other DNA
  misc_feature 1..96: note = RNA
  misc_feature 97..116: note = DNA
  misc_feature 117: note = RNA
ggcccagactgagcacgtgagttttagagctagaaatagcaagttaaaataaggctagtccgttatcaacttgaaaaagtggcaccgagtcggtgctttcctctgcgatcccgtgct

SEQ ID NO: 46 - DNA, 118 bp, synthetic construct
  source 1..118: mol_type = other DNA
  misc_feature 1..96: note = RNA
  misc_feature 97..117: note = DNA
  misc_feature 118: note = RNA
ggcccagactgagcacgtgagttttagagctagaaatagcaagttaaaataaggctagtccgttatcaacttgaaaaagtggcaccgagtcggtgctttcctctgcgatcccgtgctt

SEQ ID NO: 47 - DNA, 119 bp, synthetic construct
  source 1..119: mol_type = other DNA
  misc_feature 1..96: note = RNA
  misc_feature 97..118: note = DNA
  misc_feature 119: note = RNA
ggcccagactgagcacgtgagttttagagctagaaatagcaagttaaaataaggctagtccgttatcaacttgaaaaagtggcaccgagtcggtgctttcctctgcgatcccgtgctct

SEQ ID NO: 48 - DNA, 119 bp, synthetic construct; identical to SEQ ID NO: 47
  source 1..119: mol_type = other DNA
  misc_feature 1..96: note = RNA
  misc_feature 97..116: note = DNA
  misc_feature 117..119: note = RNA

SEQ ID NO: 49 - DNA, 119 bp, synthetic construct; identical to SEQ ID NO: 47
  source 1..119: mol_type = other DNA
  misc_feature 1..96: note = RNA
  misc_feature 97..115: note = DNA
  misc_feature 116..119: note = RNA

SEQ ID NO: 50 - DNA, 119 bp, synthetic construct; identical to SEQ ID NO: 47
  source 1..119: mol_type = other DNA
  misc_feature 1..96: note = RNA
  misc_feature 97..114: note = DNA
  misc_feature 115..119: note = RNA

SEQ ID NO: 51 - DNA, 119 bp, synthetic construct; identical to SEQ ID NO: 47
  source 1..119: mol_type = other DNA
  misc_feature 1..96: note = RNA
  misc_feature 97..113: note = DNA
  misc_feature 114..119: note = RNA

SEQ ID NO: 52 - DNA, 119 bp, synthetic construct; identical to SEQ ID NO: 47
  source 1..119: mol_type = other DNA
  misc_feature 1..96: note = RNA
  misc_feature 97..112: note = DNA
  misc_feature 113..119: note = RNA

SEQ ID NO: 53 - DNA, 119 bp, synthetic construct; identical to SEQ ID NO: 47
  source 1..119: mol_type = other DNA
  misc_feature 1..96: note = RNA
  misc_feature 97..111: note = DNA
  misc_feature 112..119: note = RNA

SEQ ID NO: 54 - DNA, 134 bp, synthetic construct
  source 1..134: mol_type = other DNA
  misc_feature 1..96: note = RNA
  misc_feature 97..126: note = DNA
  misc_feature 127..134: note = RNA
ggcccagactgagcacgtgagttttagagctagaaatagcaagttaaaataaggctagtccgttatcaacttgaaaaagtggcaccgagtcggtgcggaagcagggcttcctttcctctgcgatcccgtgctct

SEQ ID NO: 55 - RNA, 119 nt, synthetic construct
  source 1..119: mol_type = other RNA
ggcccagactgagcacgtgagttttagagctagaaatagcaagttaaaataaggctagtccgttatcaacttgaaaaagtggcaccgagtcggtgctttcctctgcgatcccgtgctct

SEQ ID NO: 56 - DNA, 117 bp, synthetic construct
  source 1..117: mol_type = other DNA
  misc_feature 1..96: note = RNA
  misc_feature 97..106: note = DNA
  misc_feature 107..117: note = RNA
ggcccagactgagcacgtgagttttagagctagaaatagcaagttaaaataaggctagtccgttatcaacttgaaaaagtggcaccgagtcggtgctctgcgatcccgtgctctttt

SEQ ID NO: 57 - DNA, 117 bp, synthetic construct; identical to SEQ ID NO: 56
  source 1..117: mol_type = other DNA
  misc_feature 1..102: note = RNA
  misc_feature 103..110: note = DNA
  misc_feature 111..117: note = RNA

SEQ ID NO: 58 - protein, 298 aa, Paramecium bursaria chlorella virus 1
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDR

SEQ ID NO: 59 - protein, 298 aa, Paramecium bursaria chlorella virus 1
MTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEEDC

SEQ ID NO: 60 - protein, 346 aa, unidentified
MNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKM

SEQ ID NO: 61 - protein, 487 aa, unidentified
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 62 - protein, 359 aa, unidentified
MMNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKM

SEQ ID NO: 63 - protein, 671 aa, Escherichia coli
MESIEQQLTELRTTLRHHEYLYHVMDAPEIPDAEYDRLMRELRELETKHPELITPDSPTQRVGAAPLAAFSQIRHEVPMLSLDNVFDEESFLAFNKRVQDRLKNNEKVTWCCELKLDGLAVSILYENGVLVSAATRGDGTTGEDITSNVRTIRAIPLKLHGENIPARLEVRGEVFLPQAGFEKINEDARRTGGKVFANPRNAAAGSLRQLDPRITAKRPLTFFCYGVGVLEGGELPDTHLGRLLQFKKWGLPVSDRVTLCESAEEVLAFYHKVEEDRPTLGFDIDGVVIKVNSLAQQEQLGFVARAPRWAVAFKFPAQEQMTFVRDVEFQVGRTGAITPVARLEPVHVAGVLVSNATLHNADEIERLGLRIGDKVVIRRAGDVIPQVVNVVLSERPEDTREVVFPTHCPVCGSDVERVEGEAVARCTGGLICGAQRKESLKHFVSRRAMDVDGMGDKIIDQLVEKEYVHTPADLFKLTAGKLTGLERMGPKSAQNVVNALEKAKETTFARFLYALGIREVGEATAAGLAAYFGTLEALEAASIEELQKVPDVGIVVASHVHNFFAEESNRNVISELLAEGVHWPAPIVINAEEIDSPFAGKTVVLTGSLSQMSRDDAKARLVELGAKVAGSVSKKTDLVIAGEAAGSKLAKAQELGIEVIDEAEMLRLLGS

SEQ ID NO: 64 - protein, 268 aa, Haemophilus influenzae
MKFYRTLLLFFASSFAFANSDLMLLHTYNNQPIEGWVMSEKLDGVRGYWNGKQLLTRQGQRLSPPAYFIKDFPPFAIDGELFSERNHFEEISTITKSFKGDGWEKLKLYVFDVPDAEGNLFERLAKLKAHLLEHPTTYIEIIEQIPVKDKTHLYQFLAQVENLQGEGVVVRNPNAPYERKRSSQILKLKTARGEECTVIAHHKGKGQFENVMGALTCKNHRGEFKIGSGFNLNERENPPPIGSVITYKYRGITNSGKPRFATYWREKK

SEQ ID NO: 65 - protein, 911 aa, Homo sapiens
MAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDEPQEKKRKAAPKMKKVIGIIEHLKAPNLTNVNKISNIFEDVEFCVMSGTDSQPKPDLENRIAEFGGYIVQNPGPDTYCVIAGSENIRVKNIILSNKHDVVKPAWLLECFKTKSFVPWQPRFMIHMCPSTKEHFAREYDCYGDSYFIDTDLNQLKEVFSGIKNSNEQTPEEMASLIADLEYRYSWDCSPLSMFRRHTVYLDSYAVINDLSTKNEGTRLAIKALELRFHGAKVVSCLAEGVSHVIIGEDHSRVADFKAFRRTFKRKFKILKESWVTDSIDKCELQEENQYLI

SEQ ID NO: 66 - protein, 919 aa, Homo sapiens
MQRSIMSFFHPKKEGKAKKPEKEASNSSRETEPPPKAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 67 - protein, 1166 aa, Shigella flexneri
MKAGEESVAQVSGVREQAILTQAIRSELKTQGVLGHPEVTMTALSPVWLDSRSRYLRDMYRPGMVMEQWNPETRSHDRYVTERVTAQSHSLTLRNAQGETQVVRISSLDSSWSLFRPEKMPVADGERLRVTGKIPGLRVSGGDRLQVASVSEDAMTVVVPGRAEPATLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFADLGGEINAQIKRGDLLYVDVAKGYGTGLLVSRASYEAEKSILRHILEGKEAVTPLMERVPGELMEKLTSGQRAATRMILETSDRFTVVQGYAGVGKTTQFRAVMSAVNMLPESERPRVVGLGPTHRAVGEMRSAGVDAQTLASFLHDTQLQQRSGETPDFSNTLFLLDESSMVGNTDMARAYALIAAGGGRAVASGDTDQLQAIAPGQPFRLQQTRSAADVVIMKEIVRQTPELREAVYSLINRDVERALSGLERVKPSQVPRLEGAWAPEHSVTEFSHSQEAKLAEAQQKAMLKGEAFPDVPMTLYEAIVRDYTGRTPEAREQTLIVTHLNEDRRVLNSMIHDAREKAGELGKVQVMVPVLNTANIRDGELRRLSTWENNPDALALVDNVYHRIAGISKDDGLITLQDAEGNTRLISPREAVAEGVTLYTPDTIRVGTGDRIRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVFEPKPDREVMNAERLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTEQAVREIAGQERDRAAITEREAALPESVLREPQRVREAVREVARENLLQERLQQMERDMVRDLQKEKTPGGD

SEQ ID NO: 68 - protein, 351 aa, Nostoc sp.
MVSTLDDTKRNAIAEKLADAKLLQELIIENQERFLRESTDNEISNRIRDFLEDDRKNLGIIETVIVQYGIQKEPRQTVREMVDQVRQLMQGSQLNFFEKVAQHELLKHKQVMSGLLVHKAAQKVGADVLAAIGPLNTVNFENRAHQEQLKGILEILGVRELTGQDADQGIWGRVQDAIAAFSGAVGSAVTQGSDKQDMNIQDVIRMDHNKVNILFTELQQSNDPQKIQEYFGQIYKDLTAHAEAEEEVLYPRVRSFYGEGDTQELYDEQSEMKRLLEQIKAISPSAPEFKDRVRQLADIVMDHVRQEESTLFAAIRNNLSSEQTEQWATEFKAAKSKIQQRLGGQATGAGV

SEQ ID NO: 69 - protein, 1368 aa, Streptococcus pyogenes [sequence omitted]

SEQ ID NO: 70 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 71 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 72 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 73 - protein, 1629 aa, Francisella sp.
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDAIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 74 - protein, 7 aa, Simian virus 40
PKKKRKV

SEQ ID NO: 75 - protein, 17 aa, unidentified
KRTADGSEFEPKKKRKV

SEQ ID NO: 76 - protein, 16 aa, unidentified
KRPAATKKAGQAKKKK

SEQ ID NO: 77 - protein, 3000 aa, synthetic construct
  VARIANT 1..3000: note = This sequence may encompass 1-1000 GGS repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 78 - protein, 5000 aa, synthetic construct
  VARIANT 1..5000: note = This sequence may encompass 1-1000 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 79 - protein, 5000 aa, synthetic construct
  VARIANT 1..5000: note = This sequence may encompass 1-1000 EAAAK repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 80 - protein, 7 aa, synthetic construct
MSRPDPA

SEQ ID NO: 81 - protein, 10 aa, synthetic construct
MKIIEQLPSA

SEQ ID NO: 82 - protein, 10 aa, synthetic construct
VRHKLKRVGS

SEQ ID NO: 83 - protein, 12 aa, synthetic construct
SIVAQLSRPDPA

SEQ ID NO: 84 - protein, 12 aa, synthetic construct
GHGTGSTGSGSS

SEQ ID NO: 85 - protein, 12 aa, synthetic construct
GSAGSAAGSGEF

SEQ ID NO: 86 - protein, 15 aa, synthetic construct
VPFLLEPDNINGKTC

SEQ ID NO: 87 - protein, 16 aa, synthetic construct
SGSETPGTSESATPES

SEQ ID NO: 88 - protein, 33 aa, synthetic construct
SGGSSGGSSGSETPGTSESATPESSGGSSGGSS

SEQ ID NO: 89 - protein, 1737 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDRSGGSKRTADGSEFEPKKKRKV

SEQ ID NO: 90 - protein, 1737 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEEDCSGGSKRTADGSEFEPKKKRKV

SEQ ID NO: 91 - protein, 1785 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKMSGGSKRTADGSEFEPKKKRKV

SEQ ID NO: 92 - protein, 1926 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADGSEFEPKKKRKV

SEQ ID NO: 93 - protein, 1798 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSMNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKMSGGSKRTADGSEFEPKKKRKV

SEQ ID NO: 94 - protein, 2110 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSESIEQQLTELRTTLRHHEYLYHVMDAPEIPDAEYDRLMRELRELETKHPELITPDSPTQRVGAAPLAAFSQIRHEVPMLSLDNVFDEESFLAFNKRVQDRLKNNEKVTWCCELKLDGLAVSILYENGVLVSAATRGDGTTGEDITSNVRTIRAIPLKLHGENIPARLEVRGEVFLPQAGFEKINEDARRTGGKVFANPRNAAAGSLRQLDPRITAKRPLTFFCYGVGVLEGGELPDTHLGRLLQFKKWGLPVSDRVTLCESAEEVLAFYHKVEEDRPTLGFDIDGVVIKVNSLAQQEQLGFVARAPRWAVAFKFPAQEQMTFVRDVEFQVGRTGAITPVARLEPVHVAGVLVSNATLHNADEIERLGLRIGDKVVIRRAGDVIPQVVNVVLSERPEDTREVVFPTHCPVCGSDVERVEGEAVARCTGGLICGAQRKESLKHFVSRRAMDVDGMGDKIIDQLVEKEYVHTPADLFKLTAGKLTGLERMGPKSAQNVVNALEKAKETTFARFLYALGIREVGEATAAGLAAYFGTLEALEAASIEELQKVPDVGIVVASHVHNFFAEESNRNVISELLAEGVHWPAPIVINAEEIDSPFAGKTVVLTGSLSQMSRDDAKARLVELGAKVAGSVSKKTDLVIAGEAAGSKLAKAQELGIEVIDEAEMLRLLGSSGGSKRTADGSEFEPKKKRKV

SEQ ID NO: 95 - protein, 1707 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKFYRTLLLFFASSFAFANSDLMLLHTYNNQPIEGWVMSEKLDGVRGYWNGKQLLTRQGQRLSPPAYFIKDFPPFAIDGELFSERNHFEEISTITKSFKGDGWEKLKLYVFDVPDAEGNLFERLAKLKAHLLEHPTTYIEIIEQIPVKDKTHLYQFLAQVENLQGEGVVVRNPNAPYERKRSSQILKLKTARGEECTVIAHHKGKGQFENVMGALTCKNHRGEFKIGSGFNLNERENPPPIGSVITYKYRGITNSGKPRFATYWREKKSGGSKRTADGSEFEPKKKRKV

SEQ ID NO: 96 - protein, 2350 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDEPQEKKRKAAPKMKKVIGIIEHLKAPNLTNVNKISNIFEDVEFCVMSGTDSQPKPDLENRIAEFGGYIVQNPGPDTYCVIAGSENIRVKNIILSNKHDVVKPAWLLECFKTKSFVPWQPRFMIHMCPSTKEHFAREYDCYGDSYFIDTDLNQLKEVFSGIKNSNEQTPEEMASLIADLEYRYSWDCSPLSMFRRHTVYLDSYAVINDLSTKNEGTRLAIKALELRFHGAKVVSCLAEGVSHVIIGEDHSRVADFKAFRRTFKRKFKILKESWVTDSIDKCELQEENQYLISGGSKRTADGSEFEPKKKRKV

SEQ ID NO: 97 - protein, 2358 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSQRSIMSFFHPKKEGKAKKPEKEASNSSRETEPPPKAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTYSGGSKRTADGSEFEPKKKRKV

SEQ ID NO: 98 - protein, 1751 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDRSGGSKRTADGSEFESPKKKRKVGSGPAAKRVKLD

SEQ ID NO: 99 - protein, 1940 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADGSEFESPKKKRKVGSGPAAKRVKLD

SEQ ID NO: 100 - protein, 2364 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDEPQEKKRKAAPKMKKVIGIIEHLKAPNLTNVNKISNIFEDVEFCVMSGTDSQPKPDLENRIAEFGGYIVQNPGPDTYCVIAGSENIRVKNIILSNKHDVVKPAWLLECFKTKSFVPWQPRFMIHMCPSTKEHFAREYDCYGDSYFIDTDLNQLKEVFSGIKNSNEQTPEEMASLIADLEYRYSWDCSPLSMFRRHTVYLDSYAVINDLSTKNEGTRLAIKALELRFHGAKVVSCLAEGVSHVIIGEDHSRVADFKAFRRTFKRKFKILKESWVTDSIDKCELQEENQYLISGGSKRTADGSEFESPKKKRKVGSGPAAKRVKLD

SEQ ID NO: 101 - protein, 2074 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDSGGSKRTADGSEFESPKKKRKVGSGPAAKRVKLD

SEQ ID NO: 102 - RNA, 115 nt, synthetic construct
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide
gcgactcctggaagtggccagttttagagctagaaatagcaagttaaaataaggctagtccgttatcgacttgaaaaagtcggaccgagtcggtccagctgcggtattgtggcgt

SEQ ID NO: 103 - RNA, 115 nt, synthetic construct
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide
gctggccccccaccgccccagttttagagctagaaatagcaagttaaaataaggctagtccgttatcgacttgaaaaagtcggaccgagtcggtccgtggttccgggctgcatga

SEQ ID NO: 104 - RNA, 115 nt, synthetic construct
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide
ggaatcccttctgcagcaccgttttagagctagaaatagcaagttaaaataaggctagtccgttatcgacttgaaaaagtcggaccgagtcggtccagctgcggtattgtggcgt

SEQ ID NO: 105 - RNA, 115 nt, synthetic construct
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide
ggggtcccaggtgctgacgtgttttagagctagaaatagcaagttaaaataaggctagtccgttatcgacttgaaaaagtcggaccgagtcggtccgtggttccgggctgcatga

SEQ ID NO: 106 - RNA, 115 nt, synthetic construct
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide
ggcccagactgagcacgtgagttttagagctagaaatagcaagttaaaataaggctagtccgttatcgacttgaaaaagtcggaccgagtcggtccagctgcggtattgtggcgt

SEQ ID NO: 107 - RNA, 115 nt, synthetic construct
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide
gtcaaccagtatcccggtgcgttttagagctagaaatagcaagttaaaataaggctagtccgttatcgacttgaaaaagtcggaccgagtcggtccgtggttccgggctgcatga

SEQ ID NO: 108 - DNA, 154 bp, synthetic construct
  source 1..154: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked thymine
  modified_base 138: mod_base = OTHER; note = Locked nucleic acid
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139: note = DNA
  misc_feature 140..154: note = RNA
gcgactcctggaagtggccagttttagagctagaaatagcaagttaaaataaggctagtccgttatcgacttgaaaaagtcggaccgagtcggtccagctgcggtattgtggcgtggcggtctccgtcgtcaggatcatccacttccaggtttt

SEQ ID NO: 109 - DNA, 154 bp, synthetic construct; identical to SEQ ID NO: 108
  source 1..154: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked thymine
  modified_base 138: mod_base = OTHER; note = Locked nucleic acid
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139..142: note = DNA
  misc_feature 143..154: note = RNA

SEQ ID NO: 110 - DNA, 154 bp, synthetic construct; identical to SEQ ID NO: 108
  source 1..154: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked thymine
  modified_base 138: mod_base = OTHER; note = Locked nucleic acid
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139..144: note = DNA
  misc_feature 145..154: note = RNA

SEQ ID NO: 111 - DNA, 154 bp, synthetic construct; identical to SEQ ID NO: 108
  source 1..154: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked thymine
  modified_base 138: mod_base = OTHER; note = Locked nucleic acid
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139..150: note = DNA
  misc_feature 151..154: note = RNA

SEQ ID NO: 112 - DNA, 152 bp, synthetic construct
  source 1..152: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked nucleic acid
  modified_base 138: mod_base = OTHER; note = Locked nucleic acid
  modified_base 148: mod_base = OTHER; note = Nucleotide with phosphorothioate linkage
  modified_base 149: mod_base = OTHER; note = 2'-O-methyl modified nucleotide with phosphorothioate linkage
  modified_base 150: mod_base = OTHER; note = 2'-O-methyl modified nucleotide with phosphorothioate linkage
  modified_base 151: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139: note = DNA
  misc_feature 140..152: note = RNA
gctggccccccaccgccccagttttagagctagaaatagcaagttaaaataaggctagtccgttatcgacttgaaaaagtcggaccgagtcggtccgtggttccgggctgcatgaggagaccgccgtcgtcgacaagccggcggtgggtttt

SEQ ID NO: 113 - DNA, 152 bp, synthetic construct; identical to SEQ ID NO: 112
  source 1..152: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked nucleic acid
  modified_base 138: mod_base = OTHER; note = Locked nucleic acid
  modified_base 148: mod_base = OTHER; note = Nucleotide with phosphorothioate linkage
  modified_base 149: mod_base = OTHER; note = 2'-O-methyl modified nucleotide with phosphorothioate linkage
  modified_base 150: mod_base = OTHER; note = 2'-O-methyl modified nucleotide with phosphorothioate linkage
  modified_base 151: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139..142: note = DNA
  misc_feature 143..152: note = RNA

SEQ ID NO: 114 - DNA, 152 bp, synthetic construct; identical to SEQ ID NO: 112
  source 1..152: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked nucleic acid
  modified_base 138: mod_base = OTHER; note = Locked nucleic acid
  modified_base 148: mod_base = OTHER; note = Nucleotide with phosphorothioate linkage
  modified_base 149: mod_base = OTHER; note = 2'-O-methyl modified nucleotide with phosphorothioate linkage
  modified_base 150: mod_base = OTHER; note = 2'-O-methyl modified nucleotide with phosphorothioate linkage
  modified_base 151: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139..144: note = DNA
  misc_feature 145..152: note = RNA

SEQ ID NO: 115 - DNA, 152 bp, synthetic construct; identical to SEQ ID NO: 112
  source 1..152: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Locked nucleic acid
  modified_base 118: mod_base = OTHER; note = Locked nucleic acid
  modified_base 120: mod_base = OTHER; note = Locked nucleic acid
  modified_base 122: mod_base = OTHER; note = Locked nucleic acid
  modified_base 124: mod_base = OTHER; note = Locked nucleic acid
  modified_base 126: mod_base = OTHER; note = Locked nucleic acid
  modified_base 128: mod_base = OTHER; note = Locked nucleic acid
  modified_base 130: mod_base = OTHER; note = Locked thymine
  modified_base 132: mod_base = OTHER; note = Locked nucleic acid
  modified_base 134: mod_base = OTHER; note = Locked nucleic acid
  modified_base 136: mod_base = OTHER; note = Locked nucleic acid
  modified_base 138: mod_base = OTHER; note = Locked nucleic acid
  modified_base 148: mod_base = OTHER; note = Nucleotide with phosphorothioate linkage
  modified_base 149: mod_base = OTHER; note = 2'-O-methyl modified nucleotide with phosphorothioate linkage
  modified_base 150: mod_base = OTHER; note = 2'-O-methyl modified nucleotide with phosphorothioate linkage
  modified_base 151: mod_base = OTHER; note = 2'-O-methyl modified nucleotide
  misc_feature 1..116: note = RNA
  misc_feature 117: note = DNA
  misc_feature 118: note = RNA
  misc_feature 119: note = DNA
  misc_feature 120: note = RNA
  misc_feature 121: note = DNA
  misc_feature 122: note = RNA
  misc_feature 123: note = DNA
  misc_feature 124: note = RNA
  misc_feature 125: note = DNA
  misc_feature 126: note = RNA
  misc_feature 127: note = DNA
  misc_feature 128: note = RNA
  misc_feature 129: note = DNA
  misc_feature 130: note = RNA
  misc_feature 131: note = DNA
  misc_feature 132: note = RNA
  misc_feature 133: note = DNA
  misc_feature 134: note = RNA
  misc_feature 135: note = DNA
  misc_feature 136: note = RNA
  misc_feature 137: note = DNA
  misc_feature 138: note = RNA
  misc_feature 139..148: note = DNA
  misc_feature 149..152: note = RNA

SEQ ID NO: 116 - DNA, 135 bp, synthetic construct
  source 1..135: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked thymine
  modified_base 119: mod_base = OTHER; note = Locked nucleic acid
  modified_base 131: mod_base = OTHER; note = Nucleotide with phosphorothioate linkage
  modified_base 132: mod_base = OTHER; note = 2'-O-methyl modified nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120: note = DNA
  misc_feature 121..135: note = RNA
gcgactcctggaagtggccagttttagagctagaaatagcaagttaaaataaggctagtccgttatcgacttgaaaaagtcggaccgagtcggtccggcggtctccgtcgtcaggatcatccacttccaggtttt

SEQ ID NO: 117 - DNA, 135 bp, synthetic construct; identical to SEQ ID NO: 116
  source 1..135: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked thymine
  modified_base 119: mod_base = OTHER; note = Locked nucleic acid
  modified_base 131: mod_base = OTHER; note = Nucleotide with phosphorothioate linkage
  modified_base 132: mod_base = OTHER; note = 2'-O-methyl modified nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120..123: note = DNA
  misc_feature 124..135: note = RNA

SEQ ID NO: 118 - DNA, 135 bp, synthetic construct; identical to SEQ ID NO: 116
  source 1..135: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked thymine
  modified_base 119: mod_base = OTHER; note = Locked nucleic acid
  modified_base 131: mod_base = OTHER; note = Nucleotide with phosphorothioate linkage
  modified_base 132: mod_base = OTHER; note = 2'-O-methyl modified nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120..125: note = DNA
  misc_feature 126..135: note = RNA

SEQ ID NO: 119 - DNA, 135 bp, synthetic construct; identical to SEQ ID NO: 116
  source 1..135: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked thymine
  modified_base 119: mod_base = OTHER; note = Locked nucleic acid
  modified_base 131: mod_base = OTHER; note = Nucleotide with phosphorothioate linkage
  modified_base 132: mod_base = OTHER; note = 2'-O-methyl modified nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120..131: note = DNA
  misc_feature 132..135: note = RNA

SEQ ID NO: 120 - DNA, 133 bp, synthetic construct
  source 1..133: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked nucleic acid
  modified_base 119: mod_base = OTHER; note = Locked nucleic acid
  modified_base 129: mod_base = OTHER; note = Nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage
  modified_base 132: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120: note = DNA
  misc_feature 121..133: note = RNA
gctggccccccaccgccccagttttagagctagaaatagcaagttaaaataaggctagtccgttatcgacttgaaaaagtcggaccgagtcggtccggagaccgccgtcgtcgacaagccggcggtgggtttt

SEQ ID NO: 121 - DNA, 133 bp, synthetic construct; identical to SEQ ID NO: 120
  source 1..133: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked nucleic acid
  modified_base 119: mod_base = OTHER; note = Locked nucleic acid
  modified_base 129: mod_base = OTHER; note = Nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage
  modified_base 132: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120..123: note = DNA
  misc_feature 124..133: note = RNA

SEQ ID NO: 122 - DNA, 133 bp, synthetic construct; identical to SEQ ID NO: 120
  source 1..133: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked nucleic acid
  modified_base 119: mod_base = OTHER; note = Locked nucleic acid
  modified_base 129: mod_base = OTHER; note = Nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage
  modified_base 132: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120..125: note = DNA
  misc_feature 126..133: note = RNA

SEQ ID NO: 123 - DNA, 133 bp, synthetic construct; identical to SEQ ID NO: 120
  source 1..133: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked nucleic acid
  modified_base 109: mod_base = OTHER; note = Locked nucleic acid
  modified_base 111: mod_base = OTHER; note = Locked thymine
  modified_base 113: mod_base = OTHER; note = Locked nucleic acid
  modified_base 115: mod_base = OTHER; note = Locked nucleic acid
  modified_base 117: mod_base = OTHER; note = Locked nucleic acid
  modified_base 119: mod_base = OTHER; note = Locked nucleic acid
  modified_base 129: mod_base = OTHER; note = Nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage
  modified_base 132: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110: note = DNA
  misc_feature 111: note = RNA
  misc_feature 112: note = DNA
  misc_feature 113: note = RNA
  misc_feature 114: note = DNA
  misc_feature 115: note = RNA
  misc_feature 116: note = DNA
  misc_feature 117: note = RNA
  misc_feature 118: note = DNA
  misc_feature 119: note = RNA
  misc_feature 120..129: note = DNA
  misc_feature 130..133: note = RNA

SEQ ID NO: 124 - DNA, 57 bp, synthetic construct
  source 1..57: mol_type = other DNA
acactctttccctacacgacgctcttccgatctaacggatggatgtggcgcaggtag

SEQ ID NO: 125 - DNA, 57 bp, synthetic construct
  source 1..57: mol_type = other DNA
acactctttccctacacgacgctcttccgatctaagtgatggatgtggcgcaggtag

SEQ ID NO: 126 - DNA, 57 bp, synthetic construct
  source 1..57: mol_type = other DNA
acactctttccctacacgacgctcttccgatctagctgatggatgtggcgcaggtag

SEQ ID NO: 127 - DNA, 57 bp, synthetic construct
  source 1..57: mol_type = other DNA
acactctttccctacacgacgctcttccgatctataggatggatgtggcgcaggtag

SEQ ID NO: 128 - DNA, 57 bp, synthetic construct
  source 1..57: mol_type = other DNA
acactctttccctacacgacgctcttccgatctcggagatggatgtggcgcaggtag

SEQ ID NO: 129 - DNA, 57 bp, synthetic construct
  source 1..57: mol_type = other DNA
acactctttccctacacgacgctcttccgatctcttggatggatgtggcgcaggtag

SEQ ID NO: 130 - DNA, 57 bp, synthetic construct
  source 1..57: mol_type = other DNA
acactctttccctacacgacgctcttccgatctgaacgatggatgtggcgcaggtag

SEQ ID NO: 131 - DNA, 57 bp, synthetic construct
  source 1..57: mol_type = other DNA
acactctttccctacacgacgctcttccgatctgagtgatggatgtggcgcaggtag

SEQ ID NO: 132 - DNA, 57 bp, synthetic construct
  source 1..57: mol_type = other DNA
acactctttccctacacgacgctcttccgatctgcaggatggatgtggcgcaggtag

SEQ ID NO: 133 - DNA, 57 bp, synthetic construct
  source 1..57: mol_type = other DNA
acactctttccctacacgacgctcttccgatctgtaagatggatgtggcgcaggtag

SEQ ID NO: 134 - DNA, 57 bp, synthetic construct
  source 1..57: mol_type = other DNA
acactctttccctacacgacgctcttccgatcttacagatggatgtggcgcaggtag

SEQ ID NO: 135 - DNA, 57 bp, synthetic construct
  source 1..57: mol_type = other DNA
acactctttccctacacgacgctcttccgatcttctagatggatgtggcgcaggtag

SEQ ID NO: 136 - DNA, 56 bp, synthetic construct
  source 1..56: mol_type = other DNA
gactggagttcagacgtgtgctcttccgatctacagaggcgtatcatttcgcggat

SEQ ID NO: 137 - DNA, 56 bp, synthetic construct
  source 1..56: mol_type = other DNA
gactggagttcagacgtgtgctcttccgatctagaaaggcgtatcatttcgcggat

SEQ ID NO: 138 - DNA, 56 bp, synthetic construct
  source 1..56: mol_type = other DNA
gactggagttcagacgtgtgctcttccgatctctaaaggcgtatcatttcgcggat

SEQ ID NO: 139 - DNA, 56 bp, synthetic construct
  source 1..56: mol_type = other DNA
gactggagttcagacgtgtgctcttccgatctctgcaggcgtatcatttcgcggat

SEQ ID NO: 140 - DNA, 56 bp, synthetic construct
  source 1..56: mol_type = other DNA
gactggagttcagacgtgtgctcttccgatctgcggaggcgtatcatttcgcggat

SEQ ID NO: 141 - DNA, 56 bp, synthetic construct
  source 1..56: mol_type = other DNA
gactggagttcagacgtgtgctcttccgatctggataggcgtatcatttcgcggat

SEQ ID NO: 142 - DNA, 56 bp, synthetic construct
  source 1..56: mol_type = other DNA
gactggagttcagacgtgtgctcttccgatcttcaaaggcgtatcatttcgcggat

SEQ ID NO: 143 - DNA, 56 bp, synthetic construct
  source 1..56: mol_type = other DNA
gactggagttcagacgtgtgctcttccgatctttccaggcgtatcatttcgcggat

SEQ ID NO: 144 - DNA, 60 bp, synthetic construct
  source 1..60: mol_type = other DNA
acactctttccctacacgacgctcttccgatctaacggcatggatgagagaagcctggag

SEQ ID NO: 145 - DNA, 60 bp, synthetic construct
  source 1..60: mol_type = other DNA
acactctttccctacacgacgctcttccgatctaagtgcatggatgagagaagcctggag

SEQ ID NO: 146 - DNA, 60 bp, synthetic construct
  source 1..60: mol_type = other DNA
acactctttccctacacgacgctcttccgatctagctgcatggatgagagaagcctggag

SEQ ID NO: 147 - DNA, 60 bp, synthetic construct
  source 1..60: mol_type = other DNA
acactctttccctacacgacgctcttccgatctataggcatggatgagagaagcctggag

SEQ ID NO: 148 - DNA, 60 bp, synthetic construct
  source 1..60: mol_type = other DNA
acactctttccctacacgacgctcttccgatctcggagcatggatgagagaagcctggag

SEQ ID NO: 149 - DNA, 60 bp, synthetic construct
  source 1..60: mol_type = other DNA
acactctttccctacacgacgctcttccgatctcttggcatggatgagagaagcctggag

SEQ ID NO: 150 - DNA, 60 bp, synthetic construct
  source 1..60: mol_type = other DNA
acactctttccctacacgacgctcttccgatctgaacgcatggatgagagaagcctggag

SEQ ID NO: 151 - DNA, 60 bp, synthetic construct
  source 1..60: mol_type = other DNA
acactctttccctacacgacgctcttccgatctgagtgcatggatgagagaagcctggag

SEQ ID NO: 152 - DNA, 60 bp, synthetic construct
  source 1..60: mol_type = other DNA
acactctttccctacacgacgctcttccgatctgcaggcatggatgagagaagcctggag

SEQ ID NO: 153 - DNA, 60 bp, synthetic construct
  source 1..60: mol_type = other DNA
acactctttccctacacgacgctcttccgatctgtaagcatggatgagagaagcctggag

SEQ ID NO: 154 - DNA, 60 bp, synthetic construct
  source 1..60: mol_type = other DNA
acactctttccctacacgacgctcttccgatcttacagcatggatgagagaagcctggag

SEQ ID NO: 155 - DNA, 60 bp, synthetic construct
  source 1..60: mol_type = other DNA
acactctttccctacacgacgctcttccgatcttctagcatggatgagagaagcctggag

SEQ ID NO: 156 - DNA, 56 bp, synthetic construct
  source 1..56: mol_type = other DNA
gactggagttcagacgtgtgctcttccgatctacagcccagccaaacttgtcaacc

SEQ ID NO: 157 - DNA, 56 bp, synthetic construct
  source 1..56: mol_type = other DNA
gactggagttcagacgtgtgctcttccgatctagaacccagccaaacttgtcaacc

SEQ ID NO: 158 - DNA, 56 bp, synthetic construct
  source 1..56: mol_type = other DNA
gactggagttcagacgtgtgctcttccgatctctaacccagccaaacttgtcaacc

SEQ ID NO: 159 - DNA, 56 bp, synthetic construct
  source 1..56: mol_type = other DNA
gactggagttcagacgtgtgctcttccgatctctgccccagccaaacttgtcaacc

SEQ ID NO: 160 - DNA, 56 bp, synthetic construct
  source 1..56: mol_type = other DNA
gactggagttcagacgtgtgctcttccgatctgcggcccagccaaacttgtcaacc

SEQ ID NO: 161 - DNA, 56 bp, synthetic construct
  source 1..56: mol_type = other DNA
gactggagttcagacgtgtgctcttccgatctggatcccagccaaacttgtcaacc

SEQ ID NO: 162 - DNA, 56 bp, synthetic construct
  source 1..56: mol_type = other DNA
gactggagttcagacgtgtgctcttccgatcttcaacccagccaaacttgtcaacc

SEQ ID NO: 163 - DNA, 56 bp, synthetic construct
  source 1..56: mol_type = other DNA
gactggagttcagacgtgtgctcttccgatctttcccccagccaaacttgtcaacc

SEQ ID NO: 164 - DNA, 54 bp, synthetic construct
  source 1..54: mol_type = other DNA
acactctttccctacacgacgctcttccgatctaacgcgggaactgccgctggc

SEQ ID NO: 165 - DNA, 54 bp, synthetic construct
  source 1..54: mol_type = other DNA
acactctttccctacacgacgctcttccgatctaagtcgggaactgccgctggc

SEQ ID NO: 166 - DNA, 54 bp, synthetic construct
  source 1..54: mol_type = other DNA
acactctttccctacacgacgctcttccgatctagctcgggaactgccgctggc

SEQ ID NO: 167 - DNA, 54 bp, synthetic construct
  source 1..54: mol_type = other DNA
acactctttccctacacgacgctcttccgatctatagcgggaactgccgctggc

SEQ ID NO: 168 - DNA, 54 bp, synthetic construct
  source 1..54: mol_type = other DNA
acactctttccctacacgacgctcttccgatctcggacgggaactgccgctggc

SEQ ID NO: 169 - DNA, 54 bp, synthetic construct
  source 1..54: mol_type = other DNA
acactctttccctacacgacgctcttccgatctcttgcgggaactgccgctggc

SEQ ID NO: 170 - DNA, 54 bp, synthetic construct
  source 1..54: mol_type = other DNA
acactctttccctacacgacgctcttccgatctgaaccgggaactgccgctggc

SEQ ID NO: 171 - DNA, 54 bp, synthetic construct
  source 1..54: mol_type = other DNA
acactctttccctacacgacgctcttccgatctgagtcgggaactgccgctggc

SEQ ID NO: 172 - DNA, 54 bp, synthetic construct
  source 1..54: mol_type = other DNA
acactctttccctacacgacgctcttccgatctgcagcgggaactgccgctggc

SEQ ID NO: 173 - DNA, 54 bp, synthetic construct
  source 1..54: mol_type = other DNA
acactctttccctacacgacgctcttccgatctgtaacgggaactgccgctggc

SEQ ID NO: 174 - DNA, 54 bp, synthetic construct
  source 1..54: mol_type = other DNA
acactctttccctacacgacgctcttccgatcttacacgggaactgccgctggc

SEQ ID NO: 175 - DNA, 54 bp, synthetic construct
  source 1..54: mol_type = other DNA
acactctttccctacacgacgctcttccgatcttctacgggaactgccgctggc

SEQ ID NO: 176 - DNA, 56 bp, synthetic construct
  source 1..56: mol_type = other DNA
gactggagttcagacgtgtgctcttccgatctacaggaggaggccctcatctggcg

SEQ ID NO: 177 - DNA, 56 bp, synthetic construct
  source 1..56: mol_type = other DNA
gactggagttcagacgtgtgctcttccgatctagaagaggaggccctcatctggcg

SEQ ID NO: 178 - DNA, 56 bp, synthetic construct
  source 1..56: mol_type = other DNA
gactggagttcagacgtgtgctcttccgatctctaagaggaggccctcatctggcg

SEQ ID NO: 179 - DNA, 56 bp, synthetic construct
  source 1..56: mol_type = other DNA
gactggagttcagacgtgtgctcttccgatctctgcgaggaggccctcatctggcg

SEQ ID NO: 180 - DNA, 56 bp, synthetic construct
  source 1..56: mol_type = other DNA
gactggagttcagacgtgtgctcttccgatctgcgggaggaggccctcatctggcg

SEQ ID NO: 181 - DNA, 56 bp, synthetic construct
  source 1..56: mol_type = other DNA
gactggagttcagacgtgtgctcttccgatctggatgaggaggccctcatctggcg

SEQ ID NO: 182 - DNA, 56 bp, synthetic construct
  source 1..56: mol_type = other DNA
gactggagttcagacgtgtgctcttccgatcttcaagaggaggccctcatctggcg

SEQ ID NO: 183 - DNA, 56 bp, synthetic construct
  source 1..56: mol_type = other DNA
gactggagttcagacgtgtgctcttccgatctttccgaggaggccctcatctggcg

SEQ ID NO: 184 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked nucleic acid
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
gaagctcggaaaagcgatcgtgatgctgcagaaggacgccacaataccgcagct

SEQ ID NO: 185 - DNA, 54 bp, synthetic construct; identical to SEQ ID NO: 184
  source 1..54: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 52: mod_base = OTHER; note = Nucleotide with phosphorothioate linkage
  modified_base 53: mod_base = OTHER; note = Nucleotide with phosphorothioate linkage

SEQ ID NO: 186 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
ggcggtctccgtcgtcaggatcatccacttccaggacgccacaataccgcagct

SEQ ID NO: 187 - DNA, 52 bp, synthetic construct
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked nucleic acid
ggagaccgccgtcgtcgacaagccggcggtgggtcatgcagcccggaaccac

SEQ ID NO: 188 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
ggagaccgccgtcgtcgacaagcccgtgctcagtcacgccacaataccgcagct

SEQ ID NO: 189 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
ggcggtctccgtcgtcaggatcatccgggatactgtcatgcagcccggaaccac

SEQ ID NO: 190 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
ggcggtctccgtcgtcaggatcatccacttccaggtcatgcagcccggaaccac

SEQ ID NO: 191 - DNA, 52 bp, synthetic construct
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked thymine
ggagaccgccgtcgtcgacaagccggcggtgggacgccacaataccgcagct

SEQ ID NO: 192 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
ggagaccgccgtcgtcgacaagcccgtgctcagtctcatgcagcccggaaccac

SEQ ID NO: 193 - DNA, 54 bp, synthetic construct
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
ggcggtctccgtcgtcaggatcatccgggatactgacgccacaataccgcagct

SEQ ID NO: 194 - DNA, 54 bp, synthetic construct; identical to SEQ ID NO: 186
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..29: note = DNA
  misc_feature 30..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 195 - DNA, 52 bp, synthetic construct; identical to SEQ ID NO: 187
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..29: note = DNA
  misc_feature 30..33: note = RNA
  misc_feature 34..39: note = DNA
  misc_feature 40: note = RNA
  misc_feature 41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA

SEQ ID NO: 196 - DNA, 54 bp, synthetic construct; identical to SEQ ID NO: 188
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..29: note = DNA
  misc_feature 30..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 197 - DNA, 54 bp, synthetic construct; identical to SEQ ID NO: 189
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..29: note = DNA
  misc_feature 30..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 198 - DNA, 54 bp, synthetic construct; identical to SEQ ID NO: 186
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine

SEQ ID NO: 199 - DNA, 52 bp, synthetic construct; identical to SEQ ID NO: 187
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked nucleic acid

SEQ ID NO: 200 - DNA, 54 bp, synthetic construct; identical to SEQ ID NO: 188
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine

SEQ ID NO: 201 - DNA, 54 bp, synthetic construct; identical to SEQ ID NO: 189
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid

SEQ ID NO: 202 - DNA, 54 bp, synthetic construct; identical to SEQ ID NO: 186
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 25: mod_base = OTHER; note = Locked nucleic acid
  modified_base 27: mod_base = OTHER; note = Locked nucleic acid
  modified_base 29: mod_base = OTHER; note = Locked thymine
  modified_base 31: mod_base = OTHER; note = Locked nucleic acid
  modified_base 33: mod_base = OTHER; note = Locked nucleic acid
  modified_base 35: mod_base = OTHER; note = Locked nucleic acid
  modified_base 52: mod_base = OTHER; note = Nucleotide with phosphorothioate linkage
  modified_base 53: mod_base = OTHER; note = Nucleotide with phosphorothioate linkage

SEQ ID NO: 203 - DNA, 52 bp, synthetic construct; identical to SEQ ID NO: 187
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 25: mod_base = OTHER; note = Locked nucleic acid
  modified_base 27: mod_base = OTHER; note = Locked nucleic acid
  modified_base 29: mod_base = OTHER; note = Locked nucleic acid
  modified_base 31: mod_base = OTHER; note = Locked nucleic acid
  modified_base 33: mod_base = OTHER; note = Locked nucleic acid
  modified_base 50: mod_base = OTHER; note = Nucleotide with phosphorothioate linkage
  modified_base 51: mod_base = OTHER; note = Nucleotide with phosphorothioate linkage

SEQ ID NO: 204 - DNA, 54 bp, synthetic construct; identical to SEQ ID NO: 188
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 25: mod_base = OTHER; note = Locked nucleic acid
  modified_base 27: mod_base = OTHER; note = Locked thymine
  modified_base 29: mod_base = OTHER; note = Locked nucleic acid
  modified_base 31: mod_base = OTHER; note = Locked nucleic acid
  modified_base 33: mod_base = OTHER; note = Locked nucleic acid
  modified_base 35: mod_base = OTHER; note = Locked nucleic acid
  modified_base 52: mod_base = OTHER; note = Nucleotide with phosphorothioate linkage
  modified_base 53: mod_base = OTHER; note = Nucleotide with phosphorothioate linkage

SEQ ID NO: 205 - DNA, 54 bp, synthetic construct; identical to SEQ ID NO: 189
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 25: mod_base = OTHER; note = Locked nucleic acid
  modified_base 27: mod_base = OTHER; note = Locked nucleic acid
  modified_base 29: mod_base = OTHER; note = Locked nucleic acid
  modified_base 31: mod_base = OTHER; note = Locked thymine
  modified_base 33: mod_base = OTHER; note = Locked nucleic acid
  modified_base 35: mod_base = OTHER; note = Locked nucleic acid
  modified_base 52: mod_base = OTHER; note = Nucleotide with phosphorothioate linkage
  modified_base 53: mod_base = OTHER; note = Nucleotide with phosphorothioate linkage

SEQ ID NO: 206 - DNA, 54 bp, synthetic construct; identical to SEQ ID NO: 186
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24: note = DNA
  misc_feature 25..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 207 - DNA, 54 bp, synthetic construct; identical to SEQ ID NO: 186
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..27: note = DNA
  misc_feature 28..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 208 - DNA, 54 bp, synthetic construct; identical to SEQ ID NO: 186
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..31: note = DNA
  misc_feature 32..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 209 - DNA, 52 bp, synthetic construct; identical to SEQ ID NO: 187
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24: note = DNA
  misc_feature 25..33: note = RNA
  misc_feature 34..39: note = DNA
  misc_feature 40: note = RNA
  misc_feature 41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA

SEQ ID NO: 210 - DNA, 52 bp, synthetic construct; identical to SEQ ID NO: 187
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..27: note = DNA
  misc_feature 28..33: note = RNA
  misc_feature 34..39: note = DNA
  misc_feature 40: note = RNA
  misc_feature 41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA

SEQ ID NO: 211 - DNA, 52 bp, synthetic construct; identical to SEQ ID NO: 187
  source 1..52: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 40: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid with phosphorothioate linkage
  modified_base 51: mod_base = OTHER; note = Nucleotide with phosphorothioate linkage
  modified_base 52: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..31: note = DNA
  misc_feature 32..33: note = RNA
  misc_feature 34..39: note = DNA
  misc_feature 40: note = RNA
  misc_feature 41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA

SEQ ID NO: 212 - DNA, 54 bp, synthetic construct; identical to SEQ ID NO: 188
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24: note = DNA
  misc_feature 25..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 213 - DNA, 54 bp, synthetic construct; identical to SEQ ID NO: 188
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..27: note = DNA
  misc_feature 28..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 214 - DNA, 54 bp, synthetic construct; identical to SEQ ID NO: 188
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked thymine
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..31: note = DNA
  misc_feature 32..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 215 - DNA, 54 bp, synthetic construct; identical to SEQ ID NO: 189
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24: note = DNA
  misc_feature 25..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 216 - DNA, 54 bp, synthetic construct; identical to SEQ ID NO: 189
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..27: note = DNA
  misc_feature 28..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 217 - DNA, 54 bp, synthetic construct; identical to SEQ ID NO: 189
  source 1..54: mol_type = other DNA
  modified_base 1: mod_base = OTHER; note = Locked nucleic acid with phosphorothioate linkage
  modified_base 2: mod_base = OTHER; note = Nucleotide with phosphorothioate linkage
  modified_base 3: mod_base = OTHER; note = Locked nucleic acid
  modified_base 5: mod_base = OTHER; note = Locked nucleic acid
  modified_base 7: mod_base = OTHER; note = Locked nucleic acid
  modified_base 9: mod_base = OTHER; note = Locked nucleic acid
  modified_base 11: mod_base = OTHER; note = Locked nucleic acid
  modified_base 13: mod_base = OTHER; note = Locked nucleic acid
  modified_base 15: mod_base = OTHER; note = Locked thymine
  modified_base 17: mod_base = OTHER; note = Locked nucleic acid
  modified_base 19: mod_base = OTHER; note = Locked nucleic acid
  modified_base 21: mod_base = OTHER; note = Locked thymine
  modified_base 23: mod_base = OTHER; note = Locked nucleic acid
  modified_base 42: mod_base = OTHER; note = Locked nucleic acid
  modified_base 44: mod_base = OTHER; note = Locked nucleic acid
  modified_base 46: mod_base = OTHER; note = Locked nucleic acid
  modified_base 48: mod_base = OTHER; note = Locked nucleic acid
  modified_base 50: mod_base = OTHER; note = Locked nucleic acid
  modified_base 52: mod_base = OTHER; note = Locked nucleic acid with phosphorothioate linkage
  modified_base 53: mod_base = OTHER; note = Nucleotide with phosphorothioate linkage
  modified_base 54: mod_base = OTHER; note = Locked nucleic acid
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15: note = RNA
  misc_feature 16: note = DNA
  misc_feature 17: note = RNA
  misc_feature 18: note = DNA
  misc_feature 19: note = RNA
  misc_feature 20: note = DNA
  misc_feature 21: note = RNA
  misc_feature 22: note = DNA
  misc_feature 23: note = RNA
  misc_feature 24..31: note = DNA
  misc_feature 32..35: note = RNA
  misc_feature 36..41: note = DNA
  misc_feature 42: note = RNA
  misc_feature 43: note = DNA
  misc_feature 44: note = RNA
  misc_feature 45: note = DNA
  misc_feature 46: note = RNA
  misc_feature 47: note = DNA
  misc_feature 48: note = RNA
  misc_feature 49: note = DNA
  misc_feature 50: note = RNA
  misc_feature 51: note = DNA
  misc_feature 52: note = RNA
  misc_feature 53: note = DNA
  misc_feature 54: note = RNA

SEQ ID NO: 218 - DNA, 24 bp, synthetic construct
  source 1..24: mol_type = other DNA
  modified_base 1: mod_base = OTHER; note = 5' phosphate modified nucleotide
atcacgatcgcttttccgagcttc

SEQ ID NO: 219 - DNA, 24 bp, synthetic construct
  source 1..24: mol_type = other DNA
  modified_base 1: mod_base = OTHER; note = 5' phosphate modified nucleotide with phosphorothioate linkage
  modified_base 2: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
atcacgatcgcttttccgagcttc

SEQ ID NO: 220 - DNA, 24 bp, synthetic construct
  source 1..24: mol_type = other DNA
  modified_base 1: mod_base = OTHER; note = 5' phosphate modified nucleotide
  modified_base 22: mod_base = OTHER; note = Nucleotide with phosphorothioate linkage
  modified_base 23: mod_base = OTHER; note = Nucleotide with phosphorothioate linkage
atcacgatcgcttttccgagcttc

SEQ ID NO: 221 - DNA, 24 bp, synthetic construct
  source 1..24: mol_type = other DNA
  modified_base 1: mod_base = OTHER; note = 5' phosphate modified nucleotide
atgatcctgacgacggagaccgcc

SEQ ID NO: 222 - DNA, 24 bp, synthetic construct
  source 1..24: mol_type = other DNA
  modified_base 1: mod_base = OTHER; note = 5' phosphate modified nucleotide
ggcttgtcgacgacggcggtctcc

SEQ ID NO: 223 - DNA, 24 bp, synthetic construct
  source 1..24: mol_type = other DNA
  modified_base 1: mod_base = OTHER; note = 5' phosphate modified nucleotide
  modified_base 22: mod_base = OTHER; note = Nucleotide with phosphorothioate linkage
  modified_base 23: mod_base = OTHER; note = Nucleotide with phosphorothioate linkage
atgatcctgacgacggagaccgcc

SEQ ID NO: 224 - DNA, 24 bp, synthetic construct
  source 1..24: mol_type = other DNA
  modified_base 1: mod_base = OTHER; note = 5' phosphate modified nucleotide
  modified_base 22: mod_base = OTHER; note = Nucleotide with phosphorothioate linkage
  modified_base 23: mod_base = OTHER; note = Nucleotide with phosphorothioate linkage
ggcttgtcgacgacggcggtctcc

SEQ ID NO: 225 - DNA, 24 bp, synthetic construct
  source 1..24: mol_type = other DNA
  modified_base 1: mod_base = OTHER; note = 5' phosphate modified nucleotide
  modified_base 6: mod_base = OTHER; note = 5-methyl modified nucleotide
  modified_base 7: mod_base = OTHER; note = 5-methyl modified nucleotide
  modified_base 11: mod_base = OTHER; note = 5-methyl modified nucleotide
  modified_base 14: mod_base = OTHER; note = 5-methyl modified nucleotide
  modified_base 20: mod_base = OTHER; note = 5-methyl modified nucleotide
  modified_base 21: mod_base = OTHER; note = 5-methyl modified nucleotide
  modified_base 23: mod_base = OTHER; note = 5-methyl modified nucleotide
  modified_base 24: mod_base = OTHER; note = 3' 5-methyl modified nucleotide
atgatcctgacgacggagaccgcc

SEQ ID NO: 226 - DNA, 24 bp, synthetic construct
  source 1..24: mol_type = other DNA
  modified_base 1: mod_base = OTHER; note = 5' phosphate modified nucleotide
  modified_base 3: mod_base = OTHER; note = 5-methyl modified nucleotide
  modified_base 8: mod_base = OTHER; note = 5-methyl modified nucleotide
  modified_base 11: mod_base = OTHER; note = 5-methyl modified nucleotide
  modified_base 14: mod_base = OTHER; note = 5-methyl modified nucleotide
  modified_base 17: mod_base = OTHER; note = 5-methyl modified nucleotide
  modified_base 21: mod_base = OTHER; note = 5-methyl modified nucleotide
  modified_base 23: mod_base = OTHER; note = 5-methyl modified nucleotide
  modified_base 24: mod_base = OTHER; note = 3' 5-methyl modified nucleotide
ggcttgtcgacgacggcggtctcc

SEQ ID NO: 227 - DNA, 24 bp, synthetic construct
  source 1..24: mol_type = other DNA
  modified_base 1: mod_base = OTHER; note = 5' phosphate modified nucleotide
  modified_base 6: mod_base = OTHER; note = 5-methyl modified nucleotide
  modified_base 7: mod_base = OTHER; note = 5-methyl modified nucleotide
  modified_base 11: mod_base = OTHER; note = 5-methyl modified nucleotide
  modified_base 14: mod_base = OTHER; note = 5-methyl modified nucleotide
  modified_base 20: mod_base = OTHER; note = 5-methyl modified nucleotide
  modified_base 21: mod_base = OTHER; note = 5-methyl modified nucleotide
  modified_base 22: mod_base = OTHER; note = Nucleotide with phosphorothioate linkage
  modified_base 23: mod_base = OTHER; note = 5-methyl modified nucleotide with phosphorothioate linkage
  modified_base 24: mod_base = OTHER; note = 3' 5-methyl modified nucleotide
atgatcctgacgacggagaccgcc

SEQ ID NO: 228 - DNA, 24 bp, synthetic construct
  source 1..24: mol_type = other DNA
  modified_base 1: mod_base = OTHER; note = 5' phosphate modified nucleotide
  modified_base 3: mod_base = OTHER; note = 5-methyl modified nucleotide
  modified_base 8: mod_base = OTHER; note = 5-methyl modified nucleotide
  modified_base 11: mod_base = OTHER; note = 5-methyl modified nucleotide
  modified_base 14: mod_base = OTHER; note = 5-methyl modified nucleotide
  modified_base 17: mod_base = OTHER; note = 5-methyl modified nucleotide
  modified_base 21: mod_base = OTHER; note = 5-methyl modified nucleotide
  modified_base 22: mod_base = OTHER; note = Nucleotide with phosphorothioate linkage
  modified_base 23: mod_base = OTHER; note = 5-methyl modified nucleotide with phosphorothioate linkage
  modified_base 24: mod_base = OTHER; note = 3' 5-methyl modified nucleotide
ggcttgtcgacgacggcggtctcc

SEQ ID NO: 229 - protein, 10 aa, synthetic construct
  VARIANT 1..10: note = This sequence may encompass 1-5 GS repeating units
GSGSGSGSGS

SEQ ID NO: 230 - protein, 15 aa, synthetic construct
  VARIANT 1..15: note = This sequence may encompass 1-5 GGS repeating units
GGSGGSGGSGGSGGS

(19) United States
(12) Patent Application Publication
(10) Pub. No.: US 2026/0193631 A1
(43) Pub. Date: Jul. 9, 2026

(54) DNA LIGASE COMPOSITIONS, METHODS, AND USES THEREOF

(71) Applicant: Revision Bio Corporation, Cambridge, MA (US)

(72) Inventors: Shantanu KUMAR, Hipkinton, MA (US); JOnathan Hsu, Cambridge, MA (US)

(21) Appl. No.: 19/572,056

(22) Filed: Mar. 19, 2026

Related U.S. Application Data
(63) Continuation of application No. PCT/US24/47704, filed on Sep. 20, 2024.
(60) Provisional application No. 63/584,712, filed on Sep. 22, 2023.

(51) Int. Cl. C12N 9/22; C12N 9/00; C12N 15/113; C12N 15/52; C12N 15/62

(52) U.S. Cl. CPC C12N 9/226; C12N 9/93; C12N 15/113; C12N 15/52; C12N 15/62; C12Y 605/01001; C12N 2310/20; C12N 2310/531; C12N 2840/44

(57) ABSTRACT

DNA ligase compositions, guide polynucleotides, donor nucleic acids, systems, methods, and uses thereof are provided. Methods of modifying a target nucleic acid and genetically modifying a cell are described. The methods can be used for biotechnology applications, therapeutic treatments and for generating cell therapies for the treatment of various diseases and conditions. Also included are scaffolds and kits comprising the compositions and systems described herein.

### CROSS-REFERENCE

This application claims the benefit claims of and is a continuation of International Application No. PCT/US2024/047704, filed Sep. 20, 2024, which claims the benefit of and priority to U.S. Provisional Application No. 63/584,712 filed Sep. 22, 2023, the contents of each of which is incorporated herein by reference in its entirety.

### SEQUENCE LISTING

The instant application contains a Sequence Listing which has been submitted electronically in xml format and is hereby incorporated by reference in its entirety. Said xml copy, created on Sep. 19, 2024, is named 219001_702601_SL.xml and is 1,265,664 bytes in size.

### BACKGROUND

While many CRISPR systems have emerged as a useful tool for gene editing, such systems are limited by off-target effects, the length of insertion sequences, and inefficient nucleic acid editing with low fidelity. Therefore, there is a great unmet need for gene modification systems that can address these concerns.

### SUMMARY

Provided herein are compositions, wherein the compositions comprise: (a) a donor nucleic acid; (b) a polynucleotide encoding a protein construct comprising a nickase or a variant thereof and a DNA ligase or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: a deoxyribonucleotide and a ribonucleotide, wherein the ligation splint 1 region has complementarity to the target nucleic acid.

Provided herein are engineered fusion proteins, wherein the engineered fusion proteins comprise: (a) a DNA ligase or a functional fragment thereof; and (b) an engineered nickase that comprises three amino acid substitutions at positions corresponding to amino acid positions 221, 394, and 840 of a nuclease comprising a sequence of SEQ ID NO: 69.

Provided herein are systems for modifying a target nucleic acid, the systems comprising: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region has complementarity to the target nucleic acid, wherein upon introduction to a cell or a cell-free system, the system incorporates the donor nucleic acid into the target nucleic acid, thereby modifying the target nucleic acid.

Provided herein are systems for modifying a target nucleic acid, the systems comprising: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: one or more ribonucleotides or one or more deoxyribonucleotides, wherein the ligation splint 1 region has complementarity to the target nucleic acid, wherein upon introduction to a cell or a cell-free system, the system incorporates the donor nucleic acid into the target nucleic acid, thereby modifying the target nucleic acid.

Provided herein are systems for modifying a target nucleic acid, the systems comprising: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: one or more deoxyribonucleotides, wherein the ligation splint 1 region has complementarity to the target nucleic acid, wherein upon introduction to a cell or a cell-free system, the system incorporates the donor nucleic acid into the target nucleic acid, thereby modifying the target nucleic acid.

Provided herein are systems for modifying a target nucleic acid, wherein the systems comprise: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: a deoxyribonucleotide and a ribonucleotide, wherein the ligation splint 1 region has complementarity to the target nucleic acid, wherein upon introduction to a cell or a cell-free system, the system incorporates the donor nucleic acid into the target nucleic acid, thereby modifying the target nucleic acid.

Provided herein are compositions comprising: wherein the compositions comprise: a system provided herein, an engineered protein construct provided herein, a DNA ligase provided herein or a functional fragment thereof, or a guide polynucleotide provided herein.

Provided herein are compositions, wherein the compositions comprise: the systems provided herein; and a delivery vehicle.

Provided herein are polynucleotides, wherein the polynucleotides encode for a system provided herein, an engineered protein provided herein, or a guide polynucleotide provided herein.

Provided herein are sets of polynucleotides, wherein the sets of polynucleotides encode for a system provided herein, an engineered protein provided herein, or a guide polynucleotide provided herein.

Provided herein are nanoparticles, wherein the nanoparticles comprise: the polynucleotides provided herein, the sets of polynucleotides provided herein, the systems provided herein, the compositions provided herein, the cells provided herein, the vectors provided herein or any portion thereof.

Provided herein are vectors, wherein the vectors comprise a polynucleotide provided herein or the set of polynucleotides provided herein.

Provided herein are cells, wherein the cells comprise: a system provided herein, an engineered proteins provided herein, a composition provided herein, a vector provided herein, or a guide polynucleotide provided herein.

Provided herein are compositions, wherein the compositions comprise: a donor nucleic acid, and a guide polynucleotide or a polynucleotide encoding the guide polynucleotide, wherein the guide polynucleotide comprises in 5′ to 3′ order: (a) a targeting region that has complementarity to a target nucleic acid; (b) a protein binding region, wherein the protein binding region comprises a secondary structure that binds to a protein; (c) a ligation splint 2 region, wherein the ligation splint 2 region has complementarity to the donor nucleic acid and has complementarity to the target nucleic acid and at least one alteration relative to the target nucleic acid or at least one nucleic acid strand thereof; and (d) a ligation splint 1 region, wherein the ligation splint 1 region comprises one or more deoxynucleotides or one or more ribonucleotides.

Provided herein are compositions, wherein the compositions comprise: a donor nucleic acid, and a guide polynucleotide or a polynucleotide encoding the guide polynucleotide, wherein the guide polynucleotide comprises in 5′ to 3′ order: (a) a targeting region that has complementarity to a target nucleic acid; (b) a protein binding region, wherein the protein binding region comprises a secondary structure that binds to a protein; (c) a ligation splint 2 region, wherein the ligation splint 2 region has complementarity to the donor nucleic acid and has complementarity to the target nucleic acid and at least one alteration relative to the target nucleic acid or at least one nucleic acid strand thereof; and (d) a ligation splint 1 region, wherein the ligation splint 1 region comprises a deoxyribonucleotide and a ribonucleotide.

Provided herein are methods of ligating a donor nucleic acid with a target nucleic acid, wherein the methods comprise: contacting a cell or a cell-free system with: (a) a donor nucleic acid, (b) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a ligation splint 2 region, wherein the ligation splint 2 region is complementary to the donor nucleic acid and has complementarity to the target nucleic acid and at least one alteration relative to the target nucleic acid; (iv) a ligation splint 1 region, wherein the ligation splint 1 region comprises one or more deoxyribonucleotides or one or more ribonucleotides. (c) an engineered protein or a polynucleotide encoding the engineered protein, wherein the engineered protein comprises: (i) a nickase region; and (ii) a DNA ligase region; wherein: the guide polynucleotide forms a complex with the engineered protein via the protein binding region, the targeting sequence forms a complex with the complementary strand, the nickase region of the engineered protein generates a break in the target nucleic acid to generate a leading strand and a complementary strand, the ligation splint 1 region forms a complex with the leading strand, and wherein the DNA ligase region attaches the donor nucleic acid to the leading strand, thereby ligating the donor nucleic acid to the target nucleic acid.

Provided herein are methods, wherein the methods comprise: administering to a subject, an organ, a tissue, or a cell the system provided herein, the composition provided herein, or the cell provided herein, wherein the administering modifies a gene in the subject, the organ, the tissue, or the cell.

Provided herein are methods, wherein the methods comprise: contacting a cell or a population of cells with the system provided herein or the composition provided herein, thereby modifying a gene in the cell.

Provided herein are populations of cells, wherein the populations of cells are made by the methods provided herein.

Provided herein are kits, wherein the kits comprise: a first container comprising: a donor nucleic acid, and a guide polynucleotide or a polynucleotide encoding the guide polynucleotide, wherein the guide polynucleotide comprises: (i) a targeting region that has complementarity to the target nucleic acid; (ii) a protein binding region, wherein the protein binding region comprises a secondary structure that binds to a nuclease or a nickase; (iii) a ligation splint 2 region, wherein the donor hybridizing region has complementarity to the donor nucleic acid and has complementarity to a target nucleic acid, and wherein the ligation splint 2 region comprises at least one mismatch nucleobase relative to the target nucleic acid; and (iv) a ligation splint 1 region, wherein the ligation splint 1 region comprises deoxyribonucleotides and ribonucleotides, and a second container comprising: an engineered polypeptide comprising a nickase operably linked to a DNA ligase.

Provided herein are scaffolds, wherein the scaffolds comprise: the system provided herein or the composition provided herein; and a solid surface, wherein the system or the composition are immobilized to the solid surface.

### A BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B show schematics of an embodiment of the system provided herein. FIG. 1A shows a schematic of the DNA editing process by an engineered protein comprising a Cas9-nickase-DNA ligase and a guide polynucleotide designed to form a complex with a double stranded target nucleic acid and edit the target nucleic acid by ligating a donor nucleic acid to the target nucleic acid sequence. FIG. 1B shows a schematic of the ligation editing guide polynucleotide (legRNA) for DNA editing by DNA ligase. The legRNA comprises a ligation splint 2 region (LS2) and a ligation splint 1 region (LS1). Key: D is a deoxyribonucleotide (DNA) and R is a ribonucleotide (RNA).

FIG. 2 is an image of a denaturing urea polyacrylamide gel showing the targeted DNA editing by DNA ligase with splint oligonucleotide in trans. Lanes 1 and 12 show the oligo ladder of non-nicked (Oligo 1, 54 nucleotides (nt)) and pre-nicked (Oligo 32, 34 nt) DNA. The bands in lane 2 represent the Cas9 nickase (H840A) control using in vitro transcribed single guide RNA. The bands in lane 3 represent the positive control with insertion of 15 nt DNA writing (49 nt) (Oligo 32, Oligo 11, Oligo 22) and spiked-in oligo ladder for non-nicked (Oligo 1, 54 nt) and pre-nicked (Oligo 32, 34 nt) DNA. The bands in lane 4 represent the positive control with insertion of 90 nt DNA writing (49 nt) (Oligo 32, Oligo 11, Oligo 22) and spiked-in oligo ladder for non-nicked (Oligo 1, 54 nt) and pre-nicked (Oligo 32, 34 nt) DNA. The bands in lane 5 represent the negative control DNA ligase editing without Cas9 nickase (H840A). The bands in lanes 6-11 represent the products of DNA ligation editing with DNA ligation splint in trans with Oligo 22 and ligation donor oligonucleotide with Oligo 11 (lane 6), Oligo 12 (lane 7), Oligo 13 (lane 8), Oligo 14 (lane 9), Oligo 15 (lane 10), or Oligo 16 (lane 11).

FIG. 3 is an image of a denaturing urea polyacrylamide gel showing the targeted DNA editing by DNA ligase with a synthetic single guide polynucleotide. The bands in lanes 1 and 12 represent an oligo ladder showing non-nicked (Oligo 1, 54 nt) and pre-nicked (Oligo 2, 34 nt) DNA. The bands in lane 2 represent Cas9 nickase (H840A) control using Guide 3. The bands in lanes 3-11 represent the products of DNA ligation editing with Guide 1 (lane 3), Guide 2 (lane 4), Guide 3 (lane 5), Guide 4 (lane 6), Guide 5 (lane 7), Guide 6 (lane 8), Guide 7 (lane 9), Guide 8 (lane 10), or Guide 9 (lane 11).

FIG. 4 is a bar plot showing DNA ligase genome editing efficiency in human cells with three engineered protein constructs. Y-axis represents desired editing efficiency (%). X-axis represents engineered protein constructs (Constructs 10-12; SEQ ID NOS: 98-100).

FIG. 5 is a bar plot showing DNA ligase genome editing efficiency in human cells at FANCF site 1 (gRVB_3) using the Chlorella DNA ligase editor construct (Construct 10) and various combinations of splint and DNA donor modifications. Y-axis represents desired editing efficiency (%). X-axis represents combinations of splint and DNA donor modifications, including oRVB_61/oRVB_95 (SEQ ID NO: 184/SEQ ID NO: 218), oRVB_61/oRVB_96 (SEQ ID NO: 184/SEQ ID NO: 219), oRVB_61/oRVB_97 (SEQ ID NO: 184/SEQ ID NO: 220), oRVB_62/oRVB_95 (SEQ ID NO: 185/SEQ ID NO: 218), oRVB_62/oRVB_96 (SEQ ID NO: 185/SEQ ID NO: 219), and oRVB_62/oRVB_97 (SEQ ID NO: 185/SEQ ID NO: 220).

FIG. 6 is a bar plot showing DNA ligase genome editing efficiency in human cells at HEK site 3 in human cells using the Chlorella DNA ligase editor construct (Construct 10) and various splint constructs with and without RNA. Y-axis represents desired editing efficiency (%). X-axis represents various splint constructs with and without RNA, including oRVB_81/oRVB_82 (SEQ ID NO: 204/SEQ ID NO: 205), oRVB_69/oRVB_70 (SEQ ID NO: 192/SEQ ID NO: 193), oRVB_77/oRVB_78 (SEQ ID NO: 200/SEQ ID NO: 201), oRVB_65/oRVB_66 (SEQ ID NO: 188/SEQ ID NO: 189), and oRVB_73/oRVB_74 (SEQ ID NO: 196/SEQ ID NO: 197).

FIG. 7 is a bar plot showing DNA ligase genome editing efficiency in human cells at AAV site 1 in human cells using the Chlorella DNA ligase editor construct (Construct 10) and various splint constructs with and without RNA. Y-axis represents desired editing efficiency (%). X-axis represents splint constructs with and without RNA, including oRVB_79/oRVB_80 (SEQ ID NO: 202/SEQ ID NO: 203), oRVB_75/oRVB_76 (SEQ ID NO: 198/SEQ ID NO: 199), oRVB_63/oRVB_64 (SEQ ID NO: 186/SEQ ID NO: 187), oRVB_67/oRVB_68 (SEQ ID NO: 190/SEQ ID NO: 191), and oRVB_71/oRVB_72 (SEQ ID NO: 194/SEQ ID NO: 195).

FIG. 8 is a bar plot showing DNA ligase genome editing efficiency in human cells at AAV site 1 using the Chlorella DNA ligase editor construct (Construct 10) and guide RNAs (legRNAs) with splint region incorporated with various degrees of RNA. Y-axis represents desired editing efficiency (%). X-axis represents guide RNA constructs, including gRVB_9/gRVB_13 (SEQ ID NO: 110/SEQ ID NO: 114), gRVB_16/gRVB_20 (SEQ ID NO: 117/SEQ ID NO: 121), and gRVB_17/gRVB_21 (SEQ ID NO: 118/SEQ ID NO: 122).

Various aspects now will be described more fully hereinafter. Such aspects may, however, be embodied in many different forms and should not be construed as limited to the embodiments set forth herein.

### DETAILED DESCRIPTION OF THE INVENTION

Provided herein are compositions, kits, methods, and uses thereof for editing and ligating a donor nucleic acid sequence to a target gene using a DNA ligase. Briefly, further described herein are: (1) gene editing systems; (2) delivery vehicles and vectors; (3) cells and cell-free systems; (4) pharmaceutical compositions, dosing, and administration; (5) scaffolds and systems; (6) kits; (7) gene editing activity; and (8) applications.

Provided herein are compositions and systems for gene editing that can be used in a variety of applications, such as gene editing, diagnostics, and biologics manufacturing. The compositions and systems provided herein include an engineered protein that comprises nickase activity and DNA ligase activity that allows the engineered protein to bind to dsDNA and create a nick on the non-target DNA strand to modify the target nucleic acid.

The compositions and systems provided herein also include a guide polynucleotide and a donor nucleic acid for incorporation into the target nucleic acid. The guide polynucleotide binds to the engineered protein and the target nucleic acid to promote loop formation between the cleaved target nucleic acid and the guide polynucleotide to promote high-fidelity gene editing. The guide polynucleotide mediates hybridization of the donor nucleic acid, bringing the 3′ end of the cleavage site of the target nucleic acid and the 5′ end of the donor nucleic acid in proximity to each other. The target nucleic acid and guide polynucleotide form a complex to provide a substrate for DNA ligase to directly attach the donor nucleic acid sequence of the guide nucleic acid and the 3′ end of the cleavage site. The compositions and systems provided herein allow for controlled editing outcomes and have limited off-target effects. Furthermore, the compositions and systems provided herein allow for the insertion of large nucleic acid sequences that can be integrated into the target nucleic acid with precision.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each were incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to +20% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve or at least partially achieve the desired effect.

As used herein, the term “leading strand” and its grammatical equivalents refers to a single-stranded nucleic acid sequence that binds to a ligation splint 1 region (also referred to herein as a splint) or a ligation splint 2 region of a guide polynucleotide provided herein.

As used herein, the term “complementary strand” and its grammatical equivalents refers to a single-stranded nucleic acid sequence that comprises a protospacer adjacent motif (PAM), is complementary to the leading strand, and/or binds to a targeting region of a guide polynucleotide provided herein.

The term “effective amount” or “therapeutically effective amount” refers to an amount that is sufficient to achieve or at least partially achieve the desired effect.

### (1) Gene Editing Systems

Provided herein are systems, wherein the systems comprise: a donor nucleic acid, a guide polynucleotide and an engineered protein. In some embodiments, the systems for use in the editing of a nucleic acid. In some embodiments, the systems cleave a target nucleic acid. In some embodiments, the systems generate a single strand break in the target nucleic acid. In some embodiments, the systems incorporate the donor nucleic acid into the target nucleic acid, thereby replacing an abnormal nucleic acid sequence relative to a reference sequence.

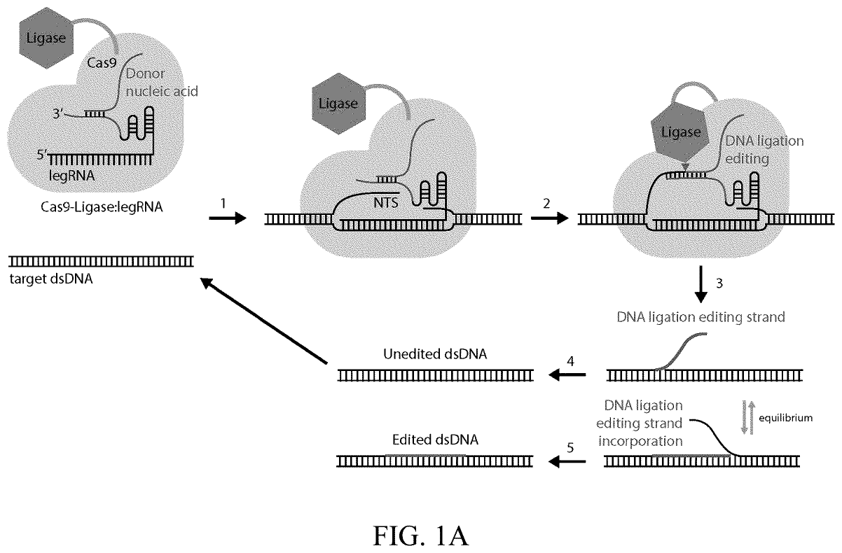

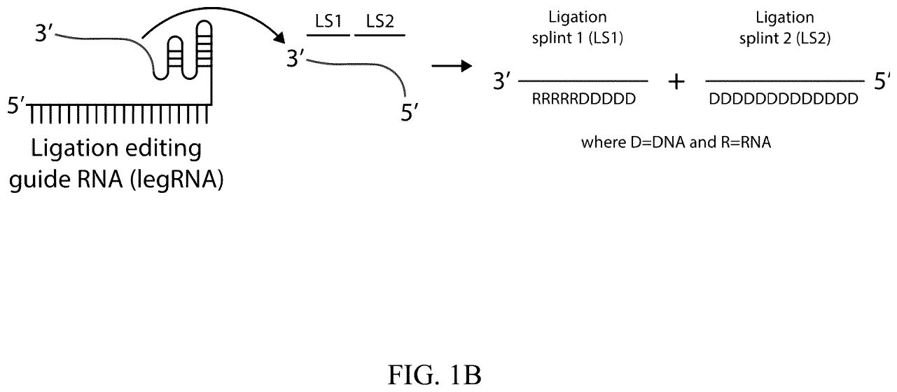

The systems provided herein include the following elements: (1) a donor nucleic acid; (2) a guide polynucleotide; (3) a nuclease, a nickase, or a variant thereof; and (4) a DNA ligase or a functional fragment thereof. An exemplary system is shown in FIG. 1A. Components of the system include an engineered protein that includes a of Cas9 nickase, a DNA ligase, a donor nucleic acid, and a guide polynucleotide that includes a DNA ligation splint complex to form a ribonucleoprotein (RNP). The DNA ligation splint can comprise a ligation splint 2 region provided herein and/or a ligation splint 1 region provided herein. The RNP can target double-stranded DNA (dsDNA) in the presence of a protospacer adjacent motif (PAM) and high sequence homology between the guide polynucleotide and the dsDNA. Following engagement between the Cas9-Ligase: guide polynucleotide complex and dsDNA, the Cas9 nickase creates a single-stranded break in the non-target strand (NTS) allowing the release of a single-stranded DNA (Step 1). The DNA ligation splint of the guide polynucleotide can then hybridize to this single-stranded DNA along with a ligation donor nucleic acid, presenting a substrate for DNA ligase to directly attach the 5′ end of the donor nucleic acid to the 3′ end of the NTS (Step 2). Following the DNA ligation editing step, the Cas9-Ligase: guide polynucleotide complex dissociates from the target dsDNA and this results in different conformations where the DNA ligation editing strand is either unhybridized or hybridized to the dsDNA (Step 3). Subsequently, endogenous cellular enzymes for DNA repair incorporate either the original DNA strand or newly-attached DNA ligation editing strand into the dsDNA, resulting in either an unedited dsDNA (Step 4) or edited dsDNA (Step 5), respectively.

In some arrangements, the guide polynucleotide for DNA editing by DNA ligase includes two elements—(1) the ligation splint 1 region (LS1) and (2) the ligation splint 2 region (LS2). The LS1 mediates hybridization with the non-target strand (NTS), whereas the LS2 mediates hybridization with the ligation donor oligonucleotide (FIG. 1B). Following the hybridization of LS1 and LS2 with the NTS and donor oligonucleotide, respectively, this presents a substrate for DNA ligase to directly attach the donor nucleic acid to the non-target strand (also referred to herein as the leading strand) through a DNA ligation event. The LS1 includes RNA (typically on the 3′ end) and DNA (typically on the 5′ end) at varying proportions to enhance DNA ligation efficiencies. The LS2 typically includes a majority DNA bases to facilitate efficient DNA ligation by DNA ligase. In some embodiments, the LS2 region comprises RNA only. In some embodiments, the LS1 region comprises DNA only.

The systems provided herein are designed to insert a nucleic acid with high fidelity and high processivity. The systems provided herein are useful in permitting precise, targeted insertion of polynucleotides into a target nucleic acid. In some embodiments, the systems provided herein permit targeted insertion of a polynucleotide that is at least 5, at least 10, at least 15, at least 20, at least 25, at least 30, at least 35, at least 40, at least 45, at least 50, at least 100, at least 200, at least 300, at least 400, at least 500, at least 600, at least 700, at least 800, at least 900, at least 1000, at least 10,000, at least 20,000, at least 30,000, at least 40,000, at least 50,000, at least 60,000, at least 70,000, at least 80,000, at least 90,000, at least 100,000 or more. In some embodiments, the systems provided herein permit targeted insertion of a single stranded polynucleotide that is 10 kb or larger.

### Donor Nucleic Acids.

Provided herein are compositions and systems comprising donor nucleic acids. A donor nucleic acid provides a high-fidelity DNA insertion to introduce a modification to the target nucleic acid and can further correct an aberration in the sequence relative to the wild-type sequence. In some embodiments, the wild-type sequence is a reference sequence, a nucleic acid sequence from a healthy subject, or a nucleic acid sequence from a healthy cell. In some embodiments, the donor nucleic acid comprises the reverse complement sequence of the target nucleic acid. In some embodiments, the donor nucleic acid comprises one or more alterations in a nucleobase, nucleoside, or nucleotide. In some embodiments, the donor nucleic acid comprises a reverse complement sequence of a sequence encoding an intron or a variant thereof. In some embodiments, the donor nucleic acid comprises a reverse complement sequence of a sequence encoding an exon or a variant thereof. In some embodiments, the donor nucleic acid comprises a reverse complement sequence of a sequence encoding for an exon and an intron. In some embodiments, the donor nucleic acid comprises a complement sequence of a sequence encoding an intron or a variant thereof. In some embodiments, the donor nucleic acid comprises a complement sequence of a sequence encoding an exon or a variant thereof. In some embodiments, the donor nucleic acid comprises a complement sequence of a sequence encoding for an exon and an intron. In some embodiments, the donor nucleic acid comprises a reverse complement sequence of a sequence encoding a non-coding element (e.g., promoter, enhancer) or a variant thereof. In some embodiments, the donor nucleic acid comprises a complement sequence of a sequence encoding a non-coding element (e.g., promoter, enhancer) or a variant thereof. In some embodiments, the donor nucleic acid comprises an alteration. In some embodiments, the donor nucleic acid comprises a sequence comprising at least one nucleobase that is complementary to or is mismatched with a sequence encoding a splice acceptor site. In some embodiments, the donor nucleic acid sequence comprises an A/C mismatch, an A/T mismatch, an A/G mismatch, a T/C mismatch, a T/G mismatch, a T/A mismatch, a C/G mismatch, a C/A mismatch, a C/T mismatch, a G/C mismatch, a G/T mismatch, a G/A mismatch, or any combination thereof relative to the target nucleic acid sequence or the complementary strand (also referred to as a lagging strand) provided herein. In some embodiments, the donor nucleic acid comprises an epigenetic alteration (e.g., methylation).

### Guide Polynucleotides

Provided herein are compositions and systems comprising a guide polynucleotide or a polynucleotide encoding the guide polynucleotide. A guide polynucleotide provided herein binds to a target nucleic acid and a nuclease or a nickase provided herein to form a complex. In some embodiments, the complex facilitates cleavage of the target nucleic acid.

In some embodiments, the guide polynucleotide comprises RNA nucleosides and DNA nucleosides. In some embodiments, the guide polynucleotide comprises RNA nucleotides and DNA nucleotides. In some embodiments, the guide polynucleotide comprises RNA nucleobases and DNA nucleobases. Examples of nucleobases include, but are not limited to, adenine (A), guanine (G), cytosine (C), thymine (T), and uracil (U). The nucleobase of a nucleotide can be independently selected from a purine, a pyrimidine, a purine or pyrimidine analog. In an embodiment, the nucleobase can include, for example, naturally-occurring and synthetic derivatives of a base. Guide polynucleotides provided herein can comprise non-naturally occurring sequences or engineered sequences. In some embodiments, a polynucleotide encoding the guide polynucleotide provided herein comprises a promoter region or a localization sequence. For example, a U6 promoter can be used to drive the expression of the guide polynucleotide in a cell.

In some embodiments, the guide polynucleotide provided herein or the donor nucleic acid provided herein comprise a modified nucleobase, a modified nucleotide, or a modified nucleoside. The modified nucleosides and modified nucleotides described herein, which can be incorporated into a target nucleic acid, can include a modified nucleobase. In some embodiments, the nucleobase or the nucleotide provided herein is chemically modified. Nucleobases can be modified or replaced to provide modified nucleosides and modified nucleotides that can be incorporated into a target nucleic acid.

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m C), N4-acetyl-cytidine (act), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k C), a-thio-cytidine, 2′-0-methyl-cytidine (Cm), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f 5 Cm), N4,N4,2′-0-trimethyl-cytidine (m4 2 Cm), 1-thio-cytidine, 2′-F-ara-cytidine, 2′-F-cytidine, and 2′-OH-ara-cytidine, 2′-OMe-exNA-cytidine, and 2′-F-exNA-cytidine.

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (i A), 2-methyl-adenine (m2A), N6-methyl-adenosine (m6A), 2-methylthio-N6-methyl-adenosine (ms2m6A), N6-isopentenyl-adenosine (16A), 2-methylthio-N6-isopentenyl-adenosine (ms216A), N6-(cis-hydroxyisopentenyl) adenosine (io6A), 2-methylthio-N6-(cis-hydroxyisopentenyl) adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m6 2A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, a-thio-adenosine, 2′-0-methyl-adenosine (Am), N6,2′-0-dimethyl-adenosine (m6Am), N6-Methyl-2′-deoxyadenosine, N6,N6,2′-0-trimethyl-adenosine (m6 2Am), 1,2′-0-dimethyl-adenosine (i Am), 2′-0-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, N6-(19-amino-pentaoxanonadecyl)-adeno sine, 2′-OMe-exNA-adenosine, and 2′-F-exNA-adenosine.

In some embodiments, the modified nucleobase is a modified guanine. Exemplary nucleobases and nucleosides having a modified guanine include without limitation inosine (I), 1-methyl-inosine (iVl), wyosine (imG), methylwyosine (mimG), 4-demethyl-wyosine (imG-14), isowyosine (imG2), wybutosine (yW), peroxywybutosine (o2yW), hydroxy wybuto sine (OHyW), undermodified hydroxy wybuto sine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQO, archaeosine (G+), 7-deaza-8-aza-guanosine, 6-thio-guanosine, 6-thio-7-deaza-guanosine, 6-thio-7-deaza-8-aza-guanosine, 7-methyl-guanosine (m G), 6-thio-7-methyl-guanosine, 7-methyl-inosine, 6-methoxy-guanosine, 1-methyl-guanosine (m′G), N2-methyl-guanosine (m 2 G), N2,N2-dimethyl-guanosine (m 2 2G), N2,7-dimethyl-guanosine (m 2,7G), N2, N2,7-dimethyl-guanosine (m 2,2,7G), 8-oxo-guanosine, 7-methyl-8-oxo-guanosine, 1-meth thio-guanosine, N2-methyl-6-thio-guanosine, N2,N2-dimethyl-6-thio-guanosine, a-thio-guanosine, 2′-0-methyl-guanosine (Gm), N2-methyl-2′-0-methyl-guanosine (m 2″Gm), N2,N2-dimethyl-2′-0-methyl-guano sine (m 2 2Gm), 1-methyl-2′-0-methyl-guanosine (m′Gm), N2,7-dimethyl-2′-0-methyl-guanosine (m″,7Gm), 2′-0-methyl-inosine (Im), 1,2′-0-dimethyl-inosine (m′lm), 06-phenyl-2′-deoxyinosine, 2′-0-ribosylguanosine (phosphate) (Gr(p)), 1-thio-guanosine, 06-methyl-guanosine, 06-Methyl-2′-deoxy guanosine, Z-F-ara-guanosine, 2′-F-guanosine, 2′-OMe-exNA-guanosine, and 2′-F-exNA-guanosine.

In some embodiments, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m 3 U), 5-methoxy-uridine (mo 5 U), uridine 5-oxyacetic acid (cmo 5 U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm 5 se 2 U), 5-carbamoylmethyl-uridine (ncm 5 U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (xcm5U), 1-taurinomethyl-pseudouridine, 5-taurinomethyl-2-thio-uridine (Tm5s2U), 1-taurinomethyl-4-thio-pseudouridine, 5-methyl-uridine (m5U, i.e., having the nucleobase deoxythymine), 1-methyl-pseudouridine (n′y), 5-methyl-2-thio-uridine (m5s2U), 1-methyl-4-thio-pseudouridine (m xi/), 4-thio-1-methyl-pseudouridine, 3-methyl-pseudouridine (m), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N 1-methyl-pseudouridine, 3-(3-amino-3-carboxypropyl) uridine (acp 3 U), 1-methyl-3-(3-amino-3-carboxypropyl) pseudouridine (acp 3 ψ), 5-(isopentenylaminomethyl) uridine (inm5U), 5-(isopentenylaminomethyl)-2-thio-uridine (inm5s2U), a-thio-uridine, 2′-0-methyl-uridine (Um), 5,2′-0-dimethyl-uridine (m5Um), 2′-0-methyl-pseudouridine (ψηι), 2-thio-2′-0-methyl-uridine (s2Um), 5-methoxycarbonylmethyl-2′-O-methyl-uridine (mem 5Um), 5-carbamoylmethyl-2′-0-methyl-uridine (ncm 5Um), 5-carboxymethylaminomethyl-2′-0-methyl-uridine (cmnm 5 Um), 3,2′-0-dimethyl-uridine (m 3 Um), 5-(isopentenylaminomethyl)-2′-0-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, pyrazolo[3,4-d]pyrimidines, xanthine, hypoxanthine, 2′-OMe-exNA-uridine, and 2′-F-exNA-uridine.

In some embodiments, the modified nucleobase is a modified thymine. In some embodiments, the modified nucleoside is a modified thymidine. Non-limiting examples of modified thymine and thymidine include: 6-(azo)thymine, 3′-azido-3′-deoxythymidine, 2′,3′-didehydro-2′,3′-dideoxythymidine; 1-(2,3-dideoxy-beta-D-glyceropent-2-enofuranosyl)thymine, 3-(2-chloroethyl)thymidine, 3′-fluoro-3′-deoxythymidine, β-L-2′-deoxythymidine, thieno[3,4-d]-pyrimidine T-mimic deoxynucleoside, 1-(2-Deoxy-β-D-threo-pentofuranosyl)thymine, 5-ethynyl-2′-deoxyuridine, bromodeoxyuridine, tritiated thymidine, 5-chlorodeoxyuridine (CldU), 5-iododeoxyuridine (IdU), 2-thiothymidine triphosphate, 5-(α-tert-butylortho-bromobenzyloxy) methyl-2′-deoxyuridine, 5-(α-methylbenzyloxy)methyluracil, and 5-ethyldeoxyuridine.

Nucleic acids can be modified using various chemistries and modifications. In some embodiments, regular internucleosidic linkages between nucleotides can be altered by mono- or di-thiolation of the phosphodiester bonds to yield phosphorothioate esters or phosphorodithioate esters, respectively. Other modifications of the internucleosidic linkages can include amidation or peptide linkers. A ribose sugar can be modified by substitution of the 2′-O moiety with a lower alkyl (C1-4, such as 2′-O-Me), alkenyl (C2-4), alkynyl (C2-4), methoxyethyl(2′-MOE), or other substituent. In some cases, substituents of the 2′ OH group can comprise a methyl, methoxyethyl or 3,3′-dimethylallyl group. In some cases, locked nucleic acid sequences (LNAs), comprising a 2′-4′ intramolecular bridge (such as a methylene bridge between the 2′ oxygen and 4′ carbon) linkage inside the ribose ring, can be applied. Purine nucleobases and/or pyrimidine nucleobases can be modified to alter their properties, for example by amination or deamination of the heterocyclic rings. Many of these modified nucleobases and their corresponding ribonucleosides are available from commercial suppliers. If desired, the guide polynucleotides and/or donor nucleic acids can contain phosphoramidate, phosphorothioate, and/or methylphosphonate linkages. Several suitable methods can be used to produce nucleic acid molecules and nucleic acids containing modified nucleobases. For example, a guide polynucleotide and/or a donor nucleic acid that contains modified nucleotides can be prepared by transcribing a DNA that encodes for the guide polynucleotide and/or the donor nucleic acid using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. In some embodiments, the guide polynucleotide and/or the donor nucleic acid that contains modified nucleotides can be prepared by in vitro transcription. The transcription reaction can contain nucleotides and modified nucleotides, and other components that support the activity of the selected polymerase, such as a suitable buffer, and suitable salts. The incorporation of nucleotide analogs into a guide polynucleotides may be engineered, for example, to alter the stability of such RNA/DNA molecules or to increase resistance against RNases.

In some embodiments, the guide polynucleotide comprises a secondary structure or a tertiary structure. In some embodiments, the secondary structure comprises a bulge, a stem, a stem loop, a loop, a tetraloop, a hairpin, a wobble base pair, a pseudoknot, a nexus, or a combination thereof. In some embodiments, the bulge, the stem loop, or the hairpin comprise an unpaired region of nucleotides within a nucleic acid duplex.

In some embodiments, the guide polynucleotides provided herein comprise a targeting region that has complementarity to a target nucleic acid. In some embodiments, the targeting region comprises RNA. In some embodiments, the targeting region has at least 80% complementarity to the target nucleic acid. In some embodiments, the targeting region has at least 85% complementarity to the target nucleic acid. In some embodiments, the targeting region has at least 90% complementarity to the target nucleic acid. In some embodiments, the targeting region has at least 95% complementarity to the target nucleic acid. In some embodiments, the targeting region has at least 99% complementarity to the target nucleic acid. In some embodiments, the targeting region has 100% complementarity to the target nucleic acid.

In some embodiments, the targeting region comprises at least 10 ribonucleotides, at least 15 ribonucleotides, at least 20 ribonucleotides, at least 25 ribonucleotides, at least 30 ribonucleotides, at least 35 ribonucleotides, at least 40 ribonucleotides, at least 45 ribonucleotides, at least 50 ribonucleotides or more. In some embodiments, the targeting region comprises at least about 10 up to 15 ribonucleotides for enhanced specificity. In some embodiments, the targeting region comprises at least about 20 to 30 ribonucleotides for enhanced structural stability and specificity.

In some embodiments, the targeting region hybridizes to a target nucleic acid. In some embodiments, a nuclease or a nickase cleaves the target nucleic acid to generate a leading strand and a complementary strand (also referred to herein as a lagging strand). In some embodiments, upon cleavage of the target nucleic acid by an engineered protein construct provided herein or a nickase, the targeting region binds to at least a portion of the cleaved target nucleic acid. In some embodiments, the targeting region binds to the complementary strand. In some embodiments, the complementary strand comprises a protospacer adjacent motif (PAM). A PAM is a short nucleic acid sequence (usually 2-6 base pairs in length) that precedes the region targeted for cleavage by the nuclease or the nickase provided herein. In some embodiments, the targeting region binds to a target gene sequence that is within at least 20 nucleotides, at least 15 nucleotides, at least 10 nucleotides, or at least 5 nucleotides from a protospacer adjacent motif (PAM). The guide polynucleotide can bind upstream 5′ of a protospacer adjacent motif (PAM) sequence or downstream 3′ of a PAM sequence via the targeting region.

In some embodiments, the guide polynucleotides comprise a protein binding region. In some embodiments, the protein binding region comprises a secondary structure that binds to a protein. In some embodiments, the protein binding region binds to a nuclease, a nickase, an endonuclease, or an exonuclease. In some embodiments, the protein binding region binds to a Cas protein. The secondary structure can minimize the potential of the guide polynucleotide to interfere with nuclease or nickase activity when the guide polynucleotide is in complex with the an engineered protein provided herein. In some embodiments, the secondary structure comprises: a bulge, a stem, a loop, a hairpin, a wobble base pair, a pseudoknot, or a combination thereof.

In some embodiments, the guide polynucleotides comprise a ligation splint 2 (LS2) region. In some embodiments, the ligation splint 2 region is complementary to the donor nucleic acid. In some embodiments, the ligation splint 2 region has complementarity to the target nucleic acid. In some embodiments, the LS2 region is 5′ of the LS1 region. In some embodiments, the LS2 region is within the protein-binding region. In some embodiments, the LS2 region comprises deoxyribonucleotides. In some embodiments, the LS2 region is within the protein-binding region of the guide polynucleotide. In some embodiments, the LS2 region comprises secondary or tertiary structure that enhances the stability of the guide polynucleotide. In some embodiments, the ligation splint 2 region comprises at least one mismatch nucleobase relative to the target nucleic acid. In some embodiments, the LS2 region comprises at least about 3 nucleobases up to 100,000 nucleobases in length. In some embodiments, the ligation splint 2 region comprises at least about 5 nucleobases up to 10,000 nucleobases. In some embodiments, the ligation splint 2 region comprises at least about 7 nucleobases up to 10,000 nucleobases.

In some embodiments, the ligation splint 2 region comprises the reverse complement sequence of the target nucleic acid or a strand thereof. The LS2 region and/or the donor nucleic acid can be designed to correct an aberration in a target sequence relative to the wild-type sequence. For example, a wild-type sequence can include a reference sequence, a nucleic acid sequence from a healthy subject, or a nucleic acid sequence from a healthy cell. Methods of obtaining a reference sequence for a polynucleotide can include sequencing or sequence alignment tools and databases such as NCBI BLAST or UniProt.

In some embodiments, the ligation splint 2 region comprises one or more alterations in a nucleobase, nucleoside, or nucleotide. In some embodiments, the ligation splint 2 region comprises a reverse complement sequence of a sequence encoding an intron or a variant thereof. In some embodiments, the ligation splint 2 region comprises a reverse complement sequence of a sequence encoding an exon or a variant thereof. In some embodiments, the ligation splint 2 region comprises a reverse complement sequence of a sequence encoding for an exon and an intron. In some embodiments, the ligation splint 2 region comprises a complement sequence of a sequence encoding an intron or a variant thereof. In some embodiments, the ligation splint 2 region comprises a complement sequence of a sequence encoding an exon or a variant thereof. In some embodiments, the ligation splint 2 region comprises a complement sequence of a sequence encoding for an exon and an intron. In some embodiments, the ligation splint 2 region comprises a reverse complement sequence of a sequence encoding a non-coding element (e.g., promoter, enhancer) or a variant thereof. In some embodiments, the ligation splint 2 region comprises a complement sequence of a sequence encoding a non-coding element (e.g., promoter, enhancer) or a variant thereof.

In some embodiments, a guide polynucleotide provided herein comprises one or more alterations. In some embodiments, the one or more alterations comprise a change in at least one nucleobase, nucleoside, or nucleotide of the LS2 or the LS1 region. In some embodiments, the ligation splint 2 region comprises a sequence comprising at least one nucleobase that is complementary to or is mismatched with a sequence encoding a splice acceptor site. In some embodiments, the ligation splint 2 region comprises an A/C mismatch, an A/T mismatch, an A/G mismatch, a T/C mismatch, a T/G mismatch, a T/A mismatch, a C/G mismatch, a C/A mismatch, a C/T mismatch, a G/C mismatch, a G/T mismatch, a G/A mismatch, or any combination thereof relative to the target nucleic acid sequence or the complementary strand (also referred to as a lagging strand) provided herein.

In some embodiments, guide polynucleotides provided herein comprise a ligation splint 1 region. In some embodiments, the ligation splint 1 region comprises a ribonucleotide, ribonucleotides, or an RNA region. In some embodiments, the ligation splint 1 region comprises a deoxyribonucleotide, deoxyribonucleotides, or a DNA region. In some embodiments, the ligation splint 1 region comprises ribonucleotides and deoxyribonucleotides. In some embodiments, the ligation splint 1 region comprises at least one ribonucleoside and at least one deoxyribonucleoside.

In some embodiments, the LS1 region of the guide polynucleotide forms a DNA-RNA (DR)-loop upon association with a target nucleic acid. The DR-loop provides structural framework and stability for the formation of the complex between the target nucleic acid, the guide polynucleotide, and the engineered protein provided herein. In some embodiments, the LS1 forms a DR-loop upon association with a target nucleic acid and an engineered protein provided herein.

In some embodiments, the LS1 region forms a DNA (D)-loop upon association with a target nucleic acid. A D loop forms following nickase cleavage of the target DNA. The D-loop is a DNA structure where the two strands of a double-stranded DNA molecule are separated for a stretch and held apart by a third strand of DNA. In some embodiments, the third strand of DNA in the D-loop is the LS2 region or the LS1 region of the guide polynucleotide.

In some embodiments, the LS1 region forms an R-loop upon association with a target nucleic acid. In some embodiments, the LS1 region forms an RNA (R)-loop upon association with a target nucleic acid and an engineered protein provided herein. The R-loop can form following nickase cleavage of the target nucleic acid between the complementary strand of the target DNA, the RNA portion of the LS1 region of the guide polynucleotide, and the leading strand of the target DNA.

In some embodiments, the ligation splint 1 region hybridizes to a leading strand of a DNA upon cleavage of the target nucleic acid by an engineered protein construct provided herein. In some embodiments, the ribonucleosides, the ribonucleotides, or the RNA region within the ligation splint 1 region hybridizes to the leading strand. In some embodiments, the LS1 region is 3′ from the LS2 region. In some embodiments, the LS1 region is within the protein binding region.

In some embodiments, the ligation splint 1 region comprises a ratio of ribonucleic acids (RNA) to deoxyribonucleic acids (DNA) of: 1:1 up to 20:1. In some embodiments, the ligation splint 1 region comprises a ratio of ribonucleic acids (RNA) to deoxyribonucleic acids (DNA) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, or 20:1. In some embodiments, the ligation splint 1 region comprises at least about 3 nucleobases up to about 20 nucleobases.

The interaction between the donor nucleic acid, guide polynucleotide provided herein, and an engineered protein provided herein is illustrated in FIG. 1A. An exemplary guide polynucleotide structure is shown in FIG. 1B. Thus, in some embodiments, the guide polynucleotides provided herein comprise in a 5′ to 3′ direction: (i) a targeting region that has complementarity to a target nucleic acid; (ii) a protein binding region, wherein the protein binding region comprises a secondary structure that binds to the nickase region, (iii) a ligation splint 2 region that has complementarity to the donor nucleic acid, the target nucleic acid and at least one mismatch nucleobase relative to the target nucleic acid; and (iv) a ligation splint 1 region, wherein the ligation splint 1 region comprises: a DNA nucleoside and an RNA nucleoside, wherein the ligation splint 1 region has complementarity to the target nucleic acid. In some embodiments, upon introduction to a cell or a cell-free system, the compositions or systems provided herein incorporates the donor nucleic acid into the target nucleic acid, thereby modifying the target nucleic acid. In some embodiments, the newly inserted nucleic acid comprises a single-stranded DNA sequence that is substantially complementary to the complementary strand (that comprises a PAM sequence) and also comprises at least one mismatched nucleobase relative to the complementary strand. In some embodiments, the ligation splint 1 region forms an R-loop upon association with a target nucleic acid and a DNA ligase.

In some embodiments, the guide polynucleotides comprise a sequence or a portion of a sequence in Table 3 or Table 7. In some embodiments, the guide polynucleotides comprise a sequence that is at least 75% identical, 80% identical, 85% identical, 90% identical, 95% identical, 99% identical, or 100% identical to any one of SEQ ID NOS: 45 to 57 or SEQ ID NOS: 102 to 123.

The guide polynucleotides provided herein can be produced, for example, by phosphoramidite chemical synthesis, in-vitro transcription (IVT) techniques, M13 bacteriophage methods, DNA isolation, RNA isolation, tagmentation, and combinations thereof. Furthermore, guide polynucleotides can be generated and expressed by transducing or transfecting cells with a plasmid DNA or a vector that comprises a polynucleotide encoding for an expression cassette.

In some embodiments, the guide polynucleotides comprise a 5′ cap. Free 5′ hydroxyl groups can be capped by acetylation. In some embodiments, the guide polynucleotides comprise a 3′ polyadenylated tail. In some embodiments, the guide polynucleotides comprise a polynucleotide linker or spacer regions.

The engineered proteins that interact with the guide polynucleotides provided herein can be used in the systems and compositions provided herein to insert a nucleic acid. Engineered proteins useful in the editing of nucleic acids and targeting cleavage of a particular target sequence are described in further detail below.

### Engineered Proteins

Provided herein are engineered proteins that specifically bind to a target nucleic acid using the guide polynucleotide to hybridize to the target sequence or polynucleotides encoding the engineered proteins provided herein. The engineered proteins also ligate a nucleic acid donor oligonucleotide for incorporation into the target nucleic acid. The engineered proteins provided herein can be fused to one or more additional protein constructs for a particular application. In some embodiments, a particular application is DNA ligation or cleavage of a nucleic acid. Multimerization of an engineered protein construct provided herein can be achieved by direct fusion with another engineered protein construct. In some embodiments, each protein construct is operably linked by a linker polypeptide.

Provided herein are systems, compositions, and engineered proteins that comprise a nuclease or a nickase region. A nuclease is an enzyme that cleaves a nucleic acid. For example, a nuclease can create a single or a double-stranded break in a target nucleic acid. Nucleases and nickases target specific nucleic acid sequences by binding to a guide polynucleotide that hybridizes to the target nucleic acid. Various types of nucleases can be used in the systems and compositions provided herein.

Provided herein are systems, compositions, and engineered proteins that comprise a nickase or a nickase region. In some embodiments, the engineered proteins comprise a nickase. In some embodiment the engineered proteins provided herein comprise: an engineered protein construct comprising a nickase region. A nickase is an enzyme that cuts one strand of a double-stranded DNA.

In some embodiments, the nickase provided herein cut one strand of the DNA duplex to produce DNA molecules that are nicked. Exemplary amino acid sequences for nucleases and nickases, include but are not limited to, for example, NCBI Gene ID: 1238121: NP 858382.1 [conjugal transfer nickase/helicase Tral (plasmid) [Shigella flexneri 2a str. 301]]

(SEQ ID NO: 67)
MKAGEESVAQVSGVREQAILTQAIRSELKTQGVLGHPEVTMTALSPVWLDSRSRYLRDMYRPGMVMEQWNPETRSHDR
YVTERVTAQSHSLTLRNAQGETQVVRISSLDSSWSLFRPEKMPVADGERLRVTGKIPGLRVSGGDRLQVASVSEDAMT
VVVPGRAEPATLPVSDSPFTALKLENGWVETPGHSVSDSATVFASVTQMAMDNATLNGLARSGRDVRLYSSLDETRTA
EKLARHPSFTVVSEQIKARAGETLLETAISLQKAGLHTPAQQAIHLALPVLESKNLAFSMVDLLTEAKSFAAEGTGFA
DLGGEINAQIKRGDLLYVDVAKGYGTGLLVSRASYEAEKSILRHILEGKEAVTPLMERVPGELMEKLTSGQRAATRMI
LETSDRFTVVQGYAGVGKTTQFRAVMSAVNMLPESERPRVVGLGPTHRAVGEMRSAGVDAQTLASFLHDTQLQQRSGE
TPDFSNTLFLLDESSMVGNTDMARAYALIAAGGGRAVASGDTDQLQAIAPGQPFRLQQTRSAADVVIMKEIVRQTPEL
REAVYSLINRDVERALSGLERVKPSQVPRLEGAWAPEHSVTEFSHSQEAKLAEAQQKAMLKGEAFPDVPMTLYEAIVR
DYTGRTPEAREQTLIVTHLNEDRRVLNSMIHDAREKAGELGKVQVMVPVLNTANIRDGELRRLSTWENNPDALALVDN
VYHRIAGISKDDGLITLQDAEGNTRLISPREAVAEGVTLYTPDTIRVGTGDRIRFTKSDRERGYVANSVWTVTAVSGD
SVTLSDGQQTRVIRPGQERAEQHIDLAYAITAHGAQGASETFAIALEGTEGNRKLMAGFESAYVALSRMKQHVQVYTD
NRQGWTDAINNAVQKGTAHDVFEPKPDREVMNAERLFSTARELRDVAAGRAVLRQAGLAGGDSPARFIAPGRKYPQPY
VALPAFDRNGKSAGIWLNPLTTDDGNGLRGFSGEGRVKGSGDAQFVALQGSRNGESLLADNMQDGVRIARDNPDSGVV
VRIAGEGRPWNPGAITGGRVWGDIPDNSVQPGAGNGEPVTAEVLAQRQAEEAIRRETERRADEIVRKMAENKPDLPDG
KTEQAVREIAGQERDRAAITEREAALPESVLREPQRVREAVREVARENLLQERLQQMERDMVRDLQKEKTPGGD;
UniProt: Q8YS92 [DNA Nickase-Nostoc sp. (strain PCC 7120/SAG 25.82/UTEX 2576)]:
(SEQ ID NO: 68)
MVSTLDDTKRNAIAEKLADAKLLQELIIENQERFLRESTDNEISNRIRDFLEDDRKNLGIIETVIVQYGIQKEPRQTV
REMVDQVRQLMQGSQLNFFEKVAQHELLKHKQVMSGLLVHKAAQKVGADVLAAIGPLNTVNFENRAHQEQLKGILEIL
GVRELTGQDADQGIWGRVQDAIAAFSGAVGSAVTQGSDKQDMNIQDVIRMDHNKVNILFTELQQSNDPQKIQEYFGQI
YKDLTAHAEAEEEVLYPRVRSFYGEGDTQELYDEQSEMKRLLEQIKAISPSAPEFKDRVRQLADIVMDHVRQEESTLF
AAIRNNLSSEQTEQWATEFKAAKSKIQQRLGGQATGAGV;
Streptococcus pyogenes Cas9:
(SEQ ID NO: 69)
MDKKYSIGLDIGTNSVGWAVITDEYKVPSKKFKVLGNTDRHSIKKNLIGALLEDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFYKFIKPILEKMDGTEELL
VKLNREDLLRKQRTEDNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVTEGMRKPAFLSGEQKK
AIVDLLFKTNRKVTVKQLKEDYFKKIECEDSVEISGVEDRENASLGTYHDLLKIIKDKDELDNEENEDILEDIVLTLT
LFEDREMIEERLKTYAHLEDDKVMKQLKRRRYTGWGRLSRKLINGIRDKQSGKTILDFLKSDGFANRNFMQLIHDDSL
TEKEDIQKAQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQSFLKDDSIDNKVLT
RSDKNRGKSDNVPSEEVVKKMKNYWRQLLNAKLITQRKFDNLTKAERGGLSELDKAGFIKRQLVETRQITKHVAQILD
SRMNTKYDENDKLIREVKVITLKSKLVSDERKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKGRDFATVRKVLSMPQV
NIVKKTEVQTGGESKESILPKRNSDKLIARKKDWDPKKYGGEDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGD
Streptococcus pyogenes Cas9 H840A nickase (bold/underlined indicates amino acid
substitution):
(SEQ ID NO: 70)
MDKKYSIGLDIGTNSVGWAVITDEYKVPSKKFKVLGNTDRHSIKKNLIGALLEDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKSRRLENLIAQLPGEKK
NGLFGNLIALSLGLTPNFKSNEDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFYKFIKPILEKMDGTEELL
VKLNREDLLRKQRTEDNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRENASLGTYHDLLKIIKDKDFLDNEENEDILEDIVLTLT
LFEDREMIEERLKTYAHLEDDKVMKQLKRRRYTGWGRLSRKLINGIRDKQSGKTILDELKSDGFANRNFMQLIHDDSL
TFKEDIQKAQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQSFLKDDSIDNKVLT
RSDKNRGKSDNVPSEEVVKKMKNYWRQLLNAKLITQRKFDNLTKAERGGLSELDKAGFIKRQLVETRQITKHVAQILD
SRMNTKYDENDKLIREVKVITLKSKLVSDERKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGEDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGD;
Streptococcus pyogenes Cas9 R221K, N394K, H840A nickase (bold/underlined indicates
amino acid substitution):
(SEQ ID NO: 71)
MDKKYSIGLDIGTNSVGWAVITDEYKVPSKKFKVLGNTDRHSIKKNLIGALLFDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKSRKLENLIAQLPGEKK
NGLFGNLIALSLGLTPNEKSNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFYKFIKPILEKMDGTEELL
VKLKREDLLRKQRTEDNGSIPHQIHLGELHAILRRQEDFYPELKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRENASLGTYHDLLKIIKDKDELDNEENEDILEDIVLTLT
LFEDREMIEERLKTYAHLEDDKVMKQLKRRRYTGWGRLSRKLINGIRDKQSGKTILDELKSDGFANRNEMQLIHDDSL
TFKEDIQKAQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQSFLKDDSIDNKVLT
RSDKNRGKSDNVPSEEVVKKMKNYWRQLLNAKLITQRKEDNITKAERGGLSELDKAGFIKRQLVETRQITKHVAQILD
SRMNTKYDENDKLIREVKVITLKSKLVSDERKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHLFTLINLGAPAAFKY
FDTTIDRKRYTSTKEVLDATLIHQSITGLYETRIDLSQLGGD;
Staphylococcus aureus Cas9 N580A nickase (bold/underlined indicates amino acid
substitution):
(SEQ ID NO: 72)
MKRNYILGLDIGITSVGYGIIDYETRDVIDAGVRLFKEANVENNEGRRSKRGARRLKRRRRHRIQRVKKLLEDYNLLT
DHSELSGINPYEARVKGLSQKLSEEEFSAALLHLAKRRGVHNVNEVEEDTGNELSTKEQISRNSKALEEKYVAELQLE
RLKKDGEVRGSINRFKTSDYVKEAKQLLKVQKAYHQLDQSFIDTYIDLLETRRTYYEGPGEGSPFGWKDIKEWYEMLM
GHCTYFPEELRSVKYAYNADLYNALNDLNNLVITRDENEKLEYYEKFQIIENVFKQKKKPTLKQIAKEILVNEEDIKG
YRVTSTGKPEFTNLKVYHDIKDITARKEIIENAELLDQIAKILTIYQSSEDIQEELTNLNSELTQEEIEQISNLKGYT
GTHNLSLKAINLILDELWHINDNQIAIFNRLKLVPKKVDLSQQKEIPTTLVDDFILSPVVKRSFIQSIKVINAIIKKY
GLPNDIIIELAREKNSKDAQKMINEMQKRNRQTNERIEEIIRTTGKENAKYLIEKIKLHDMQEGKCLYSLEAIPLEDL
LNNPFNYEVDHIIPRSVSFDNSFNNKVLVKQEEASKKGNRTPFQYLSSSDSKISYETFKKHILNLAKGKGRISKTKKE
YLLEERDINRFSVQKDFINRNLVDTRYATRGLMNLLRSYFRVNNLDVKVKSINGGFTSFLRRKWKFKKERNKGYKHHA
EDALIIANADFIFKEWKKLDKAKKVMENQMFEEKQAESMPEIETEQEYKEIFITPHQIKHIKDFKDYKYSHRVDKKPN
RELINDTLYSTRKDDKGNTLIVNNINGLYDKDNDKLKKLINKSPEKLLMYHHDPQTYQKLKLIMEQYGDEKNPLYKYY
EETGNYLTKYSKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDITYREYLENMNDKRPP
RIIKTIASKTQSIKKYSTDILGNLYEVKSKKHPQIIKKG;
and
Francisella novicida Cas9 H969A nickase (bold/underlined indicates amino acid
substitution):
(SEQ ID NO: 73)
MNFKILPIAIDLGVKNTGVFSAFYQKGTSLERLDNKNGKVYELSKDSYTLLMNNRTARRHQRRGIDRKQLVKRLFKLI
WTEQLNLEWDKDTQQAISFLENRRGFSFITDGYSPEYLNIVPEQVKAILMDIFDDYNGEDDLDSYLKLATEQESKISE
IYNKLMQKILEFKLMKLCTDIKDDKVSTKTLKEITSYEFELLADYLANYSESLKTQKFSYTDKQGNLKELSYYHHDKY
NIQEFLKRHATINDRILDTLLTDDLDIWNFNFEKEDEDKNEEKLQNQEDKDHIQAHLHHFVFAVNKIKSEMASGGRHR
SQYFQEITNVLDENNHQEGYLKNFCENLHNKKYSNLSVKNLVNLIGNLSNLELKPLRKYENDKIHAKADHWDEQKFTE
TYCHWILGEWRVGVKDQDKKDGAKYSYKDLCNELKQKVTKAGLVDELLELDPCRTIPPYLDNNNRKPPKCQSLILNPK
FLDNQYPNWQQYLQELKKLQSIQNYLDSFETDLKVLKSSKDQPYFVEYKSSNQQIASGQRDYKDLDARILQFIFDRVK
ASDELLLNEIYFQAKKLKQKASSELEKLESSKKLDEVIANSQLSQILKSQHTNGIFEQGTFLHLVCKYYKQRQRARDS
RLYIMPEYRYDKKLHKYNNTGREDDDNQLLTYCNHKPRQKRYQLLNDLAGVLQVSPNELKDKIGSDDDLFISKWLVEH
IRGFKKACEDSLKIQKDNRGLLNHKINIARNTKGKCEKEIFNLICKIEGSEDKKGNYKHGLAYELGVLLFGEPNEASK
PEFDRKIKKENSIYSFAQIQQIAFAERKGNANTCAVCSADNAHRMQQIKITEPVEDNKDKIILSAKAQRLPAIPTRIV
DGAVKKMATILAKNIVDDNWQNIKQVLSAKHQLHIPIITESNAFEFEPALADVKGKSLKDRRKKALERISPENIFKDK
NNRIKEFAKGISAYSGANLTDGDFDGAKEELDAIIPRSHKKYGTLNDEANLICVTRGDNKNKGNRIFCLRDLADNYKL
KQFETTDDLEIEKKIADTIWDANKKDFKFGNYRSFINLTPQEQKAFRHALFLADENPIKQAVIRAINNRNRTFVNGTQ
RYFAEVLANNIYLRAKKENLNTDKISFDYFGIPTIGNGRGIAEIRQLYEKVDSDIQAYAKGDKPQASYSHLIDAMLAF
CIAADEHRNDGSIGLEIDKNYSLYPLDKNTGEVFTKDIFSQIKITDNEFSDKKLVRKKAIEGENTHRQMTRDGIYAEN
YLPILIHKELNEVRKGYTWKNSEEIKIFKGKKYDIQQLNNLVYCLKFVDKPISIDIQISTLEELRNILTTNNIAATAE
YYYINLKTQKLHEYYIENYNTALGYKKYSKEMEFLRSLAYRSERVKIKSIDDVKQVLDKDSNFIIGKITLPFKKEWQR
LYREWQNTTIKDDYEFLKSFFNVKSITKLHKKVRKDESLPISTNEGKFLVKRKTWDNNFIYQILNDSDSRADGTKPFI
PAFDISKNEIVEAIIDSFTSKNIFWLPKNIELQKVDNKNIFAIDTSKWFEVETPSDLRDIGIATIQYKIDNNSRPKVR
VKLDYVIDDDSKINYFMNHSLLKSRYPDKVLEILKQSTIIEFESSGENKTIKEMLGMKLAGIYNETSNN.

In some embodiments, a system or an engineered protein provided herein comprises a sequence that is at least 85% identical to any one of SEQ ID NOS: 67-73. In some embodiments, a system or an engineered protein provided herein comprises a sequence that is at least 90% identical to any one of SEQ ID NOS: 67-73. In some embodiments, a system or an engineered protein provided herein comprises a sequence that is at least 95% identical to any one of SEQ ID NOS: 67-73. In some embodiments, a system or an engineered protein provided herein comprises a sequence that is at least 99% identical to any one of SEQ ID NOS: 67-73. In some embodiments, a system or an engineered protein provided herein comprises any one of SEQ ID NOS: 67-73.

In some embodiments, the engineered proteins comprise a Cas protein or a fragment thereof. In some embodiments, the engineered proteins comprise a Cas protein or a fragment thereof that comprise at least one amino acid substitution relative to a reference amino acid sequence for the Cas protein of the fragment thereof (e.g., the wild-type sequence). In some embodiments, the Cas is a catalytically dead Cas or a partially dead Cas. For example, a partially dead Cas can include a nickase. In some embodiments, the catalytically dead Cas or the partially dead Cas is selected from the group consisting of catalytically dead derivatives of: Cas1, Cas1B, Cas2, Cas3, Cas4, Cas5, Cas6, Cas7, Cas8, Cas9, Cas10, Cas 11, Cas 12a (Cpf1), Cas 12b, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, c2c1, c2c3, Cas9HiFi, xCas9, CasX, CasY, CasRX, SpCas9-VQR, SpCas9-VRQR, SpCas9-VRER, SaCas9-KKH, SpCas9-NG, SpCas9-NRRH, SpCas9-NRTH, SpCas9-NRCH, iSpyMac, St1Cas9 LMD9-LMG18311, St1Cas9 LMD9-CNRZ1066, St1Cas9-KQKL, a variant, a fragment, a mutant, or a derivative thereof. In some embodiments, the Cas proteins or the mutant Cas proteins provided herein are a Type V Cas protein. In some embodiments, the Type V Cas protein comprises: a Cas12a, a Cas12b, a Cas12c, a Cas12d, a Cas12e, a Cas14, a Cas12g, a Cas12h, a Cas12i, a Cas12j, a Cas12k, a variant, a fragment, a mutant, or a derivative thereof. In some embodiments, the Cas protein is a chimeric Cas protein comprising domains from different Cas proteins provided herein.

In some embodiments, the Cas protein is derived from a bacterium. In some embodiments the bacterium is of the genus Streptococcus, Francisella, Lactococcus, Lactobacillus, Pseudomonas, Clostridium, Streptomyces, Actinoplanes, Synechococcus, Corynebacterium, Haloferax, Haloarcula, Methanococcus, Neisseria, Campylobacter or Staphylococcus.

In some embodiments, the Cas protein is derived from a bacterium, wherein the bacterium is Streptococcus pyogenes (e.g., SpCas9). In some embodiments, the Cas protein is an Streptococcus pyogenes Cas9 (SpCas9), wherein the Streptococcus pyogenes Cas9 comprises a sequence that is at least 90% identical to SEQ ID NO: 69. In some embodiments, the Cas protein is an Streptococcus pyogenes Cas9, wherein the an Streptococcus pyogenes Cas9 comprises a sequence that is at least 95% identical to SEQ ID NO: 69. In some embodiments, the Cas protein is an Streptococcus pyogenes Cas9, wherein the an Streptococcus pyogenes Cas9 comprises a sequence that is at least 99% identical to SEQ ID NO: 69. In some embodiments, the Cas protein is an Streptococcus pyogenes Cas9, wherein the an Streptococcus pyogenes Cas9 comprises a sequence that is identical to SEQ ID NO: 69. In some embodiments, the Streptococcus pyogenes Cas9 comprises a mutation. In some embodiments, the Streptococcus pyogenes Cas9 comprises a mutation, wherein the mutation comprises one or more amino acid substitutions.

In some embodiments, the nuclease or the nickase region of the protein construct binds to a guide polynucleotide provided herein. In some embodiments, the nuclease or the nickase region of the protein construct binds to the protein binding region of the guide polynucleotide provided herein. In some embodiments, the nuclease or the nickase region of the protein construct binds to a target nucleic acid to form a complex. In some embodiments, the complex further comprises a portion of the guide polynucleotide sequence. For example, the protein binding region and the targeting region of the guide polynucleotide form a complex between the nuclease or the nickase region of an engineered protein provided herein and the target nucleic acid via the targeting sequence.

Binding of the nuclease, the nickase, or the fragments thereof is mediated by full or partial complementarity of the targeting region of the guide polynucleotide provided herein. In some embodiments, the engineered protein construct (e.g., the nickase or the nuclease) binds to a guide polynucleotide provided herein, wherein the guide polynucleotide binds to a PAM sequence or binds adjacent to a PAM sequence. Enzymes and proteins from different bacterial species can recognize different sequence motifs or PAMs. In some embodiments, the engineered protein construct binds to the guide polynucleotide that binds to the target nucleic acid within 5 nucleobases of a PAM sequence, 10 nucleobases of a PAM sequence, 15 nucleobases of a PAM sequence, or 20 nucleobases of a PAM sequence.

Upon binding to a target nucleic acid via the guide sequence, the nucleases and nickases provided herein specifically cut the target nucleic acid at the distal end or the proximal end of the PAM. In some embodiments, the nuclease or nickase region of the protein construct generates a single-stranded break in the target nucleic acid. In some embodiments, the nickase region generates a double-stranded break in the target nucleic acid. In some embodiments, the nucleases and the nickases provided herein produce blunt ends when cutting the target nucleic acid. In some embodiments, the nucleases provided herein produce staggered ends when cutting the target nucleic acid that enables higher integration rates of synthesized DNA, improving gene editing efficiency. In some embodiments, the nucleases and the nickases provided herein cut the non-target strand (NTS). In some embodiments, the nucleases and the nickases provided herein cut the complementary strand.

Provided herein are systems, compositions, and engineered proteins that comprises a DNA ligase or a functional fragment thereof. In some embodiments, the engineered proteins comprise a DNA ligase or a DNA ligase region. In some embodiments, the DNA ligase region is operably linked to the nuclease or the nickase region or the nickase region provided herein. In some embodiments, the DNA ligase is an ATP-dependent ligase. In some embodiments, the DNA ligase is a bacterial, eukaryotic, insect or plant DNA ligase. In some embodiments, the DNA ligase is selected from the group consisting of E. coli DNA ligase, Taq DNA ligase, T4 DNA ligase, human DNA ligase I, human DNA ligase III, human DNA ligase IV. In some embodiments, the DNA ligase is T4 DNA ligase. Exemplary DNA ligase sequences are listed in Table 1.

TABLE 1
DNA Ligase Sequences.

| SEQ ID | | |
|---|---|---|
| NO: | Name/Identifier | Sequence |
| 58 | Chlorella virus PBCV-1 | MAITKPLLAATLENIEDVQFPCLATPKIDGIRSVKQTQMLSRTF |
| | DNA ligase v1 | KPIRNSVMNRLLTELLPEGSDGEISIEGATFQDTTSAVMTGHKM |
| | | YNAKFSYYWFDYVIDDPLKKYIDRVEDMKNYITVHPHILEHAQV |
| | | KIIPLIPVEINNITELLQYERDVLSKGFEGVMIRKPDGKYKFGR |
| | | STLKEGILLKMKQFKDAEATIISMTALFKNTNTKTKDNFGYSKR |
| | | STHKSGKVEEDVMGSIEVDYDGVVFSIGTGFDADQRRDFWQNKE |
| | | SYIGKMVKFKYFEMGSKDCPRFPVFIGIRHEEDR |
| 59 | Chlorella virus PBCV-1 | MTIAKPLLAATLENLDDVKFPCLVTPKIDGIRSLKQQHMLSRTF |
| | DNA ligase v2 | KPIRNSVMNKLLSELLPEGADGEICIEDSTFQATTSAVMTGHKV |
| | | YDEKFSYYWFDYVVDDPLKSYTDRVNDMKKYVDDHPHILEHEQV |
| | | KIIPLIPVEINNIDELSQYERDVLAKGFEGVMIRRPDGKYKFGR |
| | | STLKEGILLKMKQFKDAEATIISMSPRLKNTNAKSKDNLGYSKR |
| | | STHKSGKVEEETMGSIEVDYDGVVFSIGTGEDDEQRKHFWENKD |
| | | SYIGKLLKFKYFEMGSKDAPRFPVFIGIRHEEDC |
| 60 | T3 DNA ligase | MNIENTNPFKAVSFVESAVKKALETSGYLIADCKYDGVRGNIVV |
| | | DNVAEAAWLSRVSKFIPALEHINGEDKRWQQLLNDDRCIFPDGF |
| | | MLDGELMVKGVDENTGSGLLRTKWVKRDNMGFHLTNVPTKLTPK |
| | | GREVIDGKFEFHLDPKRLSVRLYAVMPIHIAESGEDYDVQNLLM |
| | | PYHVEAMRSLLVEYFPEIEWLIAETYEVYDMDSLTELYEEKRAE |
| | | GHEGLIVKDPQGIYKRGKKSGWWKLKPECEADGIIQGVNWGTEG |
| | | LANEGKVIGFSVLLETGRLVDANNISRALMDEFTSNVKAHGEDE |
| | | YNGWACQVNYMEATPDGSLRHPSFEKFRGTEDNPQEKM |
| 61 | T4 DNA ligase | MILKILNEIASIGSTKQKQAILEKNKDNELLKRVYRLTYSRGLQ |
| | | YYIKKWPKPGIATQSFGMLTLTDMLDFIEFTLATRKLTGNAAIE |
| | | ELTGYITDGKKDDVEVLRRVMMRDLECGASVSIANKVWPGLIPE |
| | | QPQMLASSYDEKGINKNIKFPAFAQLKADGARCFAEVRGDELDD |
| | | VRLLSRAGNEYLGLDLLKEELIKMTAEARQIHPEGVLIDGELVY |
| | | HEQVKKEPEGLDELFDAYPENSKAKEFAEVAESRTASNGIANKS |
| | | LKGTISEKEAQCMKFQVWDYVPLVEIYSLPAFRLKYDVRESKLE |
| | | QMTSGYDKVILIENQVVNNLDEAKVIYKKYIDQGLEGIILKNID |
| | | GLWENARSKNLYKFKEVIDVDLKIVGIYPHRKDPTKAGGFILES |
| | | ECGKIKVNAGSGLKDKAGVKSHELDRTRIMENQNYYIGKILECE |
| | | CNGWLKSDGRTDYVKLFLPIAIRLREDKTKANTFEDVFGDFHEV |
| | | TGL |
| 62 | T7 DNA ligase | MMNIKTNPFKAVSFVESAIKKALDNAGYLIAEIKYDGVRGNICV |
| | | DNTANSYWLSRVSKTIPALEHLNGEDVRWKRLLNDDRCFYKDGE |
| | | MLDGELMVKGVDENTGSGLLRTKWTDTKNQEFHEELFVEPIRKK |
| | | DKVPFKLHTGHLHIKLYAILPLHIVESGEDCDVMTLLMQEHVKN |
| | | MLPLLQEYFPEIEWQAAESYEVYDMVELQQLYEQKRAEGHEGLI |
| | | VKDPMCIYKRGKKSGWWKMKPENEADGIIQGLVWGTKGLANEGK |
| | | VIGFEVLLESGRLVNATNISRALMDEFTETVKEATLSQWGFFSP |
| | | YGIGDNDACTINPYDGWACQISYMEETPDGSLRHPSFVMERGTE |
| | | DNPQEKM |
| 63 | E. coli DNA ligase | MESIEQQLTELRTTLRHHEYLYHVMDAPEIPDAEYDRLMRELRE |
| | | LETKHPELITPDSPTQRVGAAPLAAFSQIRHEVPMLSLDNVEDE |
| | | ESFLAFNKRVQDRLKNNEKVTWCCELKLDGLAVSILYENGVLVS |
| | | AATRGDGTTGEDITSNVRTIRAIPLKLHGENIPARLEVRGEVEL |
| | | PQAGFEKINEDARRTGGKVFANPRNAAAGSLRQLDPRITAKRPL |
| | | TFFCYGVGVLEGGELPDTHLGRLLQFKKWGLPVSDRVTLCESAE |
| | | EVLAFYHKVEEDRPTLGFDIDGVVIKVNSLAQQEQLGFVARAPR |
| | | WAVAFKEPAQEQMTFVRDVEFQVGRTGAITPVARLEPVHVAGVL |
| | | VSNATLHNADEIERLGLRIGDKVVIRRAGDVIPQVVNVVLSERP |
| | | EDTREVVFPTHCPVCGSDVERVEGEAVARCTGGLICGAQRKESL |
| | | KHFVSRRAMDVDGMGDKIIDQLVEKEYVHTPADLEKLTAGKLTG |
| | | LERMGPKSAQNVVNALEKAKETTFARFLYALGIREVGEATAAGL |
| | | AAYFGTLEALEAASIEELQKVPDVGIVVASHVHNFFAEESNRNV |
| | | ISELLAEGVHWPAPIVINAEEIDSPFAGKTVVLTGSLSQMSRDD |
| | | AKARLVELGAKVAGSVSKKTDLVIAGEAAGSKLAKAQELGIEVI |
| | | DEAEMLRLLGS |
| 64 | Haemophilus | MKFYRTLLLFFASSFAFANSDLMLLHTYNNQPIEGWVMSEKLDG |
| | Influenzae DNA Ligase | VRGYWNGKQLLTRQGQRLSPPAYFIKDFPPFAIDGELESERNHE |
| | | EEISTITKSFKGDGWEKLKLYVEDVPDAEGNLFERLAKLKAHLL |
| | | EHPTTYIEIIEQIPVKDKTHLYQFLAQVENLQGEGVVVRNPNAP |
| | | YERKRSSQILKLKTARGEECTVIAHHKGKGQFENVMGALTCKNH |
| | | RGEFKIGSGFNLNERENPPPIGSVITYKYRGITNSGKPRFATYW |
| | | REKK |
| 65 | Human DNA Ligase IV | MAASQTSQTVASHVPFADLCSTLERIQKSKGRAEKIRHFREFLD |
| | | SWRKFHDALHKNHKDVTDSFYPAMRLILPQLERERMAYGIKETM |
| | | LAKLYIELLNLPRDGKDALKLLNYRTPTGTHGDAGDEAMIAYFV |
| | | LKPRCLQKGSLTIQQVNDLLDSIASNNSAKRKDLIKKSLLQLIT |
| | | QSSALEQKWLIRMIIKDLKLGVSQQTIESVFHNDAAELHNVTTD |
| | | LEKVCRQLHDPSVGLSDISITLFSAFKPMLAAIADIEHIEKDMK |
| | | HQSFYIETKLDGERMQMHKDGDVYKYFSRNGYNYTDQFGASPTE |
| | | GSLTPFIHNAFKADIQICILDGEMMAYNPNTQTFMQKGTKEDIK |
| | | RMVEDSDLQTCYCVFDVLMVNNKKLGHETLRKRYEILSSIFTPI |
| | | PGRIEIVQKTQAHTKNEVIDALNEAIDKREEGIMVKQPLSIYKP |
| | | DKRGEGWLKIKPEYVSGLMDELDILIVGGYWGKGSRGGMMSHFL |
| | | CAVAEKPPPGEKPSVFHTLSRVGSGCTMKELYDLGLKLAKYWKP |
| | | FHRKAPPSSILCGTEKPEVYIEPCNSVIVQIKAAEIVPSDMYKT |
| | | GCTLRFPRIEKIRDDKEWHECMTLDDLEQLRGKASGKLASKHLY |
| | | IGGDDEPQEKKRKAAPKMKKVIGIIEHLKAPNLINVNKISNIFE |
| | | DVEFCVMSGTDSQPKPDLENRIAEFGGYIVQNPGPDTYCVIAGS |
| | | ENIRVKNIILSNKHDVVKPAWLLECEKTKSFVPWQPREMIHMCP |
| | | STKEHFAREYDCYGDSYFIDTDLNQLKEVFSGIKNSNEQTPEEM |
| | | ASLIADLEYRYSWDCSPLSMFRRHTVYLDSYAVINDLSTKNEGT |
| | | RLAIKALELRFHGAKVVSCLAEGVSHVIIGEDHSRVADFKAFRR |
| | | TFKRKFKILKESWVTDSIDKCELQEENQYLI |
| 66 | Human DNA Ligase I | MQRSIMSFFHPKKEGKAKKPEKEASNSSRETEPPPKAALKEWNG |
| | | VVSESDSPVKRPGRKAARVLGSEGEEEDEALSPAKGQKPALDCS |
| | | QVSPPRPATSPENNASLSDTSPMDSSPSGIPKRRTARKQLPKRT |
| | | IQEVLEEQSEDEDREAKRKKEEEEEETPKESLTEAEVATEKEGE |
| | | DGDQPTTPPKPLKTSKAETPTESVSEPEVATKQELQEEEEQTKP |
| | | PRRAPKTLSSFFTPRKPAVKKEVKEEEPGAPGKEGAAEGPLDPS |
| | | GYNPAKNNYHPVEDACWKPGQKVPYLAVARTFEKIEEVSARLRM |
| | | VETLSNLLRSVVALSPPDLLPVLYLSLNHLGPPQQGLELGVGDG |
| | | VLLKAVAQATGRQLESVRAEAAEKGDVGLVAENSRSTQRLMLPP |
| | | PPLTASGVFSKFRDIARLTGSASTAKKIDIIKGLFVACRHSEAR |
| | | FIARSLSGRLRLGLAEQSVLAALSQAVSLTPPGQEFPPAMVDAG |
| | | KGKTAEARKTWLEEQGMILKQTFCEVPDLDRIIPVLLEHGLERL |
| | | PEHCKLSPGIPLKPMLAHPTRGISEVLKRFEEAAFTCEYKYDGQ |
| | | RAQIHALEGGEVKIFSRNQEDNTGKYPDIISRIPKIKLPSVTSF |
| | | ILDTEAVAWDREKKQIQPFQVLTTRKRKEVDASEIQVQVCLYAF |
| | | DLIYLNGESLVREPLSRRRQLLRENFVETEGEFVFATSLDTKDI |
| | | EQIAEFLEQSVKDSCEGLMVKTLDVDATYEIAKRSHNWLKLKKD |
| | | YLDGVGDTLDLVVIGAYLGRGKRAGRYGGFLLASYDEDSEELQA |
| | | ICKLGTGESDEELEEHHQSLKALVLPSPRPYVRIDGAVIPDHWL |
| | | DPSAVWEVKCADLSLSPIYPAARGLVDSDKGISLRFPRFIRVRE |
| | | DKQPEQATTSAQVACLYRKQSQIQNQQGEDSGSDPEDTY |

In some embodiments, the DNA ligase, the DNA ligase region, or the DNA ligase fragment binds to the ligation splint 1 region of the guide polynucleotide. In some embodiments, the DNA ligase does not bind to the target nucleic acid sequence. In some embodiments, the DNA ligase attaches the 5′ end of the donor nucleic acid to the 3′ end of the leading strand. The donor nucleic acid can replace at least a portion of a target sequence in a double-stranded target nucleic acid, thereby editing the double-stranded target nucleic acid.

In some embodiments, an engineered protein provided herein further comprises one or more additional protein regions or protein constructs, wherein the one or more additional protein region or protein construct comprises: a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof. In some embodiments, the compositions and systems provided herein comprise an additional engineered protein. In some embodiments, the additional engineered protein comprises: a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof.

In some embodiments, an engineered protein provided herein comprises a protein construct that modulates transcription. In some embodiments, an engineered protein provided herein comprises a transcriptional repressor protein construct. In some embodiments, an engineered protein provided herein comprises a zinc finger protein construct. In some embodiments, the zinc finger protein construct comprises a Krüppel-associated box (KRAB) protein or a functional fragment thereof. In some embodiments, the engineered protein further comprises a KRAB domain that binds to a transcriptional corepressor protein. In some embodiments, an engineered protein provided herein comprises a SUMO protein construct.

In some embodiments, an engineered protein provided herein comprises a transcriptional activator protein construct. A transcriptional activator protein construct recruits transcription factors from a host cell to the target nucleic acid for regulation of target nucleic acid expression. Exemplary transcriptional activators include VP64, VP16, VP160, VP48, VP96, p65, Rta, VPR, hsf1, and p300. In some embodiments, an engineered protein provided herein comprises one or more VP16 protein construct. In some embodiments, an engineered protein provided herein comprises one or more VP64 protein construct. In some embodiments, an engineered protein provided herein comprises one or more VPR protein construct. In some embodiments, an engineered protein provided herein comprises one or more SunTag protein construct. In some embodiments, an engineered protein provided herein comprises VP64, p65, and HSF1 (SunTag-p65-heat-shock factor 1 or SPH). In some embodiments, an engineered protein provided herein comprises a CREB-binding protein (CPB). CBP can be used to recruit transcriptional machinery and function as a histone acetyltransferase (HAT) that alters chromatin structure.

In some embodiments, an engineered protein provided herein further comprises an aptamer. In some embodiments, the aptamers are bind to one or more MS2 proteins.

In some embodiments, an engineered protein provided herein comprises a self-cleaving protein sequence. Non-limiting examples of self-cleaving protein sequences include: E2A, P2A and T2A. In some embodiments, an engineered protein provided herein further comprises an antibiotic resistance protein construct or an antibiotic resistance selectable marker. Non-limiting examples of antibiotic resistance proteins and selectable markers include: aminoglycoside acetyltransferase, rifampin ADP-ribosyltransferase, dihydrofolate reductase, multidrug and toxic compound extrusion transporters, antibiotic resistance ATP-binding cassette family F (ARE ABC-F) proteins, β-lactamase, blasticidin-S deaminase, penicillin-binding proteins (PBPs), and puromycin-N-acetyltransferase. In some embodiments, the ARE ABC-F proteins are MsrE, Erm, Vga, Lsa, Sal, or OptrA.

In some embodiments, an engineered protein provided herein comprises a base editor. In some embodiments, the base editor is selected from the group consisting of: an adenine base editor, an adenosine base editor, a cytidine deaminase, a cytosine to guanine base editor, a deaminase dimer. In some embodiments, the cytidine deaminase is an activation-induced deaminase (AID), an APOBEC deaminase, APOBEC3G, APOBEC1, cytidine deaminase 1 (CDA1), a functional fragment, or a derivative thereof. In some embodiments, the adenosine base editor is ecTadA, saTadA, a functional fragment, or a derivative thereof. In some embodiments, the engineered proteins provided herein further comprise a uracil-DNA glycosylase, a uracil-DNA glycosylase inhibitor, a functional fragment, or a derivative thereof.

In some embodiments, a system, a composition, or an engineered protein provided herein further comprises a nuclear localization sequence (NLS). An NLS targets a protein to the nucleus of a cell, localizing an engineered protein provided herein in close proximity to a target nucleic acid within the nucleus of a cell. In some embodiments, a system, a composition, or an engineered protein comprises more than one nuclear localization sequence (NLS). In some embodiments, the NLS is from a Simian Vacuolating Virus 40 (SV40). In some embodiments, the NLS comprises a monopartite SV40 NLS. In some embodiments, the NLS comprises a bipartite SV40 NLS. In some embodiments, the NLS comprises PKKKRKV (SEQ ID NO: 74) or KRTADGSEFEPKKKRKV (SEQ ID NO: 75). In some embodiments, the NLS comprises a nucleoplasmin sequence. In some embodiments, the nucleoplasmin sequence comprises: KRPAATKKAGQAKKKK (SEQ ID NO: 76).

In some embodiments, a system, a composition, or an engineered protein provided herein further comprises a linker. A linker is a molecular entity that can directly or indirectly connect at two parts of a composition. For example, the linker can connect the first protein construct to the second protein construct and the second protein construct to a third protein construct, and so on. Linkers can be configured according to a specific need. For example, the linkers can be configure for improved stability or achieve the optimal length between two amino acid sequences. In some embodiments, linkers can be configured to allow multimerization of the nuclease or the nickase with a DNA ligase provided herein, for example, from a monomer to a di-, tri-, tetra-, penta-, or higher multimeric complex) while retaining biological activity. In some embodiments, the biological activity comprises cleavage of a target nucleic acid or a set of target nucleic acids. In some embodiments, a linker can be selected from (GS)ₙ (SEQ ID NO: 229) or (GGS)ₙ (SEQ ID NO: 230), where n is 1, 2, 3, 4, or 5. Other exemplary linkers are provided in Table 2.

TABLE 2
Linker Sequences.

| | Sequence (where |
|---|---|
| SEQ ID NO: | ₙ = a number between 1 and 1000) |
| SEQ ID NO: 77 | (GGS)ₙ |
| SEQ ID NO: 78 | (GGGGS)ₙ |
| SEQ ID NO: 79 | (EAAAK)ₙ |
| SEQ ID NO: 80 | MSRPDPA |
| SEQ ID NO: 81 | MKIIEQLPSA |
| SEQ ID NO: 82 | VRHKLKRVGS |
| SEQ ID NO: 83 | SIVAQLSRPDPA |
| SEQ ID NO: 84 | GHGTGSTGSGSS |
| SEQ ID NO: 85 | GSAGSAAGSGEF |
| SEQ ID NO: 86 | VPFLLEPDNINGKTC |
| SEQ ID NO: 87 | SGSETPGTSESATPES |
| SEQ ID NO: 88 | SGGSSGGSSGSETPGTSESATPESSGGSSGGSS |

In some embodiments, linkers are configured to facilitate expression and purification of the nuclease or the nickase provided herein. In some embodiments, an engineered protein provided herein comprises a cleavable linker or a non-cleavable linker between the different protein constructs or domains of the engineered protein. For example, a linker can be a polypeptide linker, such as a linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids long. Generally, the number of additional elements in the engineered protein will depend on a number of factors, including, for example, size or molecule weight of the protein for delivery to a cell, or translocation of the protein desired in a cell type of interest.

Provided herein are engineered protein constructs and polynucleotides encoding for engineered proteins, wherein the engineered protein constructs comprise an engineered nickase and a DNA ligase or a functional fragment thereof. In some embodiments, In some embodiments, the engineered protein constructs further comprise a linker between the nickase region or the nuclease region; and the DNA ligase or the functional fragment thereof. In some embodiments, the linker comprises an amino acid sequence that is at least 95% identical to any one of SEQ ID NOS: 77-88. In some embodiments, the linker comprises an amino acid sequence that is at least 99% identical to any one of SEQ ID NOS: 77-88. In some embodiments, the linker comprises any one of SEQ ID NOS: 77-88. In some embodiments,

### Combination Compositions

Provided herein are compositions comprising: (a) a donor nucleic acid; (b) a polynucleotide encoding a nickase or a variant thereof; (c) a polynucleotide encoding a protein construct comprising a nickase or a variant thereof and a DNA ligase or a functional fragment thereof; and (d) a guide polynucleotide described herein. In some embodiments, the nickase is an engineered Cas protein or a functional variant thereof. In some embodiments, the engineered Cas protein or a functional variant thereof comprises at least one amino acid substitution at position 840 corresponding to SEQ ID NO: 69. In some embodiments, the engineered Cas protein or a functional variant thereof further comprises at least one amino acid substitution at position 221, 394, or a combination thereof, corresponding to SEQ ID NO: 69. In some embodiments, the engineered Cas protein or a functional variant thereof comprises amino acid substitutions of R221K, N394K, H840A, or any combination thereof. In some embodiments, the engineered Cas protein or a functional variant thereof comprises amino acid substitutions of H840A. In some embodiments, the engineered Cas protein or a functional variant thereof comprises amino acid substitutions of R221K. In some embodiments, the engineered Cas protein or a functional variant thereof comprises amino acid substitutions of N394K. In some embodiments, the engineered Cas protein or a functional variant thereof comprises a sequence 97%, 98%, 99%, or 100% identical to SEQ ID NO: 70 or SEQ ID NO: 71. In some embodiments, the engineered Cas protein or a functional variant thereof comprises a sequence of SEQ ID NO: 70 or SEQ ID NO: 71. In some embodiments, the DNA ligase or a functional fragment comprises an E. coli DNA ligase, a Taq DNA ligase, a T3 DNA ligase, a T4 DNA ligase, a T7 DNA ligase, a Chlorella virus DNA ligase, a human DNA ligase I, a human DNA ligase II, a human DNA ligase III, a human DNA ligase IV, a human ligase V, a variant, or a combination thereof. In some embodiments, the DNA ligase or a functional fragment thereof comprises Chlorella virus DNA ligase, a T4 DNA ligase, or a human DNA ligase IV. In some embodiments, the DNA ligase or a functional fragment thereof comprises a sequence at least 97%, at least 98%, at least 99%, or 100% identical to a sequence of SEQ ID NOS: 58 to 66. In some embodiments, the DNA ligase or a functional fragment thereof comprises a sequence of SEQ ID NOS: 58 to 66. In some embodiments, the DNA ligase or a functional fragment thereof comprises a Chlorella virus DNA ligase comprising a sequence at least 80%, at least 85%, at least 90%, at least 95%, at least 99%, or 100% identical to a sequence of SEQ ID NO: 58. In some embodiments, the DNA ligase or a functional fragment thereof comprises a Chlorella virus DNA ligase comprising a sequence of SEQ ID NO: 58.

Provided herein are engineered fusion proteins comprising (a) a DNA ligase or a functional fragment thereof; and (b) an engineered nickase that comprises three amino acid substitutions at positions corresponding to amino acid positions 221, 394, and 840 of a nuclease comprising a sequence of SEQ ID NO: 69. In some embodiments, the substitutions result in enhanced nickase activity as compared to an otherwise equivalent engineered nickase. In some embodiments, the engineered nickase comprises amino acid substitutions of R221K, N394K, and H840A. In some embodiments, the engineered nickase or a functional variant thereof comprises a sequence 97%, 98%, 99%, or 100% identical SEQ ID NO: 71. In some embodiments, the engineered nickase or a functional variant thereof comprises a sequence of SEQ ID NO: 71. In some embodiments, the DNA ligase or a functional fragment comprises an E. coli DNA ligase, a Taq DNA ligase, a T3 DNA ligase, a T4 DNA ligase, a T7 DNA ligase, a Chlorella virus DNA ligase, a human DNA ligase I, a human DNA ligase II, a human DNA ligase III, a human DNA ligase IV, a human ligase V, a variant, or a combination thereof. In some embodiments, the DNA ligase or a functional fragment thereof comprises Chlorella virus DNA ligase, a T4 DNA ligase, or a human DNA ligase IV. In some embodiments, the DNA ligase or a functional fragment thereof comprises a sequence 97%, 98%, 99%, or 100% identical to SEQ ID NOS: 58 to 66. In some embodiments, the DNA ligase or a functional fragment thereof comprises a sequence of SEQ ID NOS: 58 to 66. In some embodiments, the DNA ligase or a functional fragment thereof comprises a Chlorella virus DNA ligase comprising a sequence at least 80%, at least 85%, at least 90%, at least 95%, at least 99%, or 100% identical to a sequence of SEQ ID NO: 58. In some embodiments, the DNA ligase or a functional fragment thereof comprises a Chlorella virus DNA ligase comprising a sequence of SEQ ID NO: 58. In some embodiments, the DNA ligase or a functional fragment thereof comprises a Chlorella virus DNA ligase comprising a sequence at least 80%, at least 85%, at least 90%, at least 95%, at least 99%, or 100% identical to a sequence of SEQ ID NO: 58. In some embodiments, the DNA ligase or a functional fragment thereof comprises a Chlorella virus DNA ligase comprising a sequence of SEQ ID NO: 58. In some embodiments, the engineered fusion protein further comprises a linker, a nuclear localization sequence (NLS), or an additional protein construct. In some embodiments, the additional protein construct comprises a cell-targeting moiety, a receptor-targeting moiety, a regulatory element, a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof.

Provided herein are engineered fusion protein comprising an amino acid sequence at least 90% identical to any one of SEQ ID NOS: 89-101. Provided herein are engineered fusion protein comprising an amino acid sequence at least 95% identical to any one of SEQ ID NOS: 89-101. Provided herein are engineered fusion protein comprising an amino acid sequence at least 96% identical to any one of SEQ ID NOS: 89-101. Provided herein are engineered fusion protein comprising an amino acid sequence at least 97% identical to any one of SEQ ID NOS: 89-101. Provided herein are engineered fusion protein comprising an amino acid sequence at least 98% identical to any one of SEQ ID NOS: 89-101. Provided herein are engineered fusion protein comprising an amino acid sequence at least 99% identical to any one of SEQ ID NOS: 89-101. Provided herein are engineered fusion protein comprising an amino acid sequence at least 95% identical to any one of SEQ ID NOS: 89-101, wherein the engineered fusion proteins comprise at least one, at least two, at least three, at least four, at least five, at least six, or at least seven amino acid substitutions in the DNA ligase region. Provided herein are engineered fusion protein comprising an amino acid sequence at least 95% identical to any one of SEQ ID NOS: 89-101, wherein the engineered fusion proteins comprise at least one, at least two, at least three, at least four, at least five, at least six, or at least seven amino acid substitutions in the engineered nickase region. Provided herein are engineered fusion protein comprising an amino acid sequence of SEQ ID NOS: 89-101.

Provided herein are nucleic acids and polynucleotides encoding any one of the engineered fusion proteins provided herein. In some embodiments, the polynucleotide encoding the engineered fusion protein comprises DNA. In some embodiments, the polynucleotide encoding the engineered fusion protein comprises RNA. Provided herein are vectors comprising any nucleic acid or polynucleotide provided herein or a plurality of nucleic acids or polynucleotides as provided herein. In some embodiments, the nucleic acids, vectors, or viral vectors provided herein further comprise a guide polynucleotide provided herein. In some embodiments, the nucleic acids, vectors, or viral vectors provided herein further comprise a DNA encoding for a guide polynucleotide provided herein.

### Target Nucleic Acids

A guide polynucleotide provided herein can comprise a degree of complementarity to a target polynucleotide sequence of interest or a strand thereof. A targeting region of a guide polynucleotide provided herein can comprise at least partial sequence complementarity to a target polynucleotide. The targeting sequence may have a degree of sequence complementarity to the target nucleic acid that is sufficient for the guide polynucleotide to hybridize with the target polynucleotide. In some cases, the targeting sequence comprises 95%, 96%, 97%, 98%, 99%, or 100% sequence complementarity to the target polynucleotide. Sequence complementarity can be determined by using alignment methods known in the art, for instance alignment of the sequences can be conducted using publicly available software such as BLAST, Align, ClustalW2. In some embodiments, a target nucleic acid provided herein comprises a gene or a polynucleotide comprising DNA. In some embodiments, the gene or the polynucleotide comprises a mammalian gene or polynucleotide. In some embodiments, the gene or the polynucleotide comprises a human gene or polynucleotide. In some embodiments, a target nucleic acid provided herein comprises a DNA. In some embodiments, a target nucleic acid provided herein comprises a single-stranded DNA. In some embodiments, a target nucleic acid provided herein comprises double-stranded DNA In some embodiments, a target nucleic acid provided herein comprises RNA. In some embodiments, a target nucleic acid provided herein comprises a single-stranded RNA. In some embodiments, a target nucleic acid provided herein comprises double-stranded RNA.

In some embodiments, the target nucleic acid or a complementary strand provided herein comprises a protospacer-adjacent motif (“PAM”), wherein the PAM is a short T-rich sequence. In some embodiments, cleavage of a target nucleic acid by the engineered protein provided herein occurs downstream or 3′ from the PAM sequence. In some embodiments, cleavage of a target nucleic acid occurs upstream or 5′ from the PAM sequence. In some embodiments, the PAM is an NGG PAM sequence. In some embodiments, the PAM comprises NGAN, NGNG, NGAG, NGCG, wherein N is A, G, C or T. In some embodiments, the PAM is a T-rich PAM. In some embodiments, the PAM has the nucleotide sequence (T)XN, wherein the X is the number of thymines (e.g, 1-10), and Nis A, G, C or T. In certain embodiments, X is equal to 2, and thus, the PAM is TTN. In some embodiments, X is 3, and thus, the PAM is TTTN. In some embodiments, the nuclease or the nickase provided herein recognizes the sequence motif TTTN and directs cleavage of a target nucleic acid sequence 1-24 bp (e.g., 1, 2, 3, 4, 5, 6, 7, 8, 9, 10, 11, 12, 13, 14, 15, 16, 17, 18, 19, 20, 21, 22, 23, or 24) downstream from that sequence.

Without limitation, the target nucleic acid sequence can be from any cell or organism. Determining the appropriate sequence for the guide polynucleotide to bind to a target nucleic acid provided herein will depend on the sequence of the desired target and structure of the nuclease or the nickase provided herein. Methods of designing the targeting region of a guide polynucleotide for gene editing are known in the art and include, for example, using software and databases such as Breaking-Cas, Cas-OFFinder, CRISPR-DT, CHOPCHOP, CCTOP, CRISPick, or CRISPOR.

In some embodiments, the target nucleic acid comprises a double-stranded DNA (dsDNA). In the case of dsDNA, the polynucleotide and the nickase region of the engineered protein provided herein binds to the target nucleic acid and generates a single-strand break in the dsDNA. The cleavage can occur on the bottom strand or the top strand of the double stranded DNA. Cleavage of the dsDNA generates a DNA replication fork and a leading strand and a complementary strand (also called a lagging strand) form. The guide polynucleotide binds to both strands of the target DNA via different regions of the guide polynucleotide. For example, the ligation splint 1 region binds to the leading strand of the DNA following cleavage of the dsDNA by a nickase or a nickase region of an engineered polynucleotide. The targeting region of the guide polynucleotide comprises RNA and binds to the complementary strand that comprises a PAM sequence. The ligation splint 2 region mediates hybridization with the ligation donor nucleic acid. Once the ligation splint 1 region is bound to the leading strand and the donor nucleic acid hybridizes to the ligation splint 2 region, the DNA ligase of the engineered protein catalyzes the formation of a phosphodiester bond between the 3′ hydroxyl ends of the leading strand and the 5′ phosphate end of the donor nucleic acid. The DNA ligase does not bind to the complementary strand comprising the PAM sequence on the target nucleic acid. Once, the DNA ligase has completed the ligation, the guide polynucleotide dissociates from the complementary strand of the nicked target nucleic acid and the donor nucleic acid is incorporated into the target nucleic acid. In some cases, a DNA bulge forms where the DNA edit is included in the ligated DNA sequence as the new sequence differs from the complementary target DNA strand.

Additional DNA mismatch repair proteins recognize the bulge and can promote the correction of an aberrant sequence in the target nucleic acid (relative to a wild-type reference sequence) and modify the target nucleic acid. In some embodiments, the target nucleic acid may be repaired by DNA mismatch repair (MMR) proteins, homology directed repair (HDR) proteins or non-homologous end joining (NHEJ) repair proteins. In some embodiments, the MMR proteins, the HDR proteins, and/or the NHEJ proteins are endogenous to a cell. In some embodiments, the MMR proteins, the HDR proteins, and/or the NHEJ proteins are introduced to a cell or a cell-free system. In some cases, when the nickase cleaves the bottom strand of DNA, for example, the complementary strand, away from the edit, the donor nucleic acid or the LS2 region can integrate into the genome.

### (2) Delivery Vehicles and Vectors

Provided herein are compositions comprising a guide polynucleotide provided herein and a delivery vehicle. Provided herein are compositions comprising an engineered protein provided herein and a delivery vehicle. Provided herein are systems provided herein and one or more delivery vehicles. The compositions and cells provided herein can be delivered to a target cell, tissue, organ, or subject by any suitable means.

The engineered proteins, guide polynucleotides, and any polynucleotide encoding for the engineered proteins or the guide polynucleotides provided herein can be admixed with a delivery vehicle that permits delivery of the system to the target nucleic acid sequence within a cell, tissue, or subject. Polynucleotides and sets of polynucleotides that encode the engineered protein and/or the guide polynucleotide provided herein comprises DNA, RNA, or both DNA and RNA. In some embodiments, an RNA encodes for the engineered protein provided herein or the guide polynucleotides provided herein. For example, RNA delivery of proteins can improve expression of the engineered proteins in human cells. In some embodiments, the polynucleotides provided herein comprises a self-replicating RNA or a viral RNA.

In some embodiments, the delivery vehicle is a liposome. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of ML Vs results in the formation of small unilamellar vesicles (SUVs) with diameters in the range of 200 to 500 angstroms containing an aqueous solution in the core. Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles can also be used as a delivery vehicle.

In some embodiments, the delivery vehicle is a phospholipid. Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a tightly packed, ordered structure, known as the gel state, to a loosely packed, less-ordered structure, known as the fluid state. This occurs at a characteristic phase-transition temperature and results in an increase in permeability to ions, sugars and drugs.

In some embodiments, the delivery vehicle is a nanoparticle. Nanoparticle carriers that specifically target a tissue provided herein may also be used as a pharmaceutically acceptable carrier. In some embodiments, the nanoparticle is a gold nanoparticle, a platinum nanoparticle, an iron-oxide nanoparticle, a lipid nanoparticle, a selenium nanoparticle, a tumor-targeting glycol chitosan nanoparticle (CNP), a cathepsin B sensitive nanoparticle, a hyaluronic acid nanoparticle, a paramagnetic nanoparticle, or a polymeric nanoparticle. In some embodiments, the delivery vehicle is a lipid nanoparticle.

Provided herein are compositions comprising a lipid nanoparticle comprising: a system provided herein, a set of polynucleotides encoding the system provided herein, a vector provided herein, an engineered protein provided herein, a guide polynucleotide or a portion thereof, or any composition provided herein. In some embodiments, the lipid nanoparticle is a solid lipid nanoparticle (SLN) or a nanostructured lipid carrier (NLC).

The lipid nanoparticles provided herein can comprise cationic lipids, ionizable lipids, a mixture of lipids, zwitterionic lipids, and/or phospholipids. In some embodiments, the lipid nanoparticle comprises a cationic lipid selected from the group consisting of: 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), and Ethylphosphatidylcholine (ePC). In some embodiments, the lipid nanoparticle comprises an ionizable lipid selected from the group consisting of: 2S)-2,5-bis(3-aminopropylamino)-N-[2-(dioctadecylamino)acetyl]pentanamide (DOGS; Transfectam), N1-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol and N4-cholesteryl-spermine (GL67), 9Z,12Z-octadecadienoic acid, 3-[4,4-bis(octyloxy)-1-oxobutoxy]-2-[[[3-(diethylamino)propoxy]carbonyl]oxy]methyl]propyl ester (LP01), heptadecan-9-yl 8-[2-hydroxyethyl-(6-oxo-6-undecoxyhexyl)amino]octanoate (SM-102), [(4-Hydroxybutyl)azanediyl]di(hexane-6,1-diyl)bis(2-hexyldecanoate) (ALC-0315), bis(2-butyloctyl) 10-(N-(3-(dimethylamino)propyl)nonanamido)nonadecanedioate (Lipid A9), 5-(dimethylamino)-pentanoic acid, (6Z)-1,2-di-(4Z)-4-decen-1-yl-6-dodecen-1-yl ester (Lipid CL1), 7-[(2-Hydroxyethyl)[8-(nonyloxy)-8-oxooctyl]amino]heptyl 2-octyldecanoate (Lipid 5). In some embodiments, the lipid nanoparticle comprises a phosphatidylcholine. In some embodiments, the lipid nanoparticle comprises cholesterol or a cholesterol analog. In some embodiments, the lipid nanoparticle comprises a cholesterol analog selected from the group consisting of: β-sitosterol, Vitamin D3, Vitamin D2, calcipotriol, stigmasterol, betulin, lupeol, ursolic acid, oleanolic acid, stigmastanol, campesterol, fucosterol, brassicasterol, and ergosterol. In some embodiments, the lipid nanoparticle comprises polyethylene glycol (PEG). In some embodiments, the PEG is 1,2-dimyristoyl-rac-glycero-3-methoxypolyethylene glycol-2000 (PEG₂₀₀₀-DMG) or 1,2-distearoyl-rac-glycero-3-methoxypolyethylene glycol-2000 (PEG₂₀₀₀-DSG). In some embodiments, the lipid nanoparticle comprises N-acetylgalactosamine (GalNAc). In some embodiments, the GalNAc is 1,2-distearoyl-sn-glycero-3-phosphoethanolamine-N-[tris-GalNAc-GABA-(polyethylene glycol)-(Tri-GalNAc-PEG2000-DSPE).

In some embodiments, the composition comprising the lipid nanoparticle is in the form of a emulsion. In some embodiments, the composition comprising the lipid nanoparticle is in the form of a liquid. In some embodiments, the composition comprising the lipid nanoparticle is in the form of a gel. In some embodiments, the composition comprising the lipid nanoparticle is in the form of a solid. In some embodiments, the system provided herein, the set of polynucleotides encoding the system provided herein, the vector provided herein, the engineered protein provided herein, or the guide polynucleotide or a portion thereof are encapsulated by the lipid nanoparticle. In some embodiments, the system provided herein, the set of polynucleotides encoding the system provided herein, the vector provided herein, the engineered protein provided herein, or the guide polynucleotide or a portion thereof are in complex with the lipid nanoparticle.

The compositions provided herein can be delivered to a cell system using vectors, for example containing polynucleotide sequences encoding a system, a guide polynucleotide, an engineered protein, or a composition provided herein. In some embodiments, a system as described herein can be delivered absent a viral vector. Any vector systems can be used including, but not limited to, plasmid vectors, viral vectors, and oncolytic viral vectors. Furthermore, any of these vectors can comprise one or more transcription factor, transgene, or molecular tag.

In some embodiments, the vectors provided herein are viral vectors. Exemplary viral vectors include, but are not limited to, lentiviral vectors, retroviral vectors, adeno-associated viral vectors (AAV), adenoviral vectors, herpes simplex viral vectors, alphaviral vectors, flaviviral vectors, rhabdoviral vectors, measles viral vectors, Newcastle disease viral vectors, poxviral vectors, picornaviral vectors, and oncolytic viral vectors.

In some embodiments, the viral vector comprises an AAV. AAVs can have one or more of the AAV wild-type genes deleted in whole or part. For example, the rep and/or cap genes of the AAV can be deleted in whole or in part, but can still retain functional flanking ITR sequences. Functional ITR sequences are necessary for the rescue, replication, and packaging of the AAV virion. The ITRs need not be the wild-type nucleotide sequences, and may be altered, for example, by the insertion, deletion or substitution of nucleotides, so long as the sequences provide for functional rescue, replication and packaging. A recombinant AAV vector (rAAV) comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery. In some embodiments, the AAV or the rAAV provided herein comprises a serotype of AAV1, AAV2, AAV3, AAV4, AAV5, AAV6, AAV7, AAV8, AAV9, AAV10, AAV11, AAV12, AAV13, AAVrh10, or any combination thereof. In some embodiments, the delivery vehicle comprises a hybrid AAV-lipid nanoparticle delivery system.

In some embodiments, the viral vector is a lentiviral vector. In some embodiments, the lentiviral vector is selected from the group consisting of: a human immunodeficiency virus 1 (HIV-1); a human immunodeficiency virus 2 (HIV-2), a visna-maedi virus (VMV) virus; a caprine arthritis-encephalitis virus (CAEV); an equine infectious anemia virus (EIAV); a feline immunodeficiency virus (FIV); a bovine immune deficiency virus (BIV); and a simian immunodeficiency virus (SIV), fragments, derivatives, or variants thereof.

Conventional viral and non-viral based gene transfer methods can be used to introduce polynucleotides encoding for a composition, system, guide polynucleotide, or an engineered protein provided herein to cells and target tissues. Exemplary non-viral vector delivery systems can include DNA plasmids, naked nucleic acid, and nucleic acids complexed with a delivery vehicle such as a liposome or poloxamer. Viral vector delivery systems can also include DNA and RNA viruses, which have either episomal or integrated genomes after delivery to the cell.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, for example, a Sonitron 2000 system (Rich-Mar) can also be used for delivery of nucleic acids. Additional exemplary nucleic acid delivery systems include those provided by Lonza Nucleofactor technologies (Cologne, Germany), Life Technologies (Frederick, Md.), MAXCYTE, Inc. (Rockville, Md.), BTX Molecular Delivery Systems (Holliston, Mass.) and Copernicus Therapeutics Inc. Lipofection reagents are sold commercially (e.g., TRANSFECTAM® and LIPOFECTIN®).

Delivery of the compositions and systems provided herein can be to cells (ex vivo administration) or target tissues (in vivo administration). Additional methods of delivery include the use of packaging the polynucleotides to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis.

Vectors including viral and non-viral vectors containing nucleic acids encoding a nucleic editing system provided herein can also be administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. More than one route can be used to administer a particular composition.

In some embodiments, a composition, engineered protein, guide polynucleotide, or a system provided herein can be shuttled to a cellular nucleus. For example, a vector can contain a nuclear localization sequence (NLS). A vector or any composition provided herein can also be shuttled by a protein or protein complex. In some embodiments, a composition or a system provided herein can be introduced to a cell or a target tissue by a minicircle vector.

In some embodiments, a vector or a polynucleotide provided herein can be pre-complexed with an engineered protein provided herein prior to electroporation into a cell. An engineered protein that can be used for shuttling can be a nickase or a catalytically dead Cas protein. A nuclease that can be used for shuttling can be a nuclease-competent protein. In some embodiments, an engineered protein herein can be pre-mixed with a guide polynucleotide provided herein an any additional elements such as transgenes or other engineered proteins.

A cell can be transfected with a mutant or chimeric adeno-associated viral vector encoding a system or a composition provided herein. For example, an AAV vector concentration can be from about 0.5 nanograms up to 50 micrograms.

A system or a composition provided herein can also be introduced to a cell via electroporation techniques. The amount of polynucleotides that can be introduced into the cell by electroporation can be varied to optimize transfection efficiency and/or cell viability. In some embodiments, less than about 100 picograms of nucleic acid can be added to each cell sample, which can include one or more cells being electroporated. In some embodiments, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid can be added to each cell sample. For example, 1 microgram of nucleic acids, polynucleotides, vectors, or compositions provided herein can be added to each cell sample for electroporation. In some embodiments, the amount of nucleic acids required for optimal transfection efficiency and/or cell viability can be specific to the cell type. In some embodiments, the amount of nucleic acids used for each sample can directly correspond to the transfection efficiency and/or cell viability. The transfection efficiency of cells with any of the nucleic acid delivery platforms described herein, for example, nucleofection or electroporation, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Viral particles, such as AAV, can be used to deliver a viral vector comprising a gene of interest or a transgene into a cell ex vivo or in vivo. In some embodiments, a mutated or chimeric adeno-associated viral vector as disclosed herein can be measured as pfu (plaque forming units). In some embodiments, the pfu of recombinant virus or mutated or chimeric adeno-associated viral vector of the compositions and methods of the disclosure can be about 10⁸ to about 5×10¹⁰ pfu. In some embodiments, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some aspects, a mutated or chimeric adeno-associated viral vector of the disclosure can be measured as vector genomes. In some embodiments, recombinant viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some embodiments, a mutated or chimeric adeno-associated viral vector of the disclosure can be measured using multiplicity of infection (MOI). In some embodiments, MOI can refer to the ratio, or multiple of vector or viral genomes to the cells to which the nucleic can be delivered. In some embodiments, MOI can refer to the ratio, or multiple of vector or viral genomes to the cells to which the nucleic can be delivered. In some embodiments, the MOI can be 1×10⁶ GC/mL. In some embodiments, the MOI can be 1×10⁵ GC/mL to 1×10⁷ GC/mL. In some embodiments, the MOI can be 1×10⁴ GC/mL to 1×10⁸ GC/mL. In some embodiments, recombinant viruses of the disclosure are at least about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some embodiments, a mutated or chimeric adeno-associated viruses of this disclosure are from about 1×10⁸ GC/mL to about 3×10¹⁴ GC/mL MOI, or are at most about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI.

In some aspects, a non-viral vector or nucleic acid can be delivered without the use of a mutated or chimeric adeno-associated viral vector and can be measured according to the quantity of nucleic acid. Generally, any suitable amount of nucleic acid can be used with the compositions and methods of this disclosure. In some embodiments, nucleic acid can be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some embodiments, nucleic acid can be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

Proteins, vectors, plasmids, compositions, systems, engineered proteins, and guide polynucleotides provided herein can be delivered by any suitable method, including transfection, electroporation, liposome delivery, membrane fusion techniques, high velocity DNA-coated pellets, viral infection and protoplast fusion. The methods used to construct any embodiment of the compositions provided herein include genetic engineering, recombinant engineering, and synthetic techniques.

An engineered protein, a guide polynucleotide, or a polynucleotide encoding a composition provided herein can be delivered to a cell by electroporation. Electroporation using, for example, the NEON® Transfection System (ThermoFisher Scientific) or the Lonza Nucleofactor technologies also be used for delivery of nucleic acids and proteins into a cell. For example, an engineered protein provided herein can be purified and complexed with a suitable guide polynucleotide for delivery into a cell. Electroporation parameters can be adjusted to optimize delivery efficiency and/or cell viability. Electroporation devices can have multiple electrical wave form pulse settings such as exponential decay, time constant and square wave. Every cell type has a unique optimal Field Strength (E) that is dependent on the pulse parameters applied such as voltage, capacitance and resistance. Application of optimal field strength causes electropermeabilization through induction of transmembrane voltage, which allows nucleic acids to pass through the cell membrane. In some embodiments, the electroporation pulse voltage, the electroporation pulse width, number of pulses, cell density, and tip type can be adjusted to optimize transfection efficiency and/or cell viability.

### (3) Cells and Cell-Free Systems

Provided herein are cells comprising a system, a guide polynucleotide, a composition, or an engineered protein provided herein. In some embodiments, a polynucleotide encoding for an engineered protein provided herein and a polynucleotide encoding for a guide polynucleotide provided herein are administered to a cell or a population of cells.

The compositions, polynucleotides, engineered proteins, guide polynucleotides and systems provided herein can be delivered to any suitable cell. In some embodiments, compositions, polynucleotides, engineered proteins, guide polynucleotides and systems provided herein modulate a gene, a protein, and/or a functional phenotype of a cell.

Suitable cells can include but are not limited to eukaryotic and prokaryotic cells and/or cell lines. A suitable cell can be, for example, a human primary cell. A primary cell can be taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines. A primary cell can be acquired from a variety of sources such as an organ, vasculature, buffy coat, whole blood, apheresis, plasma, bone marrow, tumor, cell-bank, cryopreservation bank, or a blood sample. A primary cell can be a stem cell.

Suitable cells that can contacted with a composition, a polynucleotide, an engineered protein, a guide polynucleotide, or a system provided herein include but are not limited to: epithelial cells, fibroblast cells, neural cells, keratinocytes, hematopoietic cells, melanocytes, chondrocytes, leukocytes, lymphocytes (B, NK, and T), macrophages, monocytes, mononuclear cells, cardiac muscle cells, other muscle cells, granulosa cells, cumulus cells, epidermal cells, endothelial cells, pancreatic islet cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, Leydig cells, peritubular cells, Sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopaminergic cells, squamous epithelial cells, osteocytes, osteoblasts, osteoclasts, dopaminergic cells, embryonic stem cells, fibroblasts and fetal fibroblasts. Further, the one or more cells can be, for example, pancreatic islet cells and/or cell clusters or the like, including, but not limited to pancreatic α cells, pancreatic β cells, pancreatic δ cells, pancreatic F cells (also called Pancreatic polypeptide cells or PP cells), or pancreatic ε cells.

Suitable cells also include stem cells such as, by way of example, embryonic stem cells, induced pluripotent stem cells, hematopoietic stem cells, neuronal stem cells and mesenchymal stem cells. Suitable cells can comprise any number of primary cells, such as human cells, non-human cells, and/or mouse cells. Suitable cells can be progenitor cells. Suitable cells can be derived from the subject to be treated. For example, the subject to be treated can be a subject with a disease, a subject in need of treatment, or a subject that is immunocompromised. Suitable cells can be derived from a human donor.

In some embodiments, the cell is genetically modified by the methods, systems, and compositions provided herein. Cells provided herein can be administered to a subject in need thereof, for example, a subject with a disease or a condition in need of treatment.

A method of attaining suitable cells, such as human primary cells, can comprise selecting cells. In some embodiments, a cell can comprise a marker that can be selected for the cell. For example, such marker can comprise GFP, a resistance gene (for example, a gene conferring antibiotic resistance), a cell surface marker, an endogenous tag. Cells can be selected using any endogenous marker. Suitable cells can be selected using any technology. Such technology can comprise flow cytometry and/or magnetic columns. The selected cells can also be expanded to large numbers.

Delivery vehicles for in vivo and ex vivo use can include pharmaceutically acceptable carriers. Pharmaceutically acceptable carriers are determined in part by the particular composition being administered, for example, a polynucleotide, a protein, a vector, or a cell, as well as by the particular method used to administer the composition.

Provided herein are cell-free systems comprising a system, a composition, a guide polynucleotide, or an engineered protein provided herein. A cell-free system comprises components sufficient for a synthetic reaction and in some cases, retain DNA ligase and nickase bioactivity when stored under room temperature for a period of time. In some embodiments, the cell-free system comprises a set of reagents capable of providing for or supporting a biosynthetic reaction. Non-limiting examples of biosynthetic reactions include DNA replication, transcription, translation, or a combination of reactions in vitro in the absence of cells. Cell-free systems can be prepared using enzymes, coenzymes, and other subcellular components either isolated or purified from eukaryotic or prokaryotic cells, including recombinant cells, or prepared as extracts or fractions of such cells. A cell-free system can be derived from a variety of sources, including, but not limited to, eukaryotic and prokaryotic cells, such as bacteria including, but not limited to, E. coli, thermophilic bacteria and the like, wheat germ, rabbit reticulocytes, mouse L cells, Ehrlich's ascitic cancer cells, HeLa cells, CHO cells and budding yeast and the like. In some embodiments, the cell-free system is lyophilized. In some embodiments, the cell-free system further comprises a scaffold.

### (4) Pharmaceutical Compositions, Dosing, and Administration

Provided herein are pharmaceutical compositions comprising: a composition, a guide polynucleotide, an engineered protein, or a polynucleotide provided herein; and a pharmaceutically acceptable diluent, carrier, or excipient. Provided herein is a pharmaceutical composition comprising a vector provided herein; and a pharmaceutically acceptable diluent, carrier, or excipient.

In some embodiments, compositions provided herein, for example, a vector comprising a system or a composition provided herein, are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions and systems provided herein may be formulated in dosage unit form for ease of administration and uniformity of dosage. A unit dosage form is a physically discrete unit of a composition provided herein appropriate for a subject to be treated. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

Provided herein are pharmaceutical compositions for administering a composition or a system to a subject in need thereof. In some embodiments, the pharmaceutical composition is a treatment of a disease or a condition provided herein. In some embodiments, pharmaceutical compositions provided herein are in a form that allows for compositions provided herein to be administered to a subject. In some embodiments, the pharmaceutical composition is formulated for intratumoral delivery. In some embodiments, administration of a pharmaceutical composition provided herein is local administration or systemic administration. In some embodiments, a pharmaceutical composition provided herein is formulated for administration/for use in administration via an intratumoral, subcutaneous, intradermal, intramuscular, inhalation, intravenous, intraperitoneal, or intracranial route. In some embodiments, the administering is every 1, 2, 4, 6, 8, 12, 24, 36, or 48 hours. In some embodiments, the administering is at least about 5 hours, at least about 10 hours, at least about 12 hours, at least about 15 hours, at least about 20 hours, at least about 24 hours (1 day), at least about 48 hours (2 days), at least about 72 hours (3 days), at least about 96 hours (4 days), at least about 120 hours (5 days), at least about 144 hours (6 days), at least about 168 hours (7 days), at least about 336 hours (14 days), at least about 504 hours (21 days), at least about 672 hours (28 days), up to 744 hours (31 days). In some embodiments, the administering is every 744 hours (once per month) or once per year (365 days).

Vectors can be delivered in vivo by administration to an individual subject, typically by systemic administration, for example, intravenous, intraperitoneal, intramuscular, subdermal, or intracranial infusion. Vector can be delivered by topical application, as described below. Alternatively, vectors can be delivered to cells ex vivo, such as cells explanted from an individual subject, for example, lymphocytes, T cells, bone marrow aspirates, or tissue biopsy, followed by reimplantation of the cells into a subject, usually after selection for cells which have incorporated the vector. Prior to or after selection, the cells can be expanded in cell culture or in a bioreactor.

Provided herein are cells expressing a system or a composition provided herein. In some embodiments, the cell is contacted in vitro or ex vivo with a nucleic acid encoding for a composition or a system provided herein. In some embodiments, the cell is contacted in vitro or ex vivo with a vector encoding for an engineered protein provided herein, a system provided herein, or a composition provided herein.

### (5) Scaffolds and Systems

Provided herein are scaffolds, wherein the scaffolds comprise any composition, system, or guide polynucleotide provided herein. The scaffolds provided herein can be useful in the detection of newly edited nucleic acids or identifying a target nucleic acid provided herein. In some embodiments, the compositions provided herein are immobilized to the scaffold. In some embodiments, the scaffold comprises a surface. In some embodiments, the surface comprises a solid, a semi-solid, or a gel surface. In some embodiments, the scaffold comprises a reaction chip, a paper, a quartz microfiber, mixed esters of cellulose, a porous aluminum oxide, a patterned surface, a tube, a well, or a matrix. In some embodiments, the scaffold comprises a patterned surface suitable for immobilization of molecules in an ordered pattern. In some embodiments, a patterned surface refers to an arrangement of different regions in or on an exposed layer of a scaffold. In some embodiments, the scaffold comprises an array of wells or depressions in a surface. The composition and geometry of the scaffold can vary with its use. In some embodiments, the scaffold is a planar structure such as a slide, chip, microchip and/or array. As such, the surface of the scaffold can be in the form of a planar layer. In some embodiments, the scaffold comprises one or more surfaces of a flowcell. A flowcell is a type of chamber comprising a solid surface across which one or more fluid reagents can be flowed. In some embodiments, the scaffold or its surface is non-planar, such as the inner or outer surface of a tube or vessel. In some embodiments, the scaffold comprise microspheres or beads. Microspheres, beads, or particles can be made of various material including, but not limited to, plastics, ceramics, glass, and polystyrene. In some embodiments, the microspheres are magnetic microspheres or beads. Alternatively or additionally, the beads may be porous. The bead sizes range from nanometers (nm), for example, from about 100 nm, to millimeters, for example, about 1 mm.

In some embodiments, the scaffold comprises a set of engineered proteins, a set of polynucleotides provided herein, a set of systems provided herein, or any combination thereof. Provided herein are systems further a scaffold, wherein the scaffold comprises a surface. In some embodiments, the scaffold further comprises a cell-free system provided herein. In some embodiments, the systems and scaffolds provided herein further comprise reagents for nucleic acid amplification. In some embodiments, the systems and scaffolds provided herein further comprise reagents for DNA replication. In some embodiments, the systems provided herein further comprise: (a) a scaffold provided herein; (b) a reporter molecule; and (c) a detector. In some embodiments, when a target nucleic acid forms a complex with the scaffold, a guide polynucleotide provided herein, or an engineered protein provided herein, the reporter molecule produces a detectable signal that is detected by the detector. In some embodiments, the reporter molecule is selected from the group consisting of: a fluorophore, a dye, a polypeptide, an antibody, a nucleic acid, and any combination thereof. In some embodiments, the detectable signal is a calorimetric signal, a potentiometric signal, an amperometric signal, an optical signal, or a piezo-electric signal. A reporter molecule can be used to identify a cell comprising a new nucleic acid edited by the engineered protein provided herein. The reporter molecule can also be used for example, cell sorting, nucleic acid isolation, nucleic acid sequencing, or immunochemistry techniques.

### (6) Kits

Provided herein are kits, wherein the kits comprise: a system, a composition, a polynucleotide, a vector, a guide polynucleotide, or an engineered mRNA or protein provided herein; and packaging and materials therefor. In some embodiments, the kit further comprises a scaffold provided herein. In some embodiments, the kit further comprises a cell-free system. In some embodiments, the kit further comprises a population of cells. In some embodiments, the cells are stored in a cryopreservation medium. In some embodiments, the cryopreservation medium comprises: dimethyl sulfoxide (DMSO). In some embodiments, the cryopreservation medium comprises a buffer, an isotonic agent or an apoptosis inhibitor. Non-limiting examples of buffer elements include: citrate, phosphate, succinate, tartrate, fumarate, gluconate, oxalate, lactate, acetate, histidine and tris. Non-limiting examples of isotonic agents include, for example, citrate, phosphate, succinate, tartrate, fumarate, gluconate, oxalate, lactate, acetate, histidine, and tris. Additional isotonic agents include sodium chloride, potassium chloride, boric acid, sodium borate, mannitol, glycerin, propylene glycol, polyethylene glycol, maltose, sucrose, erythritol, arabitol, xylitol, sorbitol trehalose, and glucose. The apoptosis inhibitor can include, for example, a Rho associated kinase (ROCK) inhibitor, catalase, and zVAD-fmk. In some embodiments, the kits comprise reagents. In some embodiments, the reagents comprise saccharides and saccharide derivatives. In some embodiments, the saccharide derivatives comprise sodium carboxymethyl cellulose or cellulose acetate. In some embodiments, the reagents comprise detergents, glycols, polyols, esters, buffering agents, alginic acid, and/or organic solvents.

In some embodiments, a formulation of a composition described herein is prepared in a single container for administration to a cell, a cell-free system, or a subject. In some embodiments, a formulation of a composition provided herein is prepared two containers for administration, separating the guide polynucleotide or polynucleotide encoding the guide polynucleotide and/or the polynucleotide encoding the engineered protein provided herein. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some embodiments, the containers are RNase free.

Provided herein are kits comprising: a first container comprising: a donor nucleic acid, a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a ligation splint 2 region that has complementarity to the donor nucleic acid, the target nucleic acid and at least one mismatch nucleobase relative to the target nucleic acid; and (iv) a ligation splint 1 region, wherein the ligation splint 1 region comprises: a DNA nucleotide and an RNA nucleotide, and a second container comprising: an engineered protein or a polynucleotide encoding for the engineered protein, wherein the engineered protein comprises: a nickase operably linked to a DNA ligase. In some embodiments, a kit provided herein further comprises reagents for nucleic acid amplification, transcription, translation, or nucleic acid isolation. In some embodiments, a kit provided herein further comprises a reporter molecule provided herein.

### (7) Methods of Determining Gene Editing Activity

Provided herein are methods of determining gene editing activity, efficiency, and selectivity of an engineered protein, composition, or system provided herein for a target nucleic acid sequence. In some embodiments, the target nucleic acid is cleaved by a nuclease or a nickase provided herein upon binding of the guide polynucleotide to the target nucleic acid sequence. In some embodiments, the target nucleic acid is a DNA. In some embodiments, the nickase region of the engineered protein provided herein cleaves the DNA resulting in a single-stranded break. In some embodiments, the nickase region of the engineered protein provided herein cleaves the target DNA via a staggered DNA single or double-stranded break. The ability of a nuclease or a nickase provided herein to recognize a PAM sequence can be determined by an in vitro selection assay.

The activity of a system provided herein may be assayed using a cell expressing a reporter protein or containing a reporter gene. For example, a reporter gene may be engineered to contain an obstruction, such as a stop codon, a frameshift mutation, a spacer, a linker, or a transcriptional terminator; the system may then be used to remove the obstruction and the resultant functional reporter protein may be detected. Similarly, a reporter gene can be introduced into the target nucleic acid for detection of the target. In some embodiments, the reporter gene may be designed such that a specific sequence modification is required to restore functionality of the reporter protein. In other embodiments, the reporter gene may be designed such that any insertion or deletion which results in a frame shift of one or two bases in the target nucleic acid may be sufficient to restore functionality of the reporter protein. Examples of reporter proteins encoded by a reporter gene include colorimetric enzymes, metabolic enzymes, fluorescent proteins, enzymes and transporters associated with antibiotic resistance, and luminescent enzymes. Examples of such reporter proteins include β-galactosidase, Chloramphenicol acetyltransferase, Green fluorescent protein, Red fluorescent protein, and Firefly and Renilla luciferase. Different detection methods may be used for different reporter proteins. For example, the reporter protein may affect cell viability, cell growth, fluorescence, luminescence, or expression of a detectable product. In some embodiments, the reporter protein may be detected using a colorimetric assay. In some embodiments, the reporter protein may be a fluorescent protein, and DNA editing may be assayed by measuring the degree of fluorescence in treated cells, or the number of treated cells with at least a threshold level of fluorescence. In some embodiments, transcript levels of a reporter gene may be assessed. In other embodiments, a reporter gene may be assessed by sequencing.

Integration of the donor nucleic acid ligated by DNA ligase into the target nucleic acid can be measured using any technique, for example, integration can be measured by denaturing urea polyacrylamide gel electrophoresis, PAGE gel electrophoresis, flow cytometry, a surveyor nuclease assay, tracking of indels by decomposition (TIDE), junction PCR, droplet digital PCR, or any combination thereof. In other embodiments, transgene integration can be measured by PCR or droplet digital PCR. A TIDE analysis can also be performed on engineered cells. Ex vivo cell transfection can also be used for diagnostics, research, or for gene therapies. In some embodiments, the transfected cells are re-infused into the host organism. In some embodiments, cells are isolated from the subject organism, transfected with a nucleic acid and re-infused back into the subject.

The amount of genetically modified cells that can be necessary to be therapeutically effective in a subject can vary depending on the viability of the cells, and the efficiency with which the cells have been genetically modified. For example, the efficiency with which a transgene has been integrated into one or more cells can determine the number of cells administered to the subject. In some embodiments, the product (e.g., multiplication) of the viability of cells post genetic modification and the efficiency of integration of a transgene can correspond to the therapeutic aliquot of cells available for administration to a subject. In some embodiments, an increase in the viability of cells post-genetic modification can correspond to a decrease in the amount of cells that are necessary for administration to be therapeutically effective in a subject. In some embodiments, an increase in the efficiency with which a transgene has been integrated into one or more cells can correspond to a decrease in the amount of cells that are necessary for administration to be therapeutically effective in a subject. In some embodiments, determining an amount of cells that are necessary to be therapeutically effective can comprise determining a function corresponding to a change in the viability of cells over time. In some embodiments, determining an amount of cells that are necessary to be therapeutically effective can comprise determining a function corresponding to a change in the efficiency with which a transgene can be integrated into one or more cells with respect to time dependent variables. Variables can include, for example, cell culture time, electroporation time, and cell stimulation time.

Non-homologous end joining (NHEJ) and homology-directed repair (HDR) can be quantified using a variety of methods. For example, a percent of NHEJ, HDR, or a combination of both can be determined by co-delivering the gene editing molecules, for example a guide polynucleotide and an engineered protein provided herein, with a donor nucleic acid that encodes a promoter-less tag or marker into cells. In some embodiments, the target marker is a green fluorescent protein (GFP) of a polynucleotide encoding the GFP. After a duration of time, for example, about 72 to 96 hours, flow cytometry can be performed to quantify the total cell number (N_{Total}), tag-positive cell number and tag/GFP-negative cell number. Among the tag-negative cells, next-generation sequencing can be performed to identify cells without mutations and with mutations. HDR efficiency and NHEJ efficiency can be calculated from the assay.

Additional assays for determining gene editing efficiency of a system or composition provided herein can include but is not limited to: RT-PCR, nucleic acid sequencing, T7 endonuclease 1 (T7E1) mismatch detection assays, tracking of indels by decomposition (TIDE) assays, and indel detection by amplicon analysis (IDAA) assays. The indel pattern that is induced at the target site of a programmable nuclease or nickase may also be determined by PCR-amplifying the respective region and subsequent next generation sequencing. To obtain a quantitative single-cell view of gene editing efficiency, cell surface markers can be targeted and the loss of signal that occurs as a consequence of indel formation by flow cytometry can be quantified. Single-cell sequencing can be employed to assess the genome editing efficiency.

### (8) Applications

Provided herein are methods of modifying a target nucleic acid or generating an alteration in a target nucleic acid or a gene in a cell. In some embodiments, the methods comprise, administering to a cell, a tissue, or a subject the system provided herein, the composition provided herein, or a cell provided herein wherein the administering generates an alteration in a target nucleic acid. In some embodiments, the number of alterations in the target nucleic acid is at least 1 alteration. In some embodiments, the percentage of target nucleic acid molecule alteration is at 0.5%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 99%. In some embodiments, the method of modifying the target nucleic acid further comprises mutation of a target nucleic acid sequence. In some embodiments, the alteration or the modification of the target nucleic acid comprises: an insertion, a deletion, a substitution, a change in copy number, a point mutation, a frameshift mutation, a missense mutation, a nonsense mutation, a mutation in a stop codon, an epigenetic mark, or any combination thereof.

In some embodiments, the alteration is made in the target nucleic acid to form an edited nucleic acid. In some embodiments, the edited nucleic acid restores expression of a wild-type protein that is encoded by the gene relative to a comparable cell or population of cells that were not contacted with the system or the composition provided herein.

Provided herein are methods of ex vivo modifying a cell. In some embodiments, the methods comprise: contacting a cell with a composition provided herein under conditions that permit nuclease or nickase cleavage of a target nucleic acid molecule, thereby modifying said cell. Provided herein are methods of ex vivo modifying a cell. In some embodiments, the methods comprise: contacting a cell with a composition provided herein under conditions that permit DNA ligation of new nucleic acid sequence for incorporation into the target nucleic acid, thereby modifying said target nucleic acid and the cell.

Further provided herein are methods of ex vivo modifying a cell, the method comprising: contacting a cell with a ribonucleoprotein (RNP) complex, wherein the RNP complex comprises: (i) an engineered protein provided herein; and (ii) a polynucleotide that binds to a target nucleic acid, wherein upon contacting the cell with the RNP complex, the engineered protein cleaves a target nucleic acid molecule and ligates a new nucleic acid, thereby modifying said cell. In some embodiments, the cell is an immune cell or a stem cell. In some embodiments, the immune cell is a leukocyte, a lymphocyte, a natural killer cell, a dendritic cell, a macrophage, a myeloid cell, a T-cell, a B cell, a stem cell, an induced-pluripotent derived cell, a cancer cell, or an endothelial cell. In some embodiments, the stem cell is an embryonic stem cell, an induced-pluripotent stem cell (iPSC), or an adult stem cell.

Methods of modifying a target nucleic acid provided herein can be used for agricultural applications, for example, the generation of improved plant species. Methods of modifying a target nucleic acid provided here can also be used in biomedical applications, drug screening platforms, and therapeutic treatments. For example, the compositions and systems provided herein can be used to produce cell therapies for the treatment of a disease or a disorder.

Provided herein are methods of modifying a nucleic acid, the method comprising: contacting a cell or a cell-free system with: (a) a donor nucleic acid; (b) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nickase; and (iii) a ligation splint 2 region that has complementarity to the donor nucleic acid, the target nucleic acid and at least one mismatch nucleobase relative to the target nucleic acid; and (iv) a ligation splint 1 region, wherein the ligation splint 1 region comprises: a deoxyribonucleotide and a ribonucleotide; and (c) an engineered protein or a polynucleotide encoding the engineered protein, wherein the engineered protein comprises: (i) a nickase region; and (ii) a DNA ligase region; wherein: the guide polynucleotide forms a complex with the engineered protein via the protein binding region, the targeting sequence forms a complex with the complementary strand of the target nucleic acid, the nickase region of the engineered protein generates a break in the target nucleic acid to generate a leading strand, the ligation splint 1 region forms a complex with the leading strand and the ligation splint 2 region forms a complex with donor nucleic acid, and wherein the DNA ligase region catalyzes the formation of a phosphodiester bond between the 3′ hydroxyl end of the leading strand and the 5′ phosphate end of the donor nucleic acid, thereby inserting a new nucleic acid. In some embodiments, the targeting region dissociates from the complementary strand. In some embodiments, the new nucleic acid is incorporated into the target nucleic acid by hybridizing to the complementary strand. In some embodiments, the incorporation of the donor nucleic acid recruits a DNA repair protein to the target nucleic acid that edits a nucleobase of the target nucleic acid.

Provided herein are methods of detecting a nucleic acid in a test sample, the method comprising: (a) immobilizing a guide polynucleotide onto a scaffold provided herein; and (b) contacting the guide polynucleotide with: (i) a test sample; and (ii) an engineered protein provided herein. In some embodiments the test sample comprises a target nucleic acid capable of binding to the guide polynucleotide. In some embodiments, a complex is formed between the guide polynucleotide, an engineered protein provided herein, and the target nucleic acid. In some embodiments, upon formation of the complex the engineered protein (e.g., the nickase region) cleaves the target nucleic acid. In some embodiments, the method further comprises detecting a signal indicating cleavage of the target nucleic acid molecule or detecting a new nucleic acid strand comprising a reporter molecule, thereby detecting a target nucleic acid in the sample.

In some embodiments, prior to the detecting step, the method further comprises, amplifying the target nucleic acid. In some embodiments, the amplifying comprises polymerase chain reaction (PCR), nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), multiple displacement amplification (MDA), rolling circle amplification (RCA), improved multiple displacement amplification (EVIDA), 1 simple method amplifying RNA targets (SMART), single primer isothermal amplification (SPIA), ligase chain reaction (LCR), transcription mediated amplification (TMA), ramification amplification method (RAM), or any combination thereof. In some embodiments, the methods further comprise performing an endonuclease mismatch detection assay, an immunoassay, gel electrophoresis, a plasmid interference assay, nucleic acid sequencing, or any combination thereof. In some embodiments, the detecting comprises calorimetric detection, potentiometric detection, amperometric detection, optical detection, piezo-electric detection, or any combination thereof.

Provided herein are methods of treating a disease or a condition in a subject in need thereof. In some embodiments, the subject has, is suspected of having, or is diagnosed with a disease or a condition. In some embodiments, the methods comprise administering to a subject a system, composition, vector, pharmaceutical composition, or polynucleotide, provided herein. In some embodiments, the administering is local or systemic. In some embodiments, the administering is intranasal administration, subcutaneous administration, intravenous administration, inhalation, intramuscular administration, intratumoral administration, peritumoral administration, intrathecal administration, vaginal administration, or intradermal administration. In some embodiments, the method further comprises administering to the subject a therapeutic agent.

Provided is the use of the compositions and systems provided herein in the manufacture of a medicament. Also provided is the use of the compositions described herein in the manufacture of a medicament for therapeutic and/or prophylactic treatment of a disease or condition described herein. In some embodiments, a disease or a condition is caused by a mutated disease-associated gene. In some embodiments, a disease-associated gene is any gene associated with an increase in the risk of having or developing a disease. In some embodiments, a disease-associated gene is any gene or polynucleotide which is yielding transcription or translation products at an abnormal level or in an abnormal form in cells derived from a disease-affected tissues compared with tissues or cells of a non-disease control. In some embodiments, a disease-associated gene is a gene that becomes expressed at an abnormally high level. In some embodiments, a disease-associated gene is a gene that becomes expressed at an abnormally low level, where the altered expression correlates with the occurrence and/or progression of the disease. In some embodiments, a disease-associated gene is a gene possessing mutation(s) or genetic variation that is responsible or is in linkage disequilibrium with a gene(s) that is responsible for the etiology of a disease. The transcribed or translated products may be known or unknown, and may be at a normal or abnormal level.

In some embodiments, a disease or a condition is caused by mutations associated with DNA repeat instability and neurological disorders. Specific aspects of tandem repeat sequences have been found to be responsible for more than twenty human diseases. The system may be harnessed to correct these defects of genomic instability. In some embodiments, the disease or the condition is a neurological disease, a cardiovascular disease, cancer, a respiratory disease, diabetes, obesity, an eye disease, loss of hearing, blindness, or a rare genetic disease or disorder. In some embodiments, the disease or the condition is age-related macular degeneration, a schizophrenic disorder, trinucleotide repeat disorder, Fragile X Syndrome. In some embodiments, the disease or the condition is a Secretase Related Disorder. In some embodiments, the disease or the condition is a Prion-related disorder. In some embodiments, the disease or the condition is ALS. In some embodiments, the disease or the condition is a drug addiction. In some embodiments, the disease or the condition is Autism. In some embodiments, the disease or the condition is Alzheimer's Disease. In some embodiments, the disease or the condition is inflammation. In some embodiments, the disease or the condition is Parkinson's Disease. Further examples of diseases and conditions treatable with the systems and compositions provided herein include but are not limited to: Aieardi-Goutieres Syndrome; Alexander Disease; Allan-Herndon-Dudley Syndrome; POLG-Related Disorders; Alpha-Mannosidosis (Type II and III); Alstrom Syndrome; Angelman; Syndrome; Ataxia-Telangiectasia; Neuronal Ceroid-Lipofuscinoses; Beta-thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders, Genetic Prion Diseases; Dravet Syndrome; Early-Onset Familial Alzheimer Disease; Friedreich's Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders, Urea Cycle Disorders; COL 1 A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type 11 (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum. In some embodiments, the subject is a mammal. In some embodiments, the subject is a human.

### EXEMPLARY EMBODIMENTS

Provided herein are compositions comprising: (a) a donor nucleic acid; (b) a polynucleotide encoding a protein construct comprising a nickase or a variant thereof and a DNA ligase or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: a deoxyribonucleotide and a ribonucleotide, wherein the ligation splint 1 region has complementarity to the target nucleic acid. Provided herein are compositions, wherein the ligation splint 1 region forms a DNA-RNA (DR)-loop upon association with the target nucleic acid and a DNA ligase. Provided herein are compositions, wherein the ligase splint 1 region comprises a ratio of RNA to DNA of 1:1 up to 20:1. Provided herein are compositions, wherein the ligation splint 1 region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18 or 20:1. Provided herein are compositions, wherein the ligation splint 2 region comprises at least about 5 nucleotides up to 10,000 nucleotides. Provided herein are compositions, wherein the ligation splint 2 region comprises at least about 7 nucleotides up to 1,000 nucleotides. Provided herein are compositions, wherein the ligation splint 1 region comprises at least about 5 nucleotides up to 20 nucleotides. Provided herein are compositions, wherein the ligation splint 2 region comprises a reverse complement sequence of a non-coding polynucleotide sequence or a variant thereof. Provided herein are compositions, wherein the ligation splint 2 region comprises a reverse complement sequence of a sequence encoding a coding region of a polynucleotide sequence or a variant thereof. Provided herein are compositions, wherein the ligation splint 2 region comprises a reverse complement sequence of a sequence encoding for an exon or an intron. Provided herein are compositions, wherein the ligation splint 2 region comprises a complement sequence of a sequence encoding a non-coding polynucleotide sequence or a variant thereof. Provided herein are compositions, wherein the ligation splint 2 region comprises a complement sequence of a sequence encoding a coding region of a polynucleotide sequence or a variant thereof. Provided herein are compositions, wherein the ligation splint 2 region comprises a complement sequence of a sequence encoding for an exon or an intron. Provided herein are compositions, wherein the ligation splint 2 region comprises a sequence comprising at least one nucleobase that is complementary to or mismatched with a sequence encoding a splice acceptor site. Provided herein are compositions, wherein the nickase is an engineered Cas protein or a functional variant thereof. Provided herein are compositions, wherein the engineered Cas protein or a functional variant thereof comprises at least one amino acid substitution at position 840 corresponding to SEQ ID NO: 69. Provided herein are compositions, wherein the engineered Cas protein or a functional variant thereof further comprises at least one amino acid substitution at position 221, 394, or a combination thereof, corresponding to SEQ ID NO: 69. Provided herein are compositions, wherein the engineered Cas protein or a functional variant thereof comprises amino acid substitutions of R221K, N394K, H840A, or any combination thereof. Provided herein are compositions, wherein the engineered Cas protein or a functional variant thereof comprises a sequence 97%, 98%, 99%, or 100% identical to SEQ ID NO: 70 or SEQ ID NO: 71. Provided herein are compositions, wherein the engineered Cas protein or a functional variant thereof comprises a sequence of SEQ ID NO: 70 or SEQ ID NO: 71. Provided herein are compositions, wherein the DNA ligase or a functional fragment comprises an E. coli DNA ligase, a Taq DNA ligase, a T3 DNA ligase, a T4 DNA ligase, a T7 DNA ligase, a Chlorella virus DNA ligase, a human DNA ligase I, a human DNA ligase II, a human DNA ligase III, a human DNA ligase IV, a human ligase V, a variant, or a combination thereof. Provided herein are compositions, wherein the DNA ligase or a functional fragment thereof comprises Chlorella virus DNA ligase, a T4 DNA ligase, or a human DNA ligase IV. Provided herein are compositions, wherein the DNA ligase or a functional fragment thereof comprises a sequence 90%, 91%, 92%, 93%, 94%, 95%, 97%, 98%, 99%, or 100% identical to SEQ ID NOS: 58-66. Provided herein are compositions, wherein the DNA ligase or a functional fragment thereof comprises a sequence of SEQ ID NOS: 58-66. Provided herein are compositions, wherein the protein construct further comprises a linker, a nuclear localization sequence (NLS), or a combination thereof. Provided herein are compositions, wherein the DNA ligase or a functional fragment thereof is linked to the nickase or a variant thereof. Provided herein are compositions, wherein the protein construct comprises a sequence 90%, 91%, 92%, 93%, 94%, 95%, 97%, 98%, 99%, or 100% identical to SEQ ID NOS: 89-101.

Provided herein are engineered fusion proteins, wherein the engineered fusion proteins comprise: (a) a DNA ligase or a functional fragment thereof; and (b) an engineered nickase that comprises three amino acid substitutions at positions corresponding to amino acid positions 221, 394, and 840 of a nuclease comprising a sequence of SEQ ID NO: 69. Provided herein are engineered fusion proteins, wherein the substitutions result in enhanced nickase activity as compared to an otherwise equivalent engineered nickase. Provided herein are engineered fusion proteins, wherein the engineered nickase comprises amino acid substitutions of R221K, N394K, and H840A. Provided herein are engineered fusion proteins, wherein the engineered nickase or a functional variant thereof comprises a sequence 97%, 98%, 99%, or 100% identical SEQ ID NO: 71. Provided herein are engineered fusion proteins, wherein the engineered nickase or a functional variant thereof comprises a sequence of SEQ ID NO: 71. Provided herein are engineered fusion proteins, wherein the DNA ligase or a functional fragment comprises an E. coli DNA ligase, a Taq DNA ligase, a T3 DNA ligase, a T4 DNA ligase, a T7 DNA ligase, a Chlorella virus DNA ligase, a human DNA ligase I, a human DNA ligase II, a human DNA ligase III, a human DNA ligase IV, a human ligase V, a variant, or a combination thereof. Provided herein are engineered fusion proteins, wherein the DNA ligase or a functional fragment thereof comprises Chlorella virus DNA ligase, a T4 DNA ligase, or a human DNA ligase IV. Provided herein are engineered fusion proteins, wherein the DNA ligase or a functional fragment thereof comprises a sequence 97%, 98%, 99%, or 100% identical to SEQ ID NO: 58, SEQ ID NO: 59, SEQ ID NO: 61, or SEQ ID NO: 65. Provided herein are engineered fusion proteins, wherein the DNA ligase or a functional fragment thereof comprises a sequence of SEQ ID NO: 58, SEQ ID NO: 59, SEQ ID NO: 61, or SEQ ID NO: 65. Provided herein are engineered fusion proteins, wherein the engineered fusion proteins further comprise a linker, a nuclear localization sequence (NLS), or an additional protein construct. Provided herein are engineered fusion proteins, wherein the engineered fusion proteins comprise an amino acid sequence 97%, 98%, 99%, or 100% identical to any one of SEQ ID NOS: 89-101. Provided herein are engineered fusion proteins, wherein the engineered fusion proteins comprise comprising an amino acid sequence of any one of SEQ ID NOS: 89-101. Provided herein are engineered fusion proteins, wherein the additional protein construct comprises a cell-targeting moiety, a receptor-targeting moiety, a regulatory element, a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof.

Provided herein are nucleic acids, wherein the nucleic acid encodes an engineered fusion protein provided herein. Provided herein are nucleic acids, wherein the nucleic acid encoding the engineered fusion protein comprises RNA. Provided herein are nucleic acids, wherein the nucleic acid encoding the engineered fusion protein comprises DNA.

Provided herein are vectors comprising a nucleic acid provided herein. Provided herein are vectors wherein the vector comprises a viral vector. Provided herein are vectors wherein the viral vector comprises a lentiviral vector, a retroviral vector, an adeno-associated viral vector (AAV), an adenoviral vector, a herpes simplex viral vector, an alphaviral vector, a flaviviral vector, a rhabdoviral vector, a measles viral vector, a Newcastle disease viral vector, a poxviral vector, a picornaviral vector, or an oncolytic viral vector.

Provided herein are systems for modifying a target nucleic acid, the systems comprising: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region has complementarity to the target nucleic acid, wherein upon introduction to a cell or a cell-free system, the system incorporates the donor nucleic acid into the target nucleic acid, thereby modifying the target nucleic acid. Provided herein are systems for modifying a target nucleic acid, the systems comprising: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: one or more ribonucleotides or one or more deoxyribonucleotides, wherein the ligation splint 1 region has complementarity to the target nucleic acid, wherein upon introduction to a cell or a cell-free system, the system incorporates the donor nucleic acid into the target nucleic acid, thereby modifying the target nucleic acid. Provided herein are systems for modifying a target nucleic acid, the systems comprising: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: one or more deoxyribonucleotides, wherein the ligation splint 1 region has complementarity to the target nucleic acid, wherein upon introduction to a cell or a cell-free system, the system incorporates the donor nucleic acid into the target nucleic acid, thereby modifying the target nucleic acid. Provided herein are systems for modifying a target nucleic acid, wherein the systems comprise: (a) a donor nucleic acid; (b) an engineered protein construct comprising a nickase region; (c) a DNA ligase or a functional fragment thereof; and (d) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a ligation splint 2 region, wherein the ligation splint region has complementarity to the donor nucleic acid and has complementarity to the target nucleic acid, and wherein the ligation splint region comprises at least one alteration relative to the target nucleic acid; and (iv) a ligation splint 1 region, wherein the ligation splint 1 region comprises: a deoxyribonucleotide and a ribonucleotide, wherein the ligation splint 1 region has complementarity to the target nucleic acid, wherein upon introduction to a cell or a cell-free system, the system incorporates the donor nucleic acid into the target nucleic acid, thereby modifying the target nucleic acid.

Further provided herein are systems, wherein the ligation splint 1 region forms a DNA-RNA (DR)-loop upon association with the target nucleic acid and the DNA ligase. Further provided herein are systems, wherein the ligation splint 1 region comprises a ratio of ribonucleic acids (RNAs) to deoxyribonucleic acids (DNAs) of: 1:1 up to 20:1. Further provided herein are systems, wherein the ligation splint 1 region comprises a ratio of ribonucleic acids (RNAs) to deoxyribonucleic acids (DNAs) of: 1:0 up to 20:0. Further provided herein are systems, wherein the ligation splint 1 region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, or 20:1. Further provided herein are systems, wherein the ligation splint 1 region hybridizes to a leading strand upon cleavage of the target nucleic acid by the engineered protein construct. Further provided herein are systems, wherein the ribonucleotides and/or deoxyribonucleotides of the ligation splint 1 region hybridize to the leading strand. Further provided herein are systems, wherein the ligation splint 2 region comprises at least about 5 nucleotides up to 10,000 nucleotides. Further provided herein are systems, wherein the ligation splint 2 region comprises at least about 7 nucleotides up to 1,000 nucleotides. Further provided herein are systems, wherein the ligation splint 1 region comprises at least about 5 nucleotides up to 20 nucleotides. Further provided herein are systems, wherein the ligation splint 2 region comprises a reverse complement sequence of a non-coding polynucleotide sequence or a variant thereof. Further provided herein are systems, wherein the ligation splint 2 region comprises a reverse complement sequence of a sequence encoding a coding region of a polynucleotide sequence or a variant thereof. Further provided herein are systems, wherein the ligation splint 2 region comprises a reverse complement sequence of a sequence encoding for an exon or an intron. Further provided herein are systems, wherein the ligation splint 2 region comprises a complement sequence of a sequence encoding a non-coding polynucleotide sequence or a variant thereof. Further provided herein are systems, wherein the ligation splint 2 region comprises a complement sequence of a sequence encoding a coding region of a polynucleotide sequence or a variant thereof. Further provided herein are systems, wherein the ligation splint 2 region comprises a complement sequence of a sequence encoding for an exon or an intron. Further provided herein are systems, wherein the ligation splint 2 region comprises a sequence comprising at least one nucleobase that is complementary to or mismatched with a sequence encoding a splice acceptor site. Further provided herein are systems, wherein the targeting region hybridizes to a complementary strand of the target nucleic acid and the nickase region cleaves the target nucleic acid. Further provided herein are systems, wherein the targeting region hybridizes to the complementary strand of the target nucleic acids within at least about 5 nucleotides of a PAM sequence, at least about 10 nucleotides of a PAM sequence, at least about 15 nucleotides of a PAM sequence, or at least about 20 nucleotides of a PAM sequence. Further provided herein are systems, wherein the engineered protein construct comprises a Cas protein or a mutant Cas protein. Further provided herein are systems, wherein the Cas protein or the mutant Cas protein is a Type V Cas protein. Further provided herein are systems, wherein the Type V Cas protein comprises: a Cas12a, a Cas12b, a Cas12c, a Cas12d, a Cas12e, a Cas14, a Cas12g, a Cas12h, a Cas12i, a Cas12j, a Cas12k. Further provided herein are systems, wherein the Cas protein or the mutant Cas protein comprises: a Cas1, a Cas1B, a Cas2, a Cas3, a Cas4, a Cas5, a Cas6, a Cas7, a Cas8, a Cas9, a Cas10, a Cas11, a Cas12, a Cas13, a Cas14, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx1S, a Csf1, a Csf2, a CsO, a Csf4, a c2c1, a c2c3, a Cas9HiFi, an xCas9, a CasX, a CasY, a CasRX, a SpCas9-VQR, a SpCas9-VRQR, a SpCas9-VRER, a SaCas9-KKH, a SpCas9-NG, a SpCas9-NRRH, a SpCas9-NRTH, a SpCas9-NRCH, a iSpyMac, a St1Cas9 LMD9-LMG18311, a St1Cas9 LMD9-CNRZ1066, a St1Cas9-KQKL, a variant, or any combination thereof. Further provided herein are systems, wherein the Cas protein or the mutant Cas protein is a Type II Cas protein. Further provided herein are systems, wherein the Type II Cas protein comprises: a Cas9, a Cas1, a Cas2, or a Csn2. Further provided herein are systems, wherein engineered protein construct cleaves the target nucleic acid 5′ upstream of a PAM sequence. Further provided herein are systems, wherein the engineered protein construct cleaves the target nucleic acid to generate two single strands of DNA or two single strands of an RNA duplex. Further provided herein are systems, wherein the engineered protein construct generates a single-stranded break in the target nucleic acid. Further provided herein are systems, wherein the DNA ligase or the functional fragment thereof comprises an E. coli DNA ligase, a Taq DNA ligase, a T3 DNA ligase, a T4 DNA ligase, a T7 DNA ligase, a Chlorella virus DNA ligase, a human DNA ligase I, a human DNA ligase II, a human DNA ligase III, a human DNA ligase IV, a human ligase V, a variant, or a combination thereof. Further provided herein are systems, wherein the DNA ligase or the functional fragment thereof comprises an Archaeoglobus fulgidus DNA ligase, an Archaeoglobus fulgidus DNA ligase, a Pyrococcus furiosus DNA ligase, a Methanothermobacter thermautotrophicus DNA ligase, a Pyrococcus abyssi DNA ligase, a Sulfolobus solfataricus DNA ligase, a Haemophilus influenzae DNA Ligase, or a Herpes Simplex Virus (HSV) DNA ligase. Further provided herein are systems, wherein the ligation splint 2 region hybridizes to the donor nucleic acid. Further provided herein are systems, wherein the DNA ligase does not bind to the target nucleic acid. Further provided herein are systems, wherein the DNA ligase attaches the donor nucleic acid to the leading strand. Further provided herein are systems, wherein the new DNA strand has at least 50% up to 99.99% complementarity to the target nucleic acid sequence. Further provided herein are systems, wherein the new DNA strand comprises a mismatched nucleobase relative to a complementary strand of the target nucleic acid. Further provided herein are systems, wherein the secondary structure of the protein binding region of the guide polynucleotide comprises: a bulge, a stem, a loop, a hairpin, a wobble base pair, a pseudoknot, or a combination thereof. Further provided herein are systems, wherein the target nucleic acid comprises DNA. Further provided herein are systems, wherein the target nucleic acid comprises RNA. Further provided herein are systems, wherein the systems further comprising an additional protein construct, a linker, or a nuclear localization sequence (NLS). Further provided herein are systems, wherein the protein construct and the DNA ligase are linked together by a polypeptide linker.

Provided herein are compositions comprising: wherein the compositions comprise: a system provided herein, an engineered protein construct provided herein, a DNA ligase provided herein or a functional fragment thereof, or a guide polynucleotide provided herein.

Provided herein are compositions, wherein the compositions comprise: the systems provided herein; and a delivery vehicle.

Provided herein are polynucleotides, wherein the polynucleotides encode for a system provided herein, an engineered protein provided herein, or a guide polynucleotide provided herein.

Provided herein are sets of polynucleotides, wherein the sets of polynucleotides encode for a system provided herein, an engineered protein provided herein, or a guide polynucleotide provided herein.

Provided herein are nanoparticles, wherein the nanoparticles comprise: the polynucleotides provided herein, the sets of polynucleotides provided herein, the systems provided herein, the compositions provided herein, the cells provided herein, the vectors provided herein or any portion thereof. Further provided herein are nanoparticles, wherein the nanoparticles are lipid nanoparticles.

Provided herein are vectors, wherein the vectors comprise a polynucleotide provided herein or the set of polynucleotides provided herein.

Provided herein are cells, wherein the cells comprise: a system provided herein, an engineered proteins provided herein, a composition provided herein, a vector provided herein, or a guide polynucleotide provided herein.

Provided herein are compositions, wherein the compositions comprise: a donor nucleic acid, and a guide polynucleotide or a polynucleotide encoding the guide polynucleotide, wherein the guide polynucleotide comprises in 5′ to 3′ order: (a) a targeting region that has complementarity to a target nucleic acid; (b) a protein binding region, wherein the protein binding region comprises a secondary structure that binds to a protein; (c) a ligation splint 2 region, wherein the ligation splint 2 region has complementarity to the donor nucleic acid and has complementarity to the target nucleic acid and at least one alteration relative to the target nucleic acid or at least one nucleic acid strand thereof; and (d) a ligation splint 1 region. Provided herein are compositions, wherein the compositions comprise: a donor nucleic acid, and a guide polynucleotide or a polynucleotide encoding the guide polynucleotide, wherein the guide polynucleotide comprises in 5′ to 3′ order: (a) a targeting region that has complementarity to a target nucleic acid; (b) a protein binding region, wherein the protein binding region comprises a secondary structure that binds to a protein; (c) a ligation splint 2 region, wherein the ligation splint 2 region has complementarity to the donor nucleic acid and has complementarity to the target nucleic acid and at least one alteration relative to the target nucleic acid or at least one nucleic acid strand thereof; and (d) a ligation splint 1 region, wherein the ligation splint 1 region comprises one or more ribonucleotides or one or more deoxyribonucleotides. Provided herein are compositions, wherein the compositions comprise: a donor nucleic acid, and a guide polynucleotide or a polynucleotide encoding the guide polynucleotide, wherein the guide polynucleotide comprises in 5′ to 3′ order: (a) a targeting region that has complementarity to a target nucleic acid; (b) a protein binding region, wherein the protein binding region comprises a secondary structure that binds to a protein; (c) a ligation splint 2 region, wherein the ligation splint 2 region has complementarity to the donor nucleic acid and has complementarity to the target nucleic acid and at least one alteration relative to the target nucleic acid or at least one nucleic acid strand thereof; and (d) a ligation splint 1 region, wherein the ligation splint 1 region comprises one or more deoxyribonucleotides. Provided herein are compositions, wherein the compositions comprise: a donor nucleic acid, and a guide polynucleotide or a polynucleotide encoding the guide polynucleotide, wherein the guide polynucleotide comprises in 5′ to 3′ order: (a) a targeting region that has complementarity to a target nucleic acid; (b) a protein binding region, wherein the protein binding region comprises a secondary structure that binds to a protein; (c) a ligation splint 2 region, wherein the ligation splint 2 region has complementarity to the donor nucleic acid and has complementarity to the target nucleic acid and at least one alteration relative to the target nucleic acid or at least one nucleic acid strand thereof; and (d) a ligation splint 1 region, wherein the ligation splint 1 region comprises a deoxyribonucleotide and a ribonucleotide. Further provided herein are compositions, wherein the target nucleic acid comprises a double stranded DNA (dsDNA) or a single-stranded RNA (ssRNA) duplex that is cleaved by a nickase. Further provided herein are compositions, wherein the protein binding region binds to a nickase or an endonuclease. Further provided herein are compositions, wherein the secondary structure comprises a bulge, a stem, a loop, a hairpin, a wobble base pair, a pseudoknot, or a combination thereof. Further provided herein are compositions, wherein the ligation splint 2 region comprises at least about 5 nucleotides up to 10,000 nucleotides. Further provided herein are compositions, wherein the ligation splint 2 region comprises at least about 7 nucleotides up to 1,000 nucleotides. Further provided herein are compositions, wherein the ligation splint 2 region comprises at least on alteration that is a mismatched nucleobase. Further provided herein are compositions, wherein the mismatched nucleobase comprises an A/C mismatch, a A/T mismatch, an A/G mismatch, and T/C mismatch, a T/G mismatch, a T/A mismatch, a C/G mismatch, a C/A mismatch, a C/T mismatch, a G/C mismatch, a G/T mismatch, a G/A mismatch, or a combination thereof relative to the target nucleic acid. Further provided herein are compositions, wherein the donor nucleic acid comprises a reverse complement sequence of a non-coding region of a polynucleotide or a variant thereof. Further provided herein are compositions, wherein the donor nucleic acid comprises a reverse complement sequence of a sequence encoding a coding region of a polynucleotide or a variant thereof. Further provided herein are compositions, wherein the donor nucleic acid comprises a reverse complement sequence of a sequence encoding for an exon or an intron. Further provided herein are compositions, wherein the donor nucleic acid comprises a sequence comprising at least one nucleobase that is complementary to or mismatched with a sequence encoding a splice acceptor site. Further provided herein are compositions, wherein the donor nucleic acid comprises a complement sequence of a sequence encoding an intron or a variant thereof. Further provided herein are compositions, wherein the donor nucleic acid comprises a complement sequence of a sequence encoding an exon or a variant thereof. Further provided herein are compositions, wherein the donor nucleic acid comprises a complement sequence of a sequence encoding for an exon and an intron. Further provided herein are compositions, wherein the ligation splint 1 region forms a DNA-RNA (DR)-loop upon association with the target nucleic acid and a DNA ligase. Further provided herein are compositions, wherein the ligase splint 1 region comprises a ratio of RNA to DNA of 1:1 up to 20:1. Further provided herein are compositions, wherein the ligase splint 1 region comprises a ratio of RNA to DNA of 1:0 up to 20:0. Further provided herein are compositions, wherein the ligation splint 1 region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18 or 20:1. Further provided herein are compositions, wherein the ligation splint 1 region hybridizes to the target nucleic acid upon cleavage of the target nucleic acid by a nickase, wherein the nickase generates a leading strand and a complementary strand. Further provided herein are compositions, wherein the ribonucleotides hybridize to the leading strand. Further provided herein are compositions, wherein the ligation splint 1 region comprises at least about 5 nucleotides up to 10,000 nucleotides. Further provided herein are compositions, wherein the ligation splint 1 region comprises at least about 7 nucleotides up to 1,000 nucleotides. Further provided herein are compositions, wherein the ligation splint 1 region comprises at least about 5 nucleotides up to 20 nucleotides. Further provided herein are compositions, wherein the ligation splint 1 region comprises a reverse complement sequence encoding for an intron or a variant thereof. Further provided herein are compositions, wherein the ligation splint 1 region comprises a reverse complement sequence of a sequence encoding an exon or a variant thereof. Further provided herein are compositions, wherein the ligation splint 1 region comprises a reverse complement sequence of a sequence encoding an exon and an intron. Further provided herein are compositions, wherein the ligation splint 1 region comprises a sequence encoding for a splice acceptor site or nucleotide complementary to the splice acceptor site. Further provided herein are compositions, wherein the ligation splint 1 region comprises a complement sequence of a sequence encoding an intron or a variant thereof. Further provided herein are compositions, wherein the ligation splint 1 region comprises a complement sequence of a sequence encoding an exon or a variant thereof. Further provided herein are compositions, wherein the ligation splint 1 region comprises a complement sequence of a sequence encoding an exon and an intron. Further provided herein are compositions, wherein the ligation splint 1 region comprises a mismatched nucleobase relative to a splice acceptor site in a sequence of the target nucleic acid. Further provided herein are compositions, wherein the ribonucleotides are on the 3′ end of the ligation splint 1 region. Further provided herein are compositions, wherein the ligation splint 1 region comprises 1 ribonucleotide, 2 ribonucleotides, 3 ribonucleotides, 4 ribonucleotides, 5 ribonucleotides, or up to 10 ribonucleotides. Further provided herein are compositions, wherein the ligation splint 1 region is at least about 5 nucleotides up to 10 nucleotides in length Further provided herein are compositions, wherein the compositions further comprise an engineered protein or a polypeptide encoding the engineered protein. Further provided herein are compositions, wherein the engineered protein comprises: (a) a nickase region; and (b) a DNA ligase region. Further provided herein are compositions, wherein the DNA ligase region binds to the ligation splint 1 region or the ligation splint 2 region of the guide polynucleotide. Further provided herein are compositions, wherein the DNA ligase region ligates the donor nucleic acid to a 3′ end of the leading strand. Further provided herein are compositions, wherein the nickase region comprises a mutant Cas protein that generates a single-stranded break in a target nucleic acid. Further provided herein are compositions, wherein the Cas protein comprises: a Cas1, a Cas1B, a Cas2, a Cas3, a Cas4, a Cas5, a Cas6, a Cas7, a Cas8, a Cas9, a Cas10, a Cas11, a Cas12, a Cas13, a Cas14, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx1S, a Csf1, a Csf2, a CsO, a Csf4, a c2c1, a c2c3, a Cas9HiFi, an xCas9, a CasX, a CasY, a CasRX, a variant, or any combination thereof. Further provided herein are compositions, wherein the compositions further comprises a delivery vehicle. Further provided herein are compositions, wherein the delivery vehicle comprises a vector, a lipid, a nanoparticle, a plasmid, a virus, a liposome, an extracellular vesicle, an emulsion, a peptide, a carbohydrate, a polymer, chitosan, polyethylenimine (PEI), Poly (lactide-co-glycolide) (PLGA), Poly-L-lysine (PLL), or a combination thereof.

Provided herein are methods of ligating a donor nucleic acid with a target nucleic acid, wherein the methods comprise: contacting a cell or a cell-free system with: (a) a donor nucleic acid, (b) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a ligation splint 2 region, wherein the ligation splint 2 region is complementary to the donor nucleic acid and has complementarity to the target nucleic acid and at least one alteration relative to the target nucleic acid; (iv) a ligation splint 1 region, wherein the ligation splint 1 region comprises one or more deoxyribonucleotides or one or more ribonucleotides. (c) an engineered protein or a polynucleotide encoding the engineered protein, wherein the engineered protein comprises: (i) a nickase region; and (ii) a DNA ligase region; wherein: the guide polynucleotide forms a complex with the engineered protein via the protein binding region, the targeting sequence forms a complex with the complementary strand, the nickase region of the engineered protein generates a break in the target nucleic acid to generate a leading strand and a complementary strand, the ligation splint 1 region forms a complex with the leading strand, and wherein the DNA ligase region attaches the donor nucleic acid to the leading strand, thereby ligating the donor nucleic acid to the target nucleic acid. Further provided herein are methods, wherein the targeting strand dissociates from the complementary strand. Further provided herein are methods, wherein the new nucleic acid is incorporated into the target nucleic acid by hybridizing to the complementary strand.

Provided herein are methods, wherein the methods comprise: administering to a subject, an organ, a tissue, or a cell the system provided herein, the composition provided herein, or the cell provided herein, wherein the administering modifies a gene in the subject, the organ, the tissue, or the cell. Further provided here are methods, wherein the alteration comprises: an insertion, a deletion, a substitution, a change in copy number, a point mutation, a frameshift mutation, a missense mutation, a nonsense mutation, a mutation in a stop codon, an epigenetic mark, or any combination thereof. Further provided herein are methods, wherein the administering is local or systemic. Further provided herein are methods, wherein the administering is intranasal administration, subcutaneous administration, intravenous administration, inhalation, intramuscular administration, intratumoral peritumoral administration, administration, intrathecal administration, vaginal administration, or intradermal administration. Further provided herein are methods, wherein the subject is a mammal. Further provided herein are methods, wherein the subject has, is suspected of having, or is diagnosed with a disease or a condition. Further provided herein are methods, wherein the disease or the condition comprises a genetic disease or condition. Further provided herein are methods, wherein the methods further comprise administering to the subject a therapeutic agent.

Provided herein are methods, wherein the methods comprise: contacting a cell or a population of cells with the system provided herein or the composition provided herein, thereby modifying a gene in the cell. Further provided herein are methods, wherein the contacting is performed in vitro, in vivo, or ex vivo. Further provided herein are methods, wherein the alteration in the gene comprises: an insertion, a deletion, a substitution, a change in copy number, a point mutation, a frameshift mutation, a missense mutation, a nonsense mutation, a mutation in a stop codon, an epigenetic mark, or any combination thereof. Further provided herein are methods, wherein the alteration in the gene restores expression of a wild-type protein that is encoded by the gene relative to a comparable cell or population of cells that were not contacted with the system or the composition. Further provided herein are methods, wherein the cell comprises a eukaryotic cell or a prokaryotic cell. Further provided herein are methods, wherein the cell comprises a mammalian cell. Further provided herein are methods, wherein the population of cells comprises a population of human leukocytes, a population of stem cells, or a population of bacteria.

Provided herein are populations of cells, wherein the populations of cells are made by the methods provided herein.

Provided herein are kits, wherein the kits comprise: a system provided herein, a composition provided herein, a vector provided herein, a polynucleotide provided herein, or a cell provided herein, packaging and materials therefor.

Provided herein are kits, wherein the kits comprise: a first container comprising: a donor nucleic acid, and a guide polynucleotide or a polynucleotide encoding the guide polynucleotide, wherein the guide polynucleotide comprises: (i) a targeting region that has complementarity to the target nucleic acid; (ii) a protein binding region, wherein the protein binding region comprises a secondary structure that binds to a nuclease or a nickase; (iii) a ligation splint 2 region, wherein the donor hybridizing region has complementarity to the donor nucleic acid and has complementarity to a target nucleic acid, and wherein the ligation splint 2 region comprises at least one mismatch nucleobase relative to the target nucleic acid; and (iv) a ligation splint 1 region, wherein the ligation splint 1 region comprises deoxyribonucleotides and ribonucleotides, and a second container comprising: an engineered polypeptide comprising a nickase operably linked to a DNA ligase. Further provided herein are kits, wherein the kits further comprise reagents for nucleic acid amplification, transcription, translation, or nucleic acid isolation. Further provided herein are kits, wherein the kits further comprise a reporter molecule.

Provided herein are scaffolds, wherein the scaffolds comprise: the system provided herein or the composition provided herein; and a solid surface, wherein the system or the composition are immobilized to the solid surface.

### EXAMPLES

### Example 1. Editing Double-Stranded Target with the DNA Ligase System

A DNA editing system comprising Cas9 nickase (H840A), DNA ligase, guide polynucleotide, and donor nucleic acid were made and the system was evaluated with different double-stranded DNA targets (double-stranded DNA substrate). In particular, double-stranded DNA substrates, guide polynucleotide, and the RNP consisting of Cas9 H840A nickase, DNA ligase, and guide polynucleotide were generated and evaluated as described below.

Double-stranded DNA substrate generation: 5′-FAM-labeled double-stranded DNA (dsDNA) was obtained by annealing two oligos, Oligo 1 and Oligo 2, at a 1:1 ratio. In summary, the oligos were resuspended in nuclease-free water to 100 μM concentration. Subsequently, 10 μL of 5′-FAM-labeled oligo (Oligo 1) and 10 μL of Oligo 2 oligo were mixed with 10 μL of NEBuffer r3.1 (10×) and 70 μL of nuclease-free water in PCR tubes and incubated at 95 degrees Celsius for 5 mins and slowly cooled down at room temperature. The annealed FAM-labelled dsDNA was made at a concentration of 10 μM, or 10 picomoles/μL. Oligo sequences are listed in Table 3.

In vitro guide polynucleotide generation: In summary, a PCR was performed (Q5® Hot Start High-Fidelity 2× Master Mix, NEB) using oligos (Oligo 3, Oligo 4, Oligo 5 and Oligo 6) to generate double-stranded DNA template containing the T7 promoter for in vitro transcription. DNA from the PCR reaction was purified using AMPure XP Beads (Beckman Coulter) and the concentration was measured using Nanodrop. 75 ng of DNA template was used for in vitro transcription reaction using HiScribe® T7 Quick High Yield RNA Synthesis Kit (NEB). The guide nucleic acid product was purified using the Monarch® RNA Cleanup Kit (NEB) and the concentration was measured using the Nanodrop (Thermo Fisher Scientific). Oligo sequences are listed in Table 3. In Table 3, A, G, C, T are deoxyribonucleotides (DNA) and rA, rG, rC, rU are ribonucleotides (RNA).

TABLE 3
Oligonucleotides.

| SEQ ID | | |
|---|---|---|
| NO: | Name | Sequence (Listed from 5′ to 3′) |
| 1 | Oligo 1 | /5′6- |
| | | FAM/GATCACTTAGAGCAATCGGCCCAGACTGAGCACGTGATGG |
| | | CAGAGTACTAGGAT |
| 2 | Oligo 2 | ATCCTAGTACTCTGCCATCACGTGCTCAGTCTGGGCCGATTGCTC |
| | | TAAGTGATC |
| 3 | Oligo 3 | GGATCCTAATACGACTCACTATAGGCCCAGACTGAGCACGTGAG |
| | | TTTTAGAGCTAGAA |
| 4 | Oligo 4 | GCACCGACTCGGTGCCACTTTTTCAAGTTGATAACGGACTAGCC |
| | | TTATTTTAACTTGCTATTTCTAGCTCTAAAAC |
| 5 | Oligo 5 | GGATCCTAATACGACTCACTATAG |
| 6 | Oligo 6 | AAAAGCACCGACTCGG |
| 7 | Oligo 7 | AAAAGAGCACGAGATTGCAGAGTACTGCACCGACTCGG |
| 8 | Oligo 8 | AAAAACTGAGCACGAGATTGCAGAGTACTGCACCGACTCGG |
| 9 | Oligo 9 | AAAAGAGCACGAGATTGCAGAGTACTAGATGACGTAGCACCGA |
| | | CTCGG |
| 10 | Oligo 10 | AAAAACTGAGCACGAGATTGCAGAGTACTAGATGACGTAGCACC |
| | | GACTCGG |
| 11 | Oligo 11 | /5Phos/AGATTGCAGAGTACT |
| 12 | Oligo 12 | /5Phos/AGATTGCAGAGTACTAGATGACGTA |
| 13 | Oligo 13 | /5Phos/AGATTGCAGAGTACTAGATGACGTACGCAG |
| 14 | Oligo 14 | /5Phos/AGATTGCAGAGTACTAGATGACGTACGCAGACCAAGAAC |
| | | CGCAAGATGCG |
| 15 | Oligo 15 | /5Phos/AGATTGCAGAGTACTAGATGACGTACGCAGACCAAGAAC |
| | | CGCAAGATGCGACGGTGTACAAGTAATTGTC |
| 16 | Oligo 16 | /5Phos/AGATTGCAGAGTACTAGATGACGTACGCAGACCAAGAAC |
| | | CGCAAGATGCGACGGTGTACAAGTAATTGTCAACAGACCATCGT |
| | | GTTTTCA |
| 17 | Oligo 17 | AGTACTCTGCAATCTCGTGCATTT |
| 18 | Oligo 18 | AGTACTCTGCAATCTCGTGCTTTTT |
| 19 | Oligo 19 | AGTACTCTGCAATCTCGTGCTCTTTT |
| 20 | Oligo 20 | AGTACTCTGCAATCTCGTGCTCATTTT |
| 21 | Oligo 21 | AGTACTCTGCAATCTCGTGCTCAGATTT |
| 22 | Oligo 22 | AGTACTCTGCAATCTCGTGCTCAGTTTTT |
| 23 | Oligo 23 | CTGCGTACGTCATCTAGTACTCTGCAATCTCGTGCATTT |
| 24 | Oligo 24 | CGCATCTTGCGGTTCTTGGTCTGCGTACGTCATCTAGTACTCTGC |
| | | AATCTCGTGCATTT |
| 25 | Oligo 25 | CTGCGTACGTCATCTAGTACTCTGCAATCTCGTGCTCTTTT |
| 26 | Oligo 26 | CGCATCTTGCGGTTCTTGGTCTGCGTACGTCATCTAGTACTCTGC |
| | | AATCTCGTGCTCTTT |
| 27 | Oligo 27 | AGTACTCTGCAATCTCGTGCrUrCTTTT |
| 28 | Oligo 28 | AGTACTCTGCAATCTCGTGrCrUrCTTTT |
| 29 | Oligo 29 | AGTACTCTGCAATCTCGTrGrCrUrCTTTT |
| 30 | Oligo 30 | AGTACTCTGCAATCTCGrUrGrCrUrCTTTT |
| 31 | Oligo 31 | AGTACTCTGCAATCTCrGrUrGrCrUrCTTTT |
| 32 | Oligo 32 | /56-FAM/GATCACTTAGAGCAATCGGCCCAGACTGAGCACG |
| 33 | Oligo 33 | TGATGGCAGAGTACTAGGAT |
| 34 | Oligo 34 | CTGCGTACGTCATCTAGTACTCTGCAATCTCGTGCTCATTTT |
| 35 | Oligo 35 | CTGCGTACGTCATCTAGTACTCTGCAATCTCGTGCTCAGATTT |
| 36 | Oligo 36 | CTGCGTACGTCATCTAGTACTCTGCAATCTCGTGCTCAGTTTTT |
| 37 | Oligo 37 | CTGCGTACGTCATCTAGTACTCTGCAATCTCGTGCTCAGTCATTT |
| 38 | Oligo 38 | CTGCGTACGTCATCTAGTACTCTGCAATCTCGTGCTCAGTCTTTTT |
| 39 | Oligo 39 | CTGCGTACGTCATCTAGTACTCTGCAATCTCGTGCTCAGTCTGTT |
| | | TT |
| 40 | Oligo 40 | /5Phos/GGATCGCAGAGGAAA |
| 41 | Oligo 41 | /5Phos/GGATCGCAGAGGAAAGGAAGCCCTGCTTCC |
| 42 | Oligo 42 | AGTACTCTGCAATCTrCrGrUrGrCrUrCTTTT |
| 43 | Oligo 43 | AGTACTCTGCAATCTCGTGCTrCTTTT |
| 44 | Oligo 44 | AGTACTCTGCAATCTCGTGCTCTTTT |

Synthetic guide polynucleotide generation: All the synthetic guides listed in Table 4 were designed and were chemically synthesized. The guides were HPLC purified, and the mass of individual guide polynucleotides were verified by mass-spectrometry (LC-MS). In Table 4, A, C, G, U are ribonucleotides (RNA) and dA, dC, dG, dT are deoxyribonucleotides (DNA).

TABLE 4
Synthetic guide polynucleotides.

| SEQ ID NO: | Name | |
|---|---|---|
| 45 | Guide 1 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGdTdGdCU |
| 46 | Guide 2 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGdTdGdCdTU |
| 47 | Guide 3 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGdTdGdCdTdCU |
| 48 | Guide 4 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGdTdGdCUCU |
| 49 | Guide 5 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGdTdGCUCU |
| 50 | Guide 6 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGdTGCUCU |
| 51 | Guide 7 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCdGUGCUCU |
| 52 | Guide 8 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCdCGUGCUCU |
| 53 | Guide 9 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdTdTdCdCdTdCdTdGdCdG |
| | | dAdTdCdCCGUGCUCU |
| 54 | Guide 10 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdGdGdAdAdGdCdAdGdGdGd |
| | | CdTdTdCdCdTdTdTdCdCdTdCdTdGdCdGdAdTdCdCCGUGCU |
| | | CU |
| 55 | Guide 11 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCUUUCCUCUGCGAUCCCGU |
| | | GCUCU |
| 56 | Guide 12 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCdTdCdTdGdCdGdAdTdCdCC |
| | | GUGCUCUUUU |
| 57 | Guide 13 | GGCCCAGACUGAGCACGUGAGUUUUAGAGCUAGAAAUA |
| | | GCAAGUUAAAAUAAGGCUAGUCCGUUAUCAACUUGAAA |
| | | AAGUGGCACCGAGUCGGUGCUCUGCGdAdTdCdCdCdGdTd |
| | | GCUCUUUU |

In vitro DNA nicking, DNA ligation by DNA ligase, and analysis: First, CRISPR ribonucleoprotein (RNP) were formulated by combining 61 picomoles S.p. Cas9 H840A Nickase with 80 picomoles of guide RNA and incubated at room temperature for 10 mins to allow RNP complexation. Cas9 Nickase and DNA ligase activities were performed in a total of 7 μL volume containing 1 μL of FAM dsDNA (10 picomoles), 100 picomoles oligo for trans DNA editing (for cis DNA writing, the DNA template was incorporated into the guide polynucleotide sequence), 0.7 μL of 10×T4 DNA Ligase Reaction Buffer (NEB), 0.5 μL of DNA ligase, 2 μL of Cas9 Nickase-guide RNP and remaining volume of nuclease-free water to make a final volume of 7 μL. ligation donor oligos of various length were provided in trans at 100 picomoles per reaction. The reaction was incubated at 37 degrees Celsius for 1 hour and samples were treated with 0.5 μL of proteinase K solution (20 mg/mL, Qiagen) and incubated at 56 degrees Celsius for 30 min. Samples were heat inactivated at 95 degrees Celsius for 10 min, reaction products were combined with Gel Loading Buffer II (2×, ThermoFisher Scientific) and denatured at 95 degrees Celsius for 5 min, and separated by denaturing polyacrylamide gel (15% TBE-urea, 60 degrees Celsius, 150V) for 1 hour. DNA products were visualized by FAM fluorescence signal using a Life Technologies Gel Imaging system.

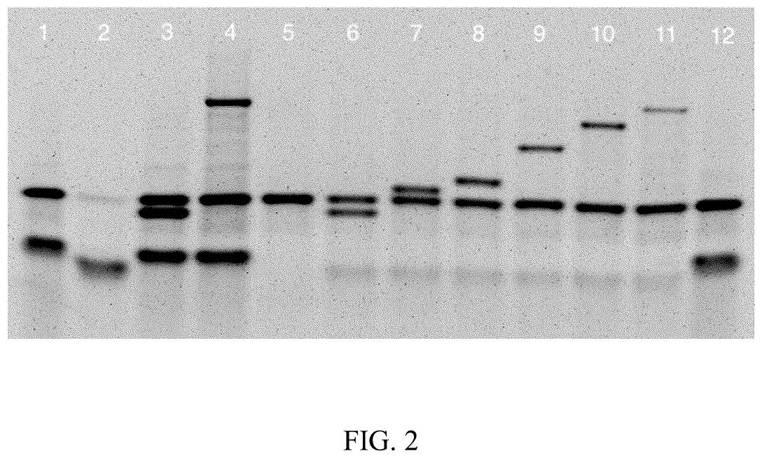

The editing products of targeted DNA editing by DNA ligase were analyzed by a denaturing urea polyacrylamide gel as shown in FIG. 2. Lanes 6-11 show the system successfully inserted nucleotides at different length, including 15 nt (lane 6, bottom band), 25 nt (lane 7, top band), 30 nt (lane 8, top band), 50 nt (lane 9, top band), 70 nt (lane 10, top band), and 90 nt (lane 11, top band).

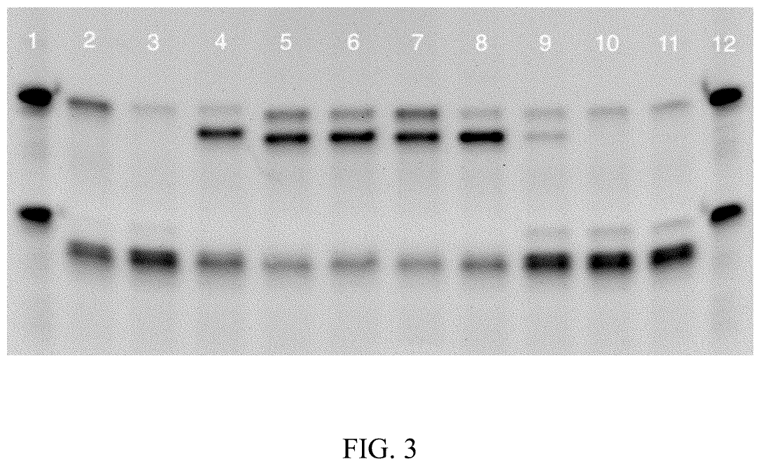

To determine the effects of the compositions of LS1 on the editing efficiency of the DNA editing system, the activity of DNA ligation editing by DNA ligase with the DNA ligation splint (LS) incorporated into different synthetic guide polynucleotide (Guide 1 through Guide 9 (SEQ ID NOs: 45-53)) was analyzed with a denaturing urea polyacrylamide gel as shown in FIG. 3. In particular Guide 1 through Guide 9 all have ligation splint 2 (LS2) with the following architecture: 5′ DDDDDDDDDDDDDDD 3′ (D represents DNA), while the LS1 consists of either all DNA bases (Guide 1, Guide 2, and Guide 3 (SEQ ID NOs: 45-47)) or both RNA and DNA (Guide 4, Guide 5, and Guide 6, Guide 7, Guide 8, and Guide 9 (SEQ ID NOs: 48-53)). The LS1 in Guide 1, Guide 2, and Guide 3 have the following architecture: 5′ DDDDD 3′, 5′ DDDDDD 3′, and 5′ DDDDDDD 3′, respectively (D represents DNA and R represents RNA). The LS1 in Guide 4, Guide 5, and Guide 6, Guide 7, Guide 8, and Guide 9 have the following architecture: 5′ DDDDDRR 3′, 5′ DDDDRRR 3′, 5′ DDDRRRR 3′, 5′ DDRRRRR 3′, 5′ DRRRRRR 3′, and 5′ RRRRRRR 3′, respectively. Lanes 3-11 have a ligation donor oligonucleotide (Oligo 40) that adds 15 nt to the nicked DNA resulting in an edited product of 49 nt.

FIG. 3 shows that majority of nicked product was converted to ligation edited product. However, increasing the length of LS1 further maximizes the efficiency. T4 DNA ligase activity for editing significantly dropped off when the number of DNA in LS1 is less than 3, suggesting that a minimum of 3 DNA nucleotides are necessary in the LS1 for effective DNA editing by T4 DNA ligase.

### Example 2. Plasmid Cloning

A backbone plasmid containing T7 RNA polymerase promoter, 5′ UTR sequence, and 3′ UTR sequence was constructed to clone various editor constructs. Gene fragments containing various Cas enzymes and DNA ligases were assembled utilizing ligation-based cloning or Gibson assembly into the backbone plasmid to serve as a template for in vitro transcription. All plasmids were midi or maxi prepped with the Qiagen Midi/Maxi Plus kits. The amino acid sequences of the engineered protein constructs that were generated are provided in Table 5 Below.

TABLE 5
Engineered Protein Constructs (DNA Ligase Genome Editors).

| SEQ ID | | | |
|---|---|---|---|
| NO: | Name | Description | Sequence |
| 89 | Construct 1 | SpCas9 H840A/Chlorella | MKRTADGSEFESPKKKRKVDKKYSIGLDIGTNSVG |
| | | DNA ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSAITKPLLAATLENIEDVQFPCLATPKIDGIR |
| | | | SVKQTQMLSRTFKPIRNSVMNRLLTELLPEGSDGEIS |
| | | | IEGATFQDTTSAVMTGHKMYNAKFSYYWFDYVTD |
| | | | DPLKKYIDRVEDMKNYITVHPHILEHAQVKIIPLIPV |
| | | | EINNITELLQYERDVLSKGFEGVMIRKPDGKYKFGR |
| | | | STLKEGILLKMKQFKDAEATIISMTALFKNTNTKTK |
| | | | DNFGYSKRSTHKSGKVEEDVMGSIEVDYDGVVFSI |
| | | | GTGFDADQRRDFWQNKESYIGKMVKFKYFEMGSK |
| | | | DCPRFPVFIGIRHEEDRSGGSKRTADGSEFEPKKKRK |
| | | | V |
| 90 | Construct 2 | SpCas9 H840A/Chlorella | MKRTADGSEFESPKKKRKVDKKYSIGLDIGTNSVG |
| | | DNA ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLINLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSTIAKPLLAATLENLDDVKFPCLVTPKIDGIR |
| | | | SLKQQHMLSRTFKPIRNSVMNKLLSELLPEGADGEI |
| | | | CIEDSTFQATTSAVMTGHKVYDEKFSYYWFDYVVD |
| | | | DPLKSYTDRVNDMKKYVDDHPHILEHEQVKIIPLIP |
| | | | VEINNIDELSQYERDVLAKGFEGVMIRRPDGKYKFG |
| | | | RSTLKEGILLKMKQFKDAEATIISMSPRLKNTNAKSK |
| | | | DNLGYSKRSTHKSGKVEEETMGSIEVDYDGVVFSIG |
| | | | TGFDDEQRKHFWENKDSYIGKLLKFKYFEMGSKDA |
| | | | PRFPVFIGIRHEEDCSGGSKRTADGSEFEPKKKRKV |
| 91 | Construct 3 | SpCas9 H840A/T3 DNA | MKRTADGSEFESPKKKRKVDKKYSIGLDIGTNSVG |
| | | Ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSNIFNTNPFKAVSFVESAVKKALETSGYLIA |
| | | | DCKYDGVRGNIVVDNVAEAAWLSRVSKFIPALEHL |
| | | | NGFDKRWQQLLNDDRCIFPDGFMLDGELMVKGVD |
| | | | FNTGSGLLRTKWVKRDNMGFHLTNVPTKLTPKGRE |
| | | | VIDGKFEFHLDPKRLSVRLYAVMPIHIAESGEDYDVQ |
| | | | NLLMPYHVEAMRSLLVEYFPEIEWLIAETYEVYDM |
| | | | DSLTELYEEKRAEGHEGLIVKDPQGIYKRGKKSGW |
| | | | WKLKPECEADGIIQGVNWGTEGLANEGKVIGFSVL |
| | | | LETGRLVDANNISRALMDEFTSNVKAHGEDFYNGW |
| | | | ACQVNYMEATPDGSLRHPSFEKFRGTEDNPQEKMS |
| | | | GGSKRTADGSEFEPKKKRKV |
| 92 | Construct 4 | SpCas9 H840A/T4 DNA | MKRTADGSEFESPKKKRKVDKKYSIGLDIGTNSVG |
| | | Ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSILKILNEIASIGSTKQKQAILEKNKDNELLK |
| | | | RVYRLTYSRGLQYYIKKWPKPGIATQSFGMLTLTDM |
| | | | LDFIEFTLATRKLTGNAAIEELTGYITDGKKDDVEVL |
| | | | RRVMMRDLECGASVSIANKVWPGLIPEQPQMLASS |
| | | | YDEKGINKNIKFPAFAQLKADGARCFAEVRGDELDD |
| | | | VRLLSRAGNEYLGLDLLKEELIKMTAEARQIHPEGV |
| | | | LIDGELVYHEQVKKEPEGLDFLFDAYPENSKAKEFA |
| | | | EVAESRTASNGIANKSLKGTISEKEAQCMKFQVWDY |
| | | | VPLVEIYSLPAFRLKYDVRFSKLEQMTSGYDKVILIE |
| | | | NQVVNNLDEAKVIYKKYIDQGLEGIILKNIDGLWEN |
| | | | ARSKNLYKFKEVIDVDLKIVGIYPHRKDPTKAGGFIL |
| | | | ESECGKIKVNAGSGLKDKAGVKSHELDRTRIMENQ |
| | | | NYYIGKILECECNGWLKSDGRTDYVKLFLPIAIRLRE |
| | | | DKTKANTFEDVFGDFHEVTGLSGGSKRTADGSEFEP |
| | | | KKKRKV |
| 93 | Construct 5 | SpCas9 H840A/T7 DNA | MKRTADGSEFESPKKKRKVDKKYSIGLDIGTNSVG |
| | | Ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSMNIKTNPFKAVSFVESAIKKALDNAGYLIA |
| | | | EIKYDGVRGNICVDNTANSYWLSRVSKTIPALEHLN |
| | | | GFDVRWKRLLNDDRCFYKDGFMLDGELMVKGVDF |
| | | | NTGSGLLRTKWTDTKNQEFHEELFVEPIRKKDKVPF |
| | | | KLHTGHLHIKLYAILPLHIVESGEDCDVMTLLMQEH |
| | | | VKNMLPLLQEYFPEIEWQAAESYEVYDMVELQQLY |
| | | | EQKRAEGHEGLIVKDPMCIYKRGKKSGWWKMKPE |
| | | | NEADGIIQGLVWGTKGLANEGKVIGFEVLLESGRLV |
| | | | NATNISRALMDEFTETVKEATLSQWGFFSPYGIGDN |
| | | | DACTINPYDGWACQISYMEETPDGSLRHPSFVMFRG |
| | | | TEDNPQEKMSGGSKRTADGSEFEPKKKRKV |
| 94 | Construct 6 | SpCas9 H840A/E. coli | MKRTADGSEFESPKKKRKVDKKYSIGLDIGTNSVG |
| | | DNA Ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSESIEQQLTELRTTLRHHEYLYHVMDAPEIP |
| | | | DAEYDRLMRELRELETKHPELITPDSPTQRVGAAPL |
| | | | AAFSQIRHEVPMLSLDNVFDEESFLAFNKRVQDRLK |
| | | | NNEKVTWCCELKLDGLAVSILYENGVLVSAATRGD |
| | | | GTTGEDITSNVRTIRAIPLKLHGENIPARLEVRGEVFL |
| | | | PQAGFEKINEDARRTGGKVFANPRNAAAGSLRQLD |
| | | | PRITAKRPLTFFCYGVGVLEGGELPDTHLGRLLQFK |
| | | | KWGLPVSDRVTLCESAEEVLAFYHKVEEDRPTLGF |
| | | | DIDGVVIKVNSLAQQEQLGFVARAPRWAVAFKFPAQ |
| | | | EQMTFVRDVEFQVGRTGAITPVARLEPVHVAGVLVS |
| | | | NATLHNADEIERLGLRIGDKVVIRRAGDVIPQVVNV |
| | | | VLSERPEDTREVVFPTHCPVCGSDVERVEGEAVARC |
| | | | TGGLICGAQRKESLKHFVSRRAMDVDGMGDKIIDQ |
| | | | LVEKEYVHTPADLFKLTAGKLTGLERMGPKSAQNV |
| | | | VNALEKAKETTFARFLYALGIREVGEATAAGLAAYF |
| | | | GTLEALEAASIEELQKVPDVGIVVASHVHNFFAEESN |
| | | | RNVISELLAEGVHWPAPIVINAEEIDSPFAGKTVVLT |
| | | | GSLSQMSRDDAKARLVELGAKVAGSVSKKTDLVIA |
| | | | GEAAGSKLAKAQELGIEVIDEAEMLRLLGSSGGSKR |
| | | | TADGSEFEPKKKRKV |
| 95 | Construct 7 | SpCas9 | MKRTADGSEFESPKKKRKVDKKYSIGLDIGTNSVG |
| | | H840A/Haemophilus | WAVITDEYKVPSKKFKVLGNTDRHSIKKNLIGALLF |
| | | Influenzae DNA Ligase | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSKFYRTLLLFFASSFAFANSDLMLLHTYNNQ |
| | | | PIEGWVMSEKLDGVRGYWNGKQLLTRQGQRLSPPA |
| | | | YFIKDFPPFAIDGELFSERNHFEEISTITKSFKGDGWE |
| | | | KLKLYVFDVPDAEGNLFERLAKLKAHLLEHPTTYIE |
| | | | IIEQIPVKDKTHLYQFLAQVENLQGEGVVVRNPNAP |
| | | | YERKRSSQILKLKTARGEECTVIAHHKGKGQFENV |
| | | | MGALTCKNHRGEFKIGSGFNLNERENPPPIGSVITYK |
| | | | YRGITNSGKPRFATYWREKKSGGSKRTADGSEFEPK |
| | | | KKRKV |
| 96 | Construct 8 | SpCas9 H840A/Human | MKRTADGSEFESPKKKRKVDKKYSIGLDIGTNSVG |
| | | DNA Ligase IV | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSAASQTSQTVASHVPFADLCSTLERIQKSKG |
| | | | RAEKIRHFREFLDSWRKFHDALHKNHKDVTDSFYP |
| | | | AMRLILPQLERERMAYGIKETMLAKLYIELLNLPRD |
| | | | GKDALKLLNYRTPTGTHGDAGDFAMIAYFVLKPRC |
| | | | LQKGSLTIQQVNDLLDSIASNNSAKRKDLIKKSLLQL |
| | | | ITQSSALEQKWLIRMIIKDLKLGVSQQTIFSVFHNDA |
| | | | AELHNVTTDLEKVCRQLHDPSVGLSDISITLFSAFKP |
| | | | MLAAIADIEHIEKDMKHQSFYIETKLDGERMQMHK |
| | | | DGDVYKYFSRNGYNYTDQFGASPTEGSLTPFIHNAF |
| | | | KADIQICILDGEMMAYNPNTQTFMQKGTKFDIKRM |
| | | | VEDSDLQTCYCVFDVLMVNNKKLGHETLRKRYEIL |
| | | | SSIFTPIPGRIEIVQKTQAHTKNEVIDALNEAIDKREE |
| | | | GIMVKQPLSIYKPDKRGEGWLKIKPEYVSGLMDEL |
| | | | DILIVGGYWGKGSRGGMMSHFLCAVAEKPPPGEKPS |
| | | | VFHTLSRVGSGCTMKELYDLGLKLAKYWKPFHRKA |
| | | | PPSSILCGTEKPEVYIEPCNSVIVQIKAAEIVPSDMYK |
| | | | TGCTLRFPRIEKIRDDKEWHECMTLDDLEQLRGKAS |
| | | | GKLASKHLYIGGDDEPQEKKRKAAPKMKKVIGIIEH |
| | | | LKAPNLTNVNKISNIFEDVEFCVMSGTDSQPKPDLE |
| | | | NRIAEFGGYIVQNPGPDTYCVIAGSENIRVKNIILSNK |
| | | | HDVVKPAWLLECFKTKSFVPWQPRFMIHMCPSTKE |
| | | | HFAREYDCYGDSYFIDTDLNQLKEVFSGIKNSNEQT |
| | | | PEEMASLIADLEYRYSWDCSPLSMFRRHTVYLDSYA |
| | | | VINDLSTKNEGTRLAIKALELRFHGAKVVSCLAEGV |
| | | | SHVIIGEDHSRVADFKAFRRTFKRKFKILKESWVTDS |
| | | | IDKCELQEENQYLISGGSKRTADGSEFEPKKKRKV |
| 97 | Construct 9 | SpCas9 H840A/Human | MKRTADGSEFESPKKKRKVDKKYSIGLDIGTNSVG |
| | | DNA Ligase I | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRRLENLIAQLPGEKKNGL |
| | | | FGNLIALSLGLTPNFKSNFDLAEDAKLQLSKDTYDD |
| | | | DLDNLLAQIGDQYADLFLAAKNLSDAILLSDILRVN |
| | | | TEITKAPLSASMIKRYDEHHQDLTLLKALVRQQLPE |
| | | | KYKEIFFDQSKNGYAGYIDGGASQEEFYKFIKPILEK |
| | | | MDGTEELLVKLNREDLLRKQRTFDNGSIPHQIHLGE |
| | | | LHAILRRQEDFYPFLKDNREKIEKILTFRIPYYVGPLA |
| | | | RGNSRFAWMTRKSEETITPWNFEEVVDKGASAQSFI |
| | | | ERMTNFDKNLPNEKVLPKHSLLYEYFTVYNELTKV |
| | | | KYVTEGMRKPAFLSGEQKKAIVDLLFKTNRKVTVK |
| | | | QLKEDYFKKIECFDSVEISGVEDRFNASLGTYHDLL |
| | | | KIIKDKDFLDNEENEDILEDIVLTLTLFEDREMIEERL |
| | | | KTYAHLFDDKVMKQLKRRRYTGWGRLSRKLINGIR |
| | | | DKQSGKTILDFLKSDGFANRNFMQLIHDDSLTFKEDI |
| | | | QKAQVSGQGDSLHEHIANLAGSPAIKKGILQTVKVV |
| | | | DELVKVMGRHKPENIVIEMARENQTTQKGQKNSRE |
| | | | RMKRIEEGIKELGSQILKEHPVENTQLQNEKLYLYYL |
| | | | QNGRDMYVDQELDINRLSDYDVDAIVPQSFLKDDSI |
| | | | DNKVLTRSDKNRGKSDNVPSEEVVKKMKNYWRQL |
| | | | LNAKLITQRKFDNLTKAERGGLSELDKAGFIKRQLV |
| | | | ETRQITKHVAQILDSRMNTKYDENDKLIREVKVITL |
| | | | KSKLVSDFRKDFQFYKVREINNYHHAHDAYLNAVV |
| | | | GTALIKKYPKLESEFVYGDYKVYDVRKMIAKSEQEI |
| | | | GKATAKYFFYSNIMNFFKTEITLANGEIRKRPLIETN |
| | | | GETGEIVWDKGRDFATVRKVLSMPQVNIVKKTEVQ |
| | | | TGGFSKESILPKRNSDKLIARKKDWDPKKYGGFDSP |
| | | | TVAYSVLVVAKVEKGKSKKLKSVKELLGITIMERSSF |
| | | | EKNPIDFLEAKGYKEVKKDLIIKLPKYSLFELENGRK |
| | | | RMLASAGELQKGNELALPSKYVNFLYLASHYEKLK |
| | | | GSPEDNEQKQLFVEQHKHYLDEIIEQISEFSKRVILA |
| | | | DANLDKVLSAYNKHRDKPIREQAENIIHLFTLTNLG |
| | | | APAAFKYFDTTIDRKRYTSTKEVLDATLIHQSITGLY |
| | | | ETRIDLSQLGGDSGGSSGGSSGSETPGTSESATPESSG |
| | | | GSSGGSSQRSIMSFFHPKKEGKAKKPEKEASNSSRE |
| | | | TEPPPKAALKEWNGVVSESDSPVKRPGRKAARVLG |
| | | | SEGEEEDEALSPAKGQKPALDCSQVSPPRPATSPENN |
| | | | ASLSDTSPMDSSPSGIPKRRTARKQLPKRTIQEVLEE |
| | | | QSEDEDREAKRKKEEEEEETPKESLTEAEVATEKEGE |
| | | | DGDQPTTPPKPLKTSKAETPTESVSEPEVATKQELQE |
| | | | EEEQTKPPRRAPKTLSSFFTPRKPAVKKEVKEEEPGA |
| | | | PGKEGAAEGPLDPSGYNPAKNNYHPVEDACWKPG |
| | | | QKVPYLAVARTFEKIEEVSARLRMVETLSNLLRSVV |
| | | | ALSPPDLLPVLYLSLNHLGPPQQGLELGVGDGVLLK |
| | | | AVAQATGRQLESVRAEAAEKGDVGLVAENSRSTQR |
| | | | LMLPPPPLTASGVFSKFRDIARLTGSASTAKKIDIIKG |
| | | | LFVACRHSEARFIARSLSGRLRLGLAEQSVLAALSQ |
| | | | AVSLTPPGQEFPPAMVDAGKGKTAEARKTWLEEQG |
| | | | MILKQTFCEVPDLDRIIPVLLEHGLERLPEHCKLSPGI |
| | | | PLKPMLAHPTRGISEVLKRFEEAAFTCEYKYDGQRA |
| | | | QIHALEGGEVKIFSRNQEDNTGKYPDIISRIPKIKLPS |
| | | | VTSFILDTEAVAWDREKKQIQPFQVLTTRKRKEVDA |
| | | | SEIQVQVCLYAFDLIYLNGESLVREPLSRRRQLLREN |
| | | | FVETEGEFVFATSLDTKDIEQIAEFLEQSVKDSCEGL |
| | | | MVKTLDVDATYEIAKRSHNWLKLKKDYLDGVGDT |
| | | | LDLVVIGAYLGRGKRAGRYGGFLLASYDEDSEELQ |
| | | | AICKLGTGFSDEELEEHHQSLKALVLPSPRPYVRIDG |
| | | | AVIPDHWLDPSAVWEVKCADLSLSPIYPAARGLVDS |
| | | | DKGISLRFPRFIRVREDKQPEQATTSAQVACLYRKQS |
| | | | QIQNQQGEDSGSDPEDTYSGGSKRTADGSEFEPKKK |
| | | | RKV |
| 98 | Construct | SpCas9 R221K, N394K, | MKRTADGSEFESPKKKRKVDKKYSIGLDIGTNSVG |
| | 10 | H840A/Chlorella DNA | WAVITDEYKVPSKKFKVLGNTDRHSIKKNLIGALLF |
| | | Ligase | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRKLENLIAQLPGEKKNG |
| | | | LFGNLIALSLGLTPNFKSNFDLAEDAKLQLSKDTYD |
| | | | DDLDNLLAQIGDQYADLFLAAKNLSDAILLSDILRV |
| | | | NTEITKAPLSASMIKRYDEHHQDLTLLKALVRQQLP |
| | | | EKYKEIFFDQSKNGYAGYIDGGASQEEFYKFIKPILE |
| | | | KMDGTEELLVKLKREDLLRKQRTFDNGSIPHQIHLG |
| | | | ELHAILRRQEDFYPFLKDNREKIEKILTFRIPYYVGPL |
| | | | ARGNSRFAWMTRKSEETITPWNFEEVVDKGASAQS |
| | | | FIERMTNFDKNLPNEKVLPKHSLLYEYFTVYNELTK |
| | | | VKYVTEGMRKPAFLSGEQKKAIVDLLFKTNRKVTV |
| | | | KQLKEDYFKKIECFDSVEISGVEDRFNASLGTYHDL |
| | | | LKIIKDKDFLDNEENEDILEDIVLTLTLFEDREMIEER |
| | | | LKTYAHLFDDKVMKQLKRRRYTGWGRLSRKLINGI |
| | | | RDKQSGKTILDFLKSDGFANRNFMQLIHDDSLTFKE |
| | | | DIQKAQVSGQGDSLHEHIANLAGSPAIKKGILQTVK |
| | | | VVDELVKVMGRHKPENIVIEMARENQTTQKGQKNS |
| | | | RERMKRIEEGIKELGSQILKEHPVENTQLQNEKLYLY |
| | | | YLQNGRDMYVDQELDINRLSDYDVDAIVPQSFLKD |
| | | | DSIDNKVLTRSDKNRGKSDNVPSEEVVKKMKNYW |
| | | | RQLLNAKLITQRKFDNLTKAERGGLSELDKAGFIKR |
| | | | QLVETRQITKHVAQILDSRMNTKYDENDKLIREVKV |
| | | | ITLKSKLVSDFRKDFQFYKVREINNYHHAHDAYLNA |
| | | | VVGTALIKKYPKLESEFVYGDYKVYDVRKMIAKSE |
| | | | QEIGKATAKYFFYSNIMNFFKTEITLANGEIRKRPLIE |
| | | | TNGETGEIVWDKGRDFATVRKVLSMPQVNIVKKTE |
| | | | VQTGGFSKESILPKRNSDKLIARKKDWDPKKYGGF |
| | | | DSPTVAYSVLVVAKVEKGKSKKLKSVKELLGITIME |
| | | | RSSFEKNPIDFLEAKGYKEVKKDLIIKLPKYSLFELE |
| | | | NGRKRMLASAGELQKGNELALPSKYVNFLYLASHY |
| | | | EKLKGSPEDNEQKQLFVEQHKHYLDEIIEQISEFSKR |
| | | | VILADANLDKVLSAYNKHRDKPIREQAENIIHLFTLT |
| | | | NLGAPAAFKYFDTTIDRKRYTSTKEVLDATLIHQSIT |
| | | | GLYETRIDLSQLGGDSGGSSGGSKRTADGSEFESPKK |
| | | | KRKVSGGSSGGSAITKPLLAATLENIEDVQFPCLATP |
| | | | KIDGIRSVKQTQMLSRTFKPIRNSVMNRLLTELLPEG |
| | | | SDGEISIEGATFQDTTSAVMTGHKMYNAKFSYYWF |
| | | | DYVTDDPLKKYIDRVEDMKNYITVHPHILEHAQVKI |
| | | | IPLIPVEINNITELLQYERDVLSKGFEGVMIRKPDGK |
| | | | YKFGRSTLKEGILLKMKQFKDAEATIISMTALFKNT |
| | | | NTKTKDNFGYSKRSTHKSGKVEEDVMGSIEVDYDG |
| | | | VVFSIGTGFDADQRRDFWQNKESYIGKMVKFKYFE |
| | | | MGSKDCPRFPVFIGIRHEEDRSGGSKRTADGSEFESP |
| | | | KKKRKVGSGPAAKRVKLD |
| 99 | Construct | SpCas9 R221K, N394K, | MKRTADGSEFESPKKKRKVDKKYSIGLDIGTNSVG |
| | 11 | H840A/T4 DNA Ligase | WAVITDEYKVPSKKFKVLGNTDRHSIKKNLIGALLF |
| | | | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRKLENLIAQLPGEKKNG |
| | | | LFGNLIALSLGLTPNFKSNFDLAEDAKLQLSKDTYD |
| | | | DDLDNLLAQIGDQYADLFLAAKNLSDAILLSDILRV |
| | | | NTEITKAPLSASMIKRYDEHHQDLTLLKALVRQQLP |
| | | | EKYKEIFFDQSKNGYAGYIDGGASQEEFYKFIKPILE |
| | | | KMDGTEELLVKLKREDLLRKQRTFDNGSIPHQIHLG |
| | | | ELHAILRRQEDFYPFLKDNREKIEKILTFRIPYYVGPL |
| | | | ARGNSRFAWMTRKSEETITPWNFEEVVDKGASAQS |
| | | | FIERMTNFDKNLPNEKVLPKHSLLYEYFTVYNELTK |
| | | | VKYVTEGMRKPAFLSGEQKKAIVDLLFKTNRKVTV |
| | | | KQLKEDYFKKIECFDSVEISGVEDRFNASLGTYHDL |
| | | | LKIIKDKDFLDNEENEDILEDIVLTLTLFEDREMIEER |
| | | | LKTYAHLFDDKVMKQLKRRRYTGWGRLSRKLINGI |
| | | | RDKQSGKTILDFLKSDGFANRNFMQLIHDDSLTFKE |
| | | | DIQKAQVSGQGDSLHEHIANLAGSPAIKKGILQTVK |
| | | | VVDELVKVMGRHKPENIVIEMARENQTTQKGQKNS |
| | | | RERMKRIEEGIKELGSQILKEHPVENTQLQNEKLYLY |
| | | | YLQNGRDMYVDQELDINRLSDYDVDAIVPQSFLKD |
| | | | DSIDNKVLTRSDKNRGKSDNVPSEEVVKKMKNYW |
| | | | RQLLNAKLITQRKFDNLTKAERGGLSELDKAGFIKR |
| | | | QLVETRQITKHVAQILDSRMNTKYDENDKLIREVKV |
| | | | ITLKSKLVSDFRKDFQFYKVREINNYHHAHDAYLNA |
| | | | VVGTALIKKYPKLESEFVYGDYKVYDVRKMIAKSE |
| | | | QEIGKATAKYFFYSNIMNFFKTEITLANGEIRKRPLIE |
| | | | TNGETGEIVWDKGRDFATVRKVLSMPQVNIVKKTE |
| | | | VQTGGFSKESILPKRNSDKLIARKKDWDPKKYGGF |
| | | | DSPTVAYSVLVVAKVEKGKSKKLKSVKELLGITIME |
| | | | RSSFEKNPIDFLEAKGYKEVKKDLIIKLPKYSLFELE |
| | | | NGRKRMLASAGELQKGNELALPSKYVNFLYLASHY |
| | | | EKLKGSPEDNEQKQLFVEQHKHYLDEIIEQISEFSKR |
| | | | VILADANLDKVLSAYNKHRDKPIREQAENIIHLFTLT |
| | | | NLGAPAAFKYFDTTIDRKRYTSTKEVLDATLIHQSIT |
| | | | GLYETRIDLSQLGGDSGGSSGGSKRTADGSEFESPKK |
| | | | KRKVSGGSSGGSILKILNEIASIGSTKQKQAILEKNK |
| | | | DNELLKRVYRLTYSRGLQYYIKKWPKPGIATQSFGM |
| | | | LTLTDMLDFIEFTLATRKLTGNAAIEELTGYITDGKK |
| | | | DDVEVLRRVMMRDLECGASVSIANKVWPGLIPEQP |
| | | | QMLASSYDEKGINKNIKFPAFAQLKADGARCFAEVR |
| | | | GDELDDVRLLSRAGNEYLGLDLLKEELIKMTAEAR |
| | | | QIHPEGVLIDGELVYHEQVKKEPEGLDFLFDAYPENS |
| | | | KAKEFAEVAESRTASNGIANKSLKGTISEKEAQCMK |
| | | | FQVWDYVPLVEIYSLPAFRLKYDVRFSKLEQMTSGY |
| | | | DKVILIENQVVNNLDEAKVIYKKYIDQGLEGIILKNI |
| | | | DGLWENARSKNLYKFKEVIDVDLKIVGIYPHRKDPT |
| | | | KAGGFILESECGKIKVNAGSGLKDKAGVKSHELDRT |
| | | | RIMENQNYYIGKILECECNGWLKSDGRTDYVKLFLP |
| | | | IAIRLREDKTKANTFEDVFGDFHEVTGLSGGSKRTA |
| | | | DGSEFESPKKKRKVGSGPAAKRVKLD |
| 100 | Construct | SpCas9 R221K, N394K, | MKRTADGSEFESPKKKRKVDKKYSIGLDIGTNSVG |
| | 12 | H840A/Human DNA | WAVITDEYKVPSKKFKVLGNTDRHSIKKNLIGALLF |
| | | Ligase IV | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRKLENLIAQLPGEKKNG |
| | | | LFGNLIALSLGLTPNFKSNFDLAEDAKLQLSKDTYD |
| | | | DDLDNLLAQIGDQYADLFLAAKNLSDAILLSDILRV |
| | | | NTEITKAPLSASMIKRYDEHHQDLTLLKALVRQQLP |
| | | | EKYKEIFFDQSKNGYAGYIDGGASQEEFYKFIKPILE |
| | | | KMDGTEELLVKLKREDLLRKQRTFDNGSIPHQIHLG |
| | | | ELHAILRRQEDFYPFLKDNREKIEKILTFRIPYYVGPL |
| | | | ARGNSRFAWMTRKSEETITPWNFEEVVDKGASAQS |
| | | | FIERMTNFDKNLPNEKVLPKHSLLYEYFTVYNELTK |
| | | | VKYVTEGMRKPAFLSGEQKKAIVDLLFKTNRKVTV |
| | | | KQLKEDYFKKIECFDSVEISGVEDRFNASLGTYHDL |
| | | | LKIIKDKDFLDNEENEDILEDIVLTLTLFEDREMIEER |
| | | | LKTYAHLFDDKVMKQLKRRRYTGWGRLSRKLINGI |
| | | | RDKQSGKTILDFLKSDGFANRNFMQLIHDDSLTFKE |
| | | | DIQKAQVSGQGDSLHEHIANLAGSPAIKKGILQTVK |
| | | | VVDELVKVMGRHKPENIVIEMARENQTTQKGQKNS |
| | | | RERMKRIEEGIKELGSQILKEHPVENTQLQNEKLYLY |
| | | | YLQNGRDMYVDQELDINRLSDYDVDAIVPQSFLKD |
| | | | DSIDNKVLTRSDKNRGKSDNVPSEEVVKKMKNYW |
| | | | RQLLNAKLITQRKFDNLTKAERGGLSELDKAGFIKR |
| | | | QLVETRQITKHVAQILDSRMNTKYDENDKLIREVKV |
| | | | ITLKSKLVSDFRKDFQFYKVREINNYHHAHDAYLNA |
| | | | VVGTALIKKYPKLESEFVYGDYKVYDVRKMIAKSE |
| | | | QEIGKATAKYFFYSNIMNFFKTEITLANGEIRKRPLIE |
| | | | TNGETGEIVWDKGRDFATVRKVLSMPQVNIVKKTE |
| | | | VQTGGFSKESILPKRNSDKLIARKKDWDPKKYGGF |
| | | | DSPTVAYSVLVVAKVEKGKSKKLKSVKELLGITIME |
| | | | RSSFEKNPIDFLEAKGYKEVKKDLIIKLPKYSLFELE |
| | | | NGRKRMLASAGELQKGNELALPSKYVNFLYLASHY |
| | | | EKLKGSPEDNEQKQLFVEQHKHYLDEIIEQISEFSKR |
| | | | VILADANLDKVLSAYNKHRDKPIREQAENIIHLFTLT |
| | | | NLGAPAAFKYFDTTIDRKRYTSTKEVLDATLIHQSIT |
| | | | GLYETRIDLSQLGGDSGGSSGGSKRTADGSEFESPKK |
| | | | KRKVSGGSSGGSAASQTSQTVASHVPFADLCSTLERI |
| | | | QKSKGRAEKIRHFREFLDSWRKFHDALHKNHKDVT |
| | | | DSFYPAMRLILPQLERERMAYGIKETMLAKLYIELLN |
| | | | LPRDGKDALKLLNYRTPTGTHGDAGDFAMIAYFVL |
| | | | KPRCLQKGSLTIQQVNDLLDSIASNNSAKRKDLIKKS |
| | | | LLQLITQSSALEQKWLIRMIIKDLKLGVSQQTIFSVF |
| | | | HNDAAELHNVTTDLEKVCRQLHDPSVGLSDISITLF |
| | | | SAFKPMLAAIADIEHIEKDMKHQSFYIETKLDGERM |
| | | | QMHKDGDVYKYFSRNGYNYTDQFGASPTEGSLTPF |
| | | | IHNAFKADIQICILDGEMMAYNPNTQTFMQKGTKFD |
| | | | IKRMVEDSDLQTCYCVFDVLMVNNKKLGHETLRK |
| | | | RYEILSSIFTPIPGRIEIVQKTQAHTKNEVIDALNEAID |
| | | | KREEGIMVKQPLSIYKPDKRGEGWLKIKPEYVSGLM |
| | | | DELDILIVGGYWGKGSRGGMMSHFLCAVAEKPPPG |
| | | | EKPSVFHTLSRVGSGCTMKELYDLGLKLAKYWKPF |
| | | | HRKAPPSSILCGTEKPEVYIEPCNSVIVQIKAAEIVPS |
| | | | DMYKTGCTLRFPRIEKIRDDKEWHECMTLDDLEQL |
| | | | RGKASGKLASKHLYIGGDDEPQEKKRKAAPKMKK |
| | | | VIGIIEHLKAPNLTNVNKISNIFEDVEFCVMSGTDSQP |
| | | | KPDLENRIAEFGGYIVQNPGPDTYCVIAGSENIRVKN |
| | | | IILSNKHDVVKPAWLLECFKTKSFVPWQPRFMIHMC |
| | | | PSTKEHFAREYDCYGDSYFIDTDLNQLKEVFSGIKNS |
| | | | NEQTPEEMASLIADLEYRYSWDCSPLSMFRRHTVYL |
| | | | DSYAVINDLSTKNEGTRLAIKALELRFHGAKVVSCL |
| | | | AEGVSHVIIGEDHSRVADFKAFRRTFKRKFKILKESW |
| | | | VTDSIDKCELQEENQYLISGGSKRTADGSEFESPKKK |
| | | | RKVGSGPAAKRVKLD |
| 101 | Construct | SpCas9 R221K, N394K, | MKRTADGSEFESPKKKRKVDKKYSIGLDIGTNSVG |
| | 13 | H840A/Human DNA | WAVITDEYKVPSKKFKVLGNTDRHSIKKNLIGALLF |
| | | Ligase IV | DSGETAEATRLKRTARRRYTRRKNRICYLQEIFSNEM |
| | | | AKVDDSFFHRLEESFLVEEDKKHERHPIFGNIVDEVA |
| | | | YHEKYPTIYHLRKKLVDSTDKADLRLIYLALAHMIK |
| | | | FRGHFLIEGDLNPDNSDVDKLFIQLVQTYNQLFEENP |
| | | | INASGVDAKAILSARLSKSRKLENLIAQLPGEKKNG |
| | | | LFGNLIALSLGLTPNFKSNFDLAEDAKLQLSKDTYD |
| | | | DDLDNLLAQIGDQYADLFLAAKNLSDAILLSDILRV |
| | | | NTEITKAPLSASMIKRYDEHHQDLTLLKALVRQQLP |
| | | | EKYKEIFFDQSKNGYAGYIDGGASQEEFYKFIKPILE |
| | | | KMDGTEELLVKLKREDLLRKQRTFDNGSIPHQIHLG |
| | | | ELHAILRRQEDFYPFLKDNREKIEKILTFRIPYYVGPL |
| | | | ARGNSRFAWMTRKSEETITPWNFEEVVDKGASAQS |
| | | | FIERMTNFDKNLPNEKVLPKHSLLYEYFTVYNELTK |
| | | | VKYVTEGMRKPAFLSGEQKKAIVDLLFKTNRKVTV |
| | | | KQLKEDYFKKIECFDSVEISGVEDRFNASLGTYHDL |
| | | | LKIIKDKDFLDNEENEDILEDIVLTLTLFEDREMIEER |
| | | | LKTYAHLFDDKVMKQLKRRRYTGWGRLSRKLINGI |
| | | | RDKQSGKTILDFLKSDGFANRNFMQLIHDDSLTFKE |
| | | | DIQKAQVSGQGDSLHEHIANLAGSPAIKKGILQTVK |
| | | | VVDELVKVMGRHKPENIVIEMARENQTTQKGQKNS |
| | | | RERMKRIEEGIKELGSQILKEHPVENTQLQNEKLYLY |
| | | | YLQNGRDMYVDQELDINRLSDYDVDAIVPQSFLKD |
| | | | DSIDNKVLTRSDKNRGKSDNVPSEEVVKKMKNYW |
| | | | RQLLNAKLITQRKFDNLTKAERGGLSELDKAGFIKR |
| | | | QLVETRQITKHVAQILDSRMNTKYDENDKLIREVKV |
| | | | ITLKSKLVSDFRKDFQFYKVREINNYHHAHDAYLNA |
| | | | VVGTALIKKYPKLESEFVYGDYKVYDVRKMIAKSE |
| | | | QEIGKATAKYFFYSNIMNFFKTEITLANGEIRKRPLIE |
| | | | TNGETGEIVWDKGRDFATVRKVLSMPQVNIVKKTE |
| | | | VQTGGFSKESILPKRNSDKLIARKKDWDPKKYGGF |
| | | | DSPTVAYSVLVVAKVEKGKSKKLKSVKELLGITIME |
| | | | RSSFEKNPIDFLEAKGYKEVKKDLIIKLPKYSLFELE |
| | | | NGRKRMLASAGELQKGNELALPSKYVNFLYLASHY |
| | | | EKLKGSPEDNEQKQLFVEQHKHYLDEIIEQISEFSKR |
| | | | VILADANLDKVLSAYNKHRDKPIREQAENIIHLFTLT |
| | | | NLGAPAAFKYFDTTIDRKRYTSTKEVLDATLIHQSIT |
| | | | GLYETRIDLSQLGGDSGGSSGGSKRTADGSEFESPKK |
| | | | KRKVSGGSSGGSAASQTSQTVASHVPFADLCSTLERI |
| | | | QKSKGRAEKIRHFREFLDSWRKFHDALHKNHKDVT |
| | | | DSFYPAMRLILPQLERERMAYGIKETMLAKLYIELLN |
| | | | LPRDGKDALKLLNYRTPTGTHGDAGDFAMIAYFVL |
| | | | KPRCLQKGSLTIQQVNDLLDSIASNNSAKRKDLIKKS |
| | | | LLQLITQSSALEQKWLIRMIIKDLKLGVSQQTIFSVF |
| | | | HNDAAELHNVTTDLEKVCRQLHDPSVGLSDISITLF |
| | | | SAFKPMLAAIADIEHIEKDMKHQSFYIETKLDGERM |
| | | | QMHKDGDVYKYFSRNGYNYTDQFGASPTEGSLTPF |
| | | | IHNAFKADIQICILDGEMMAYNPNTQTFMQKGTKFD |
| | | | IKRMVEDSDLQTCYCVFDVLMVNNKKLGHETLRK |
| | | | RYEILSSIFTPIPGRIEIVQKTQAHTKNEVIDALNEAID |
| | | | KREEGIMVKQPLSIYKPDKRGEGWLKIKPEYVSGLM |
| | | | DELDILIVGGYWGKGSRGGMMSHFLCAVAEKPPPG |
| | | | EKPSVFHTLSRVGSGCTMKELYDLGLKLAKYWKPF |
| | | | HRKAPPSSILCGTEKPEVYIEPCNSVIVQIKAAEIVPS |
| | | | DMYKTGCTLRFPRIEKIRDDKEWHECMTLDDLEQL |
| | | | RGKASGKLASKHLYIGGDDSGGSKRTADGSEFESPK |
| | | | KKRKVGSGPAAKRVKLD |

### Example 3. HEK293T Culture and Electroporation

A frozen vial of HEK293T (ATCC CRL-3216) cell line was thawed at 37° C. and cultured in Dulbecco's Modified Eagle's Medium supplemented with 10% (v/v) fetal bovine serum (FBS) at 37° C. with 5% CO2. Cells were grown to 90% confluency before passaging. Cells were passaged 3 times before any experiments. Before electroporation, 48-well plates were coated with poly-D-lysine for 1 hour and subsequently washed 3 times with PBS. After PBS wash, 400 μL of complete media without any antibiotic was added to each well and incubated at 37° C. with 5% CO₂ before use. All electroporations were performed using the Neon NxT electroporation system. For electroporation, 1 μg of purified mRNA, 100 μmol of each legRNA and splint, 100 μmol of donor DNA and 100,000 cells were mixed in Buffer R and electroporated with 10-μl Neon tips. Electroporation settings of 1150V, 20 ms, and 2 pulses were used for all electroporations. Following electroporation, cells were added to a single well of a 48-well plate containing cell culture media and incubated at 37° C. with 5% CO2. Genomic DNA was extracted 48 hours post-electroporation for NGS analysis.

### Example 4. In Vitro Transcribed (IVT) mRNA

Plasmids containing the gene of interest were completely digested and linearized by BsmBI (New England Biolabs) before use in the in vitro transcription reaction (IVT). The IVT reactions were performed using the NEB HiScribe T7 High Yield RNA Synthesis kit (New England Biolabs). In summary, the IVT reactions were performed at 37° C. with the addition of CleanCap Reagent AG (Trilink Biotechnologies) with a 100% replacement of UTP by N1-methylpseudo-UTP (Trilink Biotechnologies). The IVT reactions were terminated after 2 hours. Following IVT, each reaction was incubated with DNase I (New England Biolabs) for 15 minutes. Afterwards, the RNA was purified using the Monarch RNA Cleanup kit (New England Biolabs).

### Example 5. Next Generation Sequencing (NGS) Library Prep and Analysis

PCR primers (see Table 7) containing Illumina-compatible adapter sequences were used to amplify specific genomic regions of interest. Following standard PCR protocol using Q5 Hot Start High-Fidelity 2× Master Mix (New England Biolabs), the resulting PCR products were cleaned up with 0.7× Ampure XP Beads (Beckman Coulter). Purified PCR products were sent for the AmpExpress service at Quintara Biosciences. Amplicon sequencing data was analyzed with CRISPResso. Editing efficiencies were calculated from the alleles frequency table file based on the number of sequencing reads containing the desired edit of interest relative to the total number of sequencing reads within a given sample.

### Example 6. Gene Editing in HEK293 Cells with DNA Ligase and Nickase Engineered Proteins

To evaluate gene editing systems comprising Cas9 nickases, DNA ligases, guide polynucleotide, and donor nucleic acid in human cells, mRNA encoding the engineered protein constructs in Table 5 were in vitro transcribed according to the methods in Example 4 and synthetic guide RNAs in Table 6 were manufactured by chemical synthesis. The mRNA, synthetic guide RNAs, and oligonucleotides were delivered to HEK293T cells as described in Example 3.

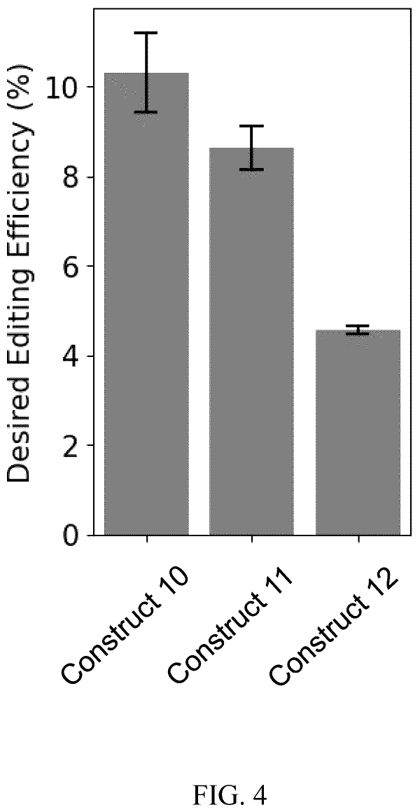

The engineered protein constructs (Constructs 1-13; SEQ ID NOS: 89-101) were screened for precision genome editing activity in HEK293T cells at genomic target site FANCF site 1. The results suggest DNA ligase genome editors facilitate precise genome editing in human cells. Of the thirteen engineered protein constructs, constructs containing T4 DNA ligase, Chlorella DNA ligase, and Human DNA Ligase IV editors enabled precise installation of a 3 bp substitution edit (+2 C>T; +4-5 TG>AC) at genome target FANCF site 1 in HEK293T cells (FIG. 4).

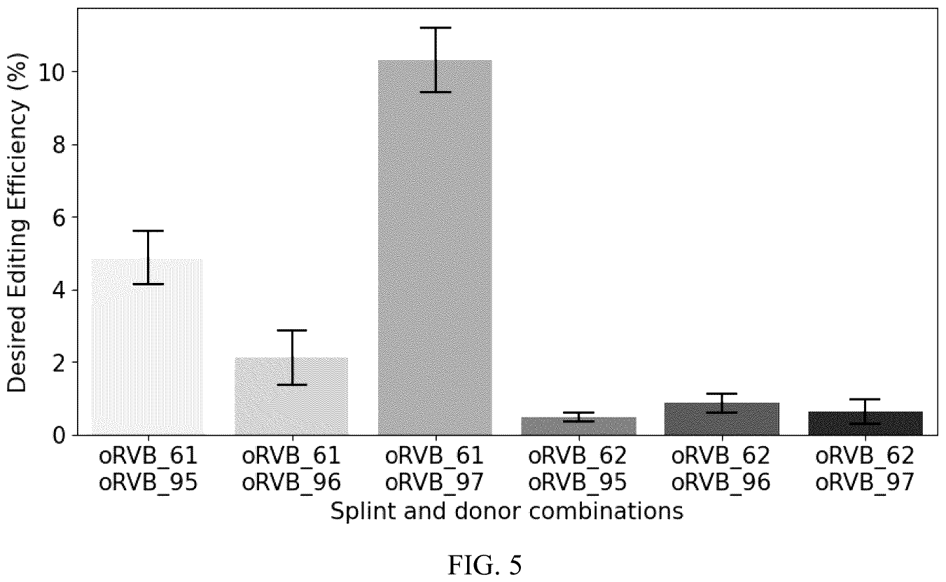

To verify the effects of splint and DNA donor modifications can enhance DNA ligase editing activity in human cells, various splint and DNA donor modifications (Table 7, SEQ ID NOS: 184-228) were tested for ligase editing utilizing the Chlorella DNA ligase editor construct (Construct 10, SEQ ID NO: 98) at the genomic target FANCF site 1 (gRVB_3) to install a 3 bp substitution edit (+2 C>T; +4-5 TG>AC). The editing efficiency of the gene editing system using various combinations of splint and DNA donors are shown in FIG. 5. The results suggest inclusion of modifications such as phosphorothioate bonds (PS) and locked nucleic acids (LNA) (as illustrated by the combination of oRVB_61 and oRVB_97) also improved ligase editing activity by up to 21.5-fold as compared to the combinations without such modifications (oRVB_62/oRVB_95).

To verify the effects of incorporation of RNA into guide RNA splint in DNA ligase editing, the gene editing systems containing the Chlorella DNA ligase editor construct (Construct 10) and various ligase editing guide RNAs were tested in HEK293T cells at HEK site 3 and AAV site 1. Delivery of in vitro transcribed mRNA encoding the ligase editors, synthetic guide RNAs, DNA splints and donors to HEK293T cells was performed via electroporation as described in Example 4.

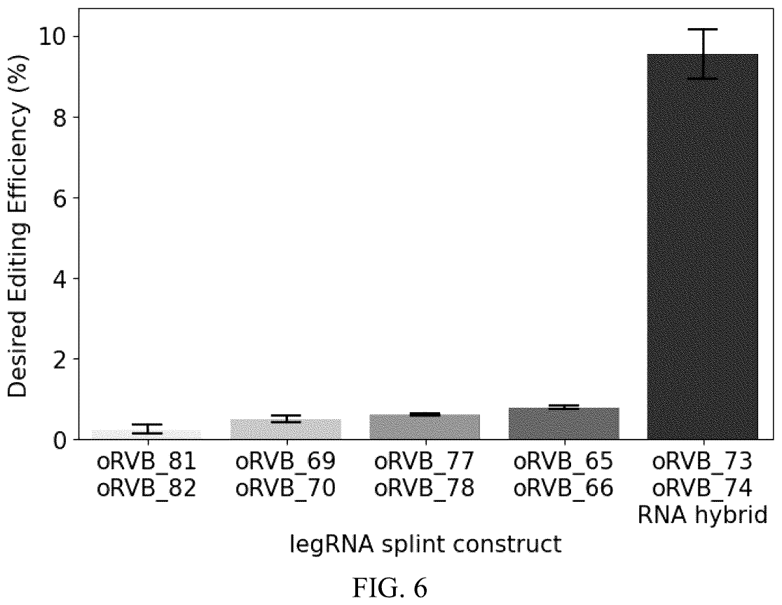

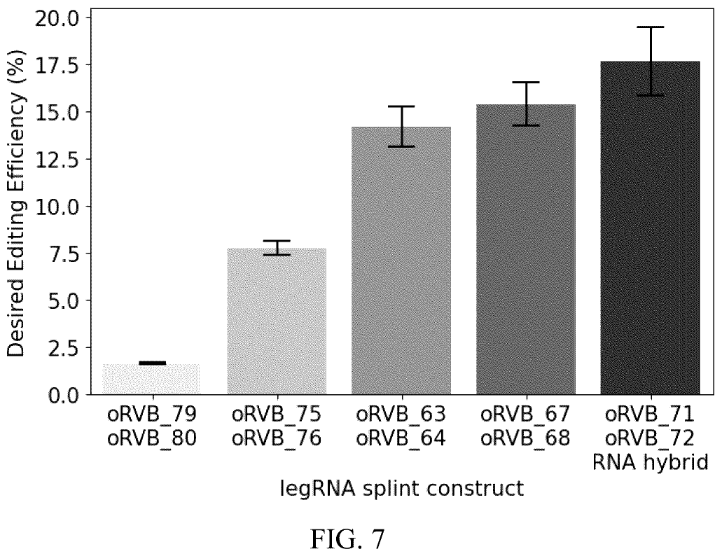

The mRNA was delivered to HEK293T cells as described in Example 3. As shown in FIG. 6, ligase editing guide RNAs (gRVB_5/gRVB_6) with inclusion of RNA within the splints (oRVB_73/oRVB_74) enhanced precise genome editing at HEK site 3 for the insertion of the attB site by up to 36.9-fold as compared to guide RNA splints without RNA incorporation (oRVB_81/oRVB_82). Similarly, as shown in FIG. 7, ligase editing guide RNAs (gRVB_1/gRVB_2) with inclusion of RNA within the splints (oRVB_71/oRVB_72) enhanced precise genome editing at AAV site 1 for the insertion of the attB site by up to 10.5-fold as compared to guide RNA splints without RNA incorporation (oRVB_79/oRVB_80).

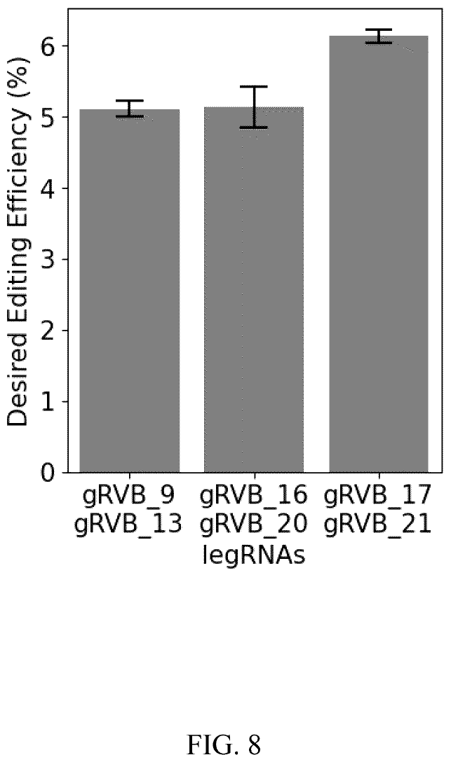

To verify the effects of the amount of RNA incorporated into the splint region of ligase editor guide RNAs (legRNAs), the gene editing system containing the Chlorella DNA ligase editor construct (Construct 10) and ligase editor guide RNAs (legRNAs) with splint region incorporated with various degrees of RNA were tested for ligase editing activity genomic target AAV site 1 in human cells. Desired editing efficiency refers to the efficiency of inserting the attB site at AAV site 1. Delivery of in vitro transcribed mRNA encoding the ligase editors, synthetic guide RNAs, and DNA donors to HEK293T cells was performed via electroporation as described in Example 4. As shown in FIG. 8, various degrees of RNA incorporation into the splint region of ligase editor guide RNAs (legRNAs) with single guide RNA architecture facilitated ligase editing activity at genomic target AAV site 1 in human cells.

TABLE 6
Synthetic guide polynucleotides.

| SEQ ID | | | |
|---|---|---|---|
| NO: | Name | Target | Sequence |
| 102 | gRVB_1 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCCAGCUGCGGUAUUGUGGmC |
| | | | *mG*mU |
| 103 | gRVB_2 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCCGUGGUUCCGGGCUGCAmU* |
| | | | mG*mA |
| 104 | gRVB_3 | FANCF site 1 | mG*mG*mA*AUCCCUUCUGCAGCACCGUUUUAGAGCUAGAA |
| | | | AUAGCAAGUUAAAAUAAGGCUAGUCCGUUAUCGACUUGAA |
| | | | AAAGUCGGACCGAGUCGGUCCAGCUGCGGUAUUGUGGmC* |
| | | | mG*mU |
| 105 | gRVB_4 | FANCF site 1 | mG*mG*mG*GUCCCAGGUGCUGACGUGUUUUAGAGCUAGA |
| | | | AAUAGCAAGUUAAAAUAAGGCUAGUCCGUUAUCGACUUGA |
| | | | AAAAGUCGGACCGAGUCGGUCCGUGGUUCCGGGCUGCAmU |
| | | | *mG*mA |
| 106 | gRVB_5 | HEK site 3 | mG*mG*mC*CCAGACUGAGCACGUGAGUUUUAGAGCUAGA |
| | | | AAUAGCAAGUUAAAAUAAGGCUAGUCCGUUAUCGACUUGA |
| | | | AAAAGUCGGACCGAGUCGGUCCAGCUGCGGUAUUGUGGmC |
| | | | *mG*mU |
| 107 | gRVB_6 | HEK site 3 | mG*mU*mC*AACCAGUAUCCCGGUGCGUUUUAGAGCUAGA |
| | | | AAUAGCAAGUUAAAAUAAGGCUAGUCCGUUAUCGACUUGA |
| | | | AAAAGUCGGACCGAGUCGGUCCGUGGUUCCGGGCUGCAmU |
| | | | *mG*mA |
| 108 | gRVB_7 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCCAGCUGCGGUAUUGUGGCG |
| | | | U+GdG+CdG+GdT+CdT+CdC+GdT+CdG+TdC+AdG+Gd |
| | | | A+TdC+AdTCCACUUCCAGG*mU*mU*mUU |
| 109 | gRVB_8 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCCAGCUGCGGUAUUGUGGCG |
| | | | U+GdG+CdG+GdT+CdT+CdC+GdT+CdG+TdC+AdG+GdA |
| | | | +TdC+AdTdCdCdACUUCCAGG*mU*mU*mUU |
| 110 | gRVB_9 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCCAGCUGCGGUAUUGUGGCG |
| | | | U+GdG+CdG+GdT+CdT+CdC+GdT+CdG+TdC+AdG+GdA |
| | | | +TdC+AdTdCdCdAdCdTUCCAGG*mU*mU*mUU |
| 111 | gRVB_10 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCCAGCUGCGGUAUUGUGGCG |
| | | | U+GdG+CdG+GdT+CdT+CdC+GdT+CdG+TdC+AdG+GdA |
| | | | +TdC+AdTdCdCdAdCdTdTdCdCdAdGdG*mU*mU*mUU |
| 112 | gRVB_11 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCCGUGGUUCCGGGCUGCAUGA |
| | | | +GdG+AdG+AdC+CdG+CdC+GdT+CdG+TdC+GdA+CdA+ |
| | | | AdG+CdCGGCGGUGGG*mU*mU*mUU |
| 113 | gRVB_12 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCCGUGGUUCCGGGCUGCAUGA |
| | | | +GdG+AdG+AdC+CdG+CdC+GdT+CdG+TdC+GdA+CdA |
| | | | +AdG+CdCdGdGdCGGUGGG*mU*mU*mUU |
| 114 | gRVB_13 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCCGUGGUUCCGGGCUGCAUGA |
| | | | +GdG+AdG+AdC+CdG+CdC+GdT+CdG+TdC+GdA+CdA |
| | | | +AdG+CdCdGdGdCdGdGUGGG*mU*mU*mUU |
| 115 | gRVB_14 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCCGUGGUUCCGGGCUGCAUGA |
| | | | +GdG+AdG+AdC+CdG+CdC+GdT+CdG+TdC+GdA+CdA |
| | | | +AdG+CdCdGdGdCdGdGdTdGdGdG*mU*mU*mUU |
| 116 | gRVB_15 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCC+GdG+CdG+GdT+CdT+CdC+ |
| | | | GdT+CdG+TdC+AdG+GdA+TdC+AdTCCACUUCCAGG*mU*mU* |
| | | | mUU |
| 117 | gRVB_16 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCC+GdG+CdG+GdT+CdT+CdC+ |
| | | | GdT+CdG+TdC+AdG+GdA+TdC+AdTdCdCdACUUCCAGG*mU* |
| | | | mU*mUU |
| 118 | gRVB_17 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCC+GdG+CdG+GdT+CdT+CdC+ |
| | | | GdT+CdG+TdC+AdG+GdA+TdC+AdTdCdCdAdCdTUCCAGG*mU |
| | | | *mU*mUU |
| 119 | gRVB_18 | AAV site 1 | mG*mC*mG*ACUCCUGGAAGUGGCCAGUUUUAGAGCUAGA |
| | | | AAUAGCAAGUUAAAAUAAGGCUAGUCCGUUAUCGACUUGA |
| | | | AAAAGUCGGACCGAGUCGGUCC+GdG+CdG+GdT+CdT+CdC+ |
| | | | GdT+CdG+TdC+AdG+GdA+TdC+AdTdCdCdAdCdTdTdCdCdAdGd |
| | | | G*mU*mU*mUU |
| 120 | gRVB_19 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCC+GdG+AdG+AdC+CdG+CdC+G |
| | | | dT+CdG+TdC+GdA+CdA+AdG+CdCGGCGGUGGG*mU*mU*mU |
| | | | U |
| 121 | gRVB_20 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCC+GdG+AdG+AdC+CdG+CdC+G |
| | | | dT+CdG+TdC+GdA+CdA+AdG+CdCdGdGdCGGUGGG*mU*mU* |
| | | | mUU |
| 122 | gRVB_21 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAA |
| | | | AAAGUCGGACCGAGUCGGUCC+GdG+AdG+AdC+CdG+CdC+G |
| | | | dT+CdG+TdC+GdA+CdA+AdG+CdCdGdGdCdGdGUGGG*mU |
| | | | *mU*mUU |
| 123 | gRVB_22 | AAV site 1 | mG*mC*mU*GGCCCCCCACCGCCCCAGUUUUAGAGCUAGAA |
| | | | AUAGCAAGUUAAAAUAAGGCUAGUCCGUUAUCGACUUGAAA |
| | | | AAGUCGGACCGAGUCGGUCC+GdG+AdG+AdC+CdG+CdC+ |
| | | | GdT+CdG+TdC+GdA+CdA+AdG+CdCdGdGdCdGdGdTd |
| | | | GdGdG*mU*mU*mUU |

TABLE 7
Oligonucleotides.

| SEQ ID | | | |
|---|---|---|---|
| NO | Name | Description | Oligo Sequence |
| 124 | oRVB_1 | FANCF site 1 | ACACTCTTTCCCTACACGACGCTCTTCCGATCTAACGGA |
| | | NGS primer F1 | TGGATGTGGCGCAGGTAG |
| 125 | oRVB_2 | FANCF site 1 | ACACTCTTTCCCTACACGACGCTCTTCCGATCTAAGTGA |
| | | NGS primer F2 | TGGATGTGGCGCAGGTAG |
| 126 | oRVB_3 | FANCF site 1 | ACACTCTTTCCCTACACGACGCTCTTCCGATCTAGCTGAT |
| | | NGS primer F3 | GGATGTGGCGCAGGTAG |
| 127 | oRVB_4 | FANCF site 1 | ACACTCTTTCCCTACACGACGCTCTTCCGATCTATAGGAT |
| | | NGS primer F4 | GGATGTGGCGCAGGTAG |
| 128 | oRVB_5 | FANCF site 1 | ACACTCTTTCCCTACACGACGCTCTTCCGATCTCGGAGA |
| | | NGS primer F5 | TGGATGTGGCGCAGGTAG |
| 129 | ORVB_6 | FANCF site 1 | ACACTCTTTCCCTACACGACGCTCTTCCGATCTCTTGGAT |
| | | NGS primer F6 | GGATGTGGCGCAGGTAG |
| 130 | oRVB_7 | FANCF site 1 | ACACTCTTTCCCTACACGACGCTCTTCCGATCTGAACGA |
| | | NGS primer F7 | TGGATGTGGCGCAGGTAG |
| 131 | oRVB_8 | FANCF site 1 | ACACTCTTTCCCTACACGACGCTCTTCCGATCTGAGTGA |
| | | NGS primer F8 | TGGATGTGGCGCAGGTAG |
| 132 | oRVB_9 | FANCF site 1 | ACACTCTTTCCCTACACGACGCTCTTCCGATCTGCAGGA |
| | | NGS primer F9 | TGGATGTGGCGCAGGTAG |
| 133 | oRVB_10 | FANCF site 1 | ACACTCTTTCCCTACACGACGCTCTTCCGATCTGTAAGA |
| | | NGS primer F10 | TGGATGTGGCGCAGGTAG |
| 134 | oRVB_11 | FANCF site 1 | ACACTCTTTCCCTACACGACGCTCTTCCGATCTTACAGAT |
| | | NGS primer F11 | GGATGTGGCGCAGGTAG |
| 135 | oRVB_12 | FANCF site 1 | ACACTCTTTCCCTACACGACGCTCTTCCGATCTTCTAGAT |
| | | NGS primer F12 | GGATGTGGCGCAGGTAG |
| 136 | oRVB_13 | FANCF site 1 | GACTGGAGTTCAGACGTGTGCTCTTCCGATCTACAGAGG |
| | | NGS primer R1 | CGTATCATTTCGCGGAT |
| 137 | oRVB_14 | FANCF site 1 | GACTGGAGTTCAGACGTGTGCTCTTCCGATCTAGAAAG |
| | | NGS primer R2 | GCGTATCATTTCGCGGAT |
| 138 | oRVB_15 | FANCF site 1 | GACTGGAGTTCAGACGTGTGCTCTTCCGATCTCTAAAGG |
| | | NGS primer R3 | CGTATCATTTCGCGGAT |
| 139 | oRVB_16 | FANCF site 1 | GACTGGAGTTCAGACGTGTGCTCTTCCGATCTCTGCAGG |
| | | NGS primer R4 | CGTATCATTTCGCGGAT |
| 140 | oRVB_17 | FANCF site 1 | GACTGGAGTTCAGACGTGTGCTCTTCCGATCTGCGGAG |
| | | NGS primer R5 | GCGTATCATTTCGCGGAT |
| 141 | oRVB_18 | FANCF site 1 | GACTGGAGTTCAGACGTGTGCTCTTCCGATCTGGATAGG |
| | | NGS primer R6 | CGTATCATTTCGCGGAT |
| 142 | oRVB_19 | FANCF site 1 | GACTGGAGTTCAGACGTGTGCTCTTCCGATCTTCAAAGG |
| | | NGS primer R7 | CGTATCATTTCGCGGAT |
| 143 | oRVB_20 | FANCF site 1 | GACTGGAGTTCAGACGTGTGCTCTTCCGATCTTTCCAGG |
| | | NGS primer R8 | CGTATCATTTCGCGGAT |
| 144 | oRVB_21 | HEK site 3 NGS | ACACTCTTTCCCTACACGACGCTCTTCCGATCTAACGGC |
| | | primer F1 | ATGGATGAGAGAAGCCTGGAG |
| 145 | oRVB_22 | HEK site 3 NGS | ACACTCTTTCCCTACACGACGCTCTTCCGATCTAAGTGC |
| | | primer F2 | ATGGATGAGAGAAGCCTGGAG |
| 146 | oRVB_23 | HEK site 3 NGS | ACACTCTTTCCCTACACGACGCTCTTCCGATCTAGCTGC |
| | | primer F3 | ATGGATGAGAGAAGCCTGGAG |
| 147 | oRVB_24 | HEK site 3 NGS | ACACTCTTTCCCTACACGACGCTCTTCCGATCTATAGGC |
| | | primer F4 | ATGGATGAGAGAAGCCTGGAG |
| 148 | oRVB_25 | HEK site 3 NGS | ACACTCTTTCCCTACACGACGCTCTTCCGATCTCGGAGC |
| | | primer F5 | ATGGATGAGAGAAGCCTGGAG |
| 149 | oRVB_26 | HEK site 3 NGS | ACACTCTTTCCCTACACGACGCTCTTCCGATCTCTTGGC |
| | | primer F6 | ATGGATGAGAGAAGCCTGGAG |
| 150 | oRVB_27 | HEK site 3 NGS | ACACTCTTTCCCTACACGACGCTCTTCCGATCTGAACGC |
| | | primer F7 | ATGGATGAGAGAAGCCTGGAG |
| 151 | oRVB_28 | HEK site 3 NGS | ACACTCTTTCCCTACACGACGCTCTTCCGATCTGAGTGC |
| | | primer F8 | ATGGATGAGAGAAGCCTGGAG |
| 152 | oRVB_29 | HEK site 3 NGS | ACACTCTTTCCCTACACGACGCTCTTCCGATCTGCAGGC |
| | | primer F9 | ATGGATGAGAGAAGCCTGGAG |
| 153 | oRVB_30 | HEK site 3 NGS | ACACTCTTTCCCTACACGACGCTCTTCCGATCTGTAAGC |
| | | primer F10 | ATGGATGAGAGAAGCCTGGAG |
| 154 | oRVB_31 | HEK site 3 NGS | ACACTCTTTCCCTACACGACGCTCTTCCGATCTTACAGC |
| | | primer F11 | ATGGATGAGAGAAGCCTGGAG |
| 155 | oRVB_32 | HEK site 3 NGS | ACACTCTTTCCCTACACGACGCTCTTCCGATCTTCTAGC |
| | | primer F12 | ATGGATGAGAGAAGCCTGGAG |
| 156 | oRVB_33 | HEK site 3 NGS | GACTGGAGTTCAGACGTGTGCTCTTCCGATCTACAGCCC |
| | | primer R1 | AGCCAAACTTGTCAACC |
| 157 | oRVB_34 | HEK site 3 NGS | GACTGGAGTTCAGACGTGTGCTCTTCCGATCTAGAACCC |
| | | primer R2 | AGCCAAACTTGTCAACC |
| 158 | oRVB_35 | HEK site 3 NGS | GACTGGAGTTCAGACGTGTGCTCTTCCGATCTCTAACCC |
| | | primer R3 | AGCCAAACTTGTCAACC |
| 159 | oRVB_36 | HEK site 3 NGS | GACTGGAGTTCAGACGTGTGCTCTTCCGATCTCTGCCCC |
| | | primer R4 | AGCCAAACTTGTCAACC |
| 160 | oRVB_37 | HEK site 3 NGS | GACTGGAGTTCAGACGTGTGCTCTTCCGATCTGCGGCCC |
| | | primer R5 | AGCCAAACTTGTCAACC |
| 161 | oRVB_38 | HEK site 3 NGS | GACTGGAGTTCAGACGTGTGCTCTTCCGATCTGGATCCC |
| | | primer R6 | AGCCAAACTTGTCAACC |
| 162 | oRVB_39 | HEK site 3 NGS | GACTGGAGTTCAGACGTGTGCTCTTCCGATCTTCAACCC |
| | | primer R7 | AGCCAAACTTGTCAACC |
| 163 | oRVB_40 | HEK site 3 NGS | GACTGGAGTTCAGACGTGTGCTCTTCCGATCTTTCCCCC |
| | | primer R8 | AGCCAAACTTGTCAACC |
| 164 | oRVB_41 | AAVS1 NGS | ACACTCTTTCCCTACACGACGCTCTTCCGATCTAACGCG |
| | | primer F1 | GGAACTGCCGCTGGC |
| 165 | oRVB_42 | AAVS1 NGS | ACACTCTTTCCCTACACGACGCTCTTCCGATCTAAGTCG |
| | | primer F2 | GGAACTGCCGCTGGC |
| 166 | oRVB_43 | AAVS1 NGS | ACACTCTTTCCCTACACGACGCTCTTCCGATCTAGCTCG |
| | | primer F3 | GGAACTGCCGCTGGC |
| 167 | oRVB_44 | AAVS1 NGS | ACACTCTTTCCCTACACGACGCTCTTCCGATCTATAGCG |
| | | primer F4 | GGAACTGCCGCTGGC |
| 168 | oRVB_45 | AAVS1 NGS | ACACTCTTTCCCTACACGACGCTCTTCCGATCTCGGACG |
| | | primer F5 | GGAACTGCCGCTGGC |
| 169 | oRVB_46 | AAVS1 NGS | ACACTCTTTCCCTACACGACGCTCTTCCGATCTCTTGCG |
| | | primer F6 | GGAACTGCCGCTGGC |
| 170 | oRVB_47 | AAVS1 NGS | ACACTCTTTCCCTACACGACGCTCTTCCGATCTGAACCG |
| | | primer F7 | GGAACTGCCGCTGGC |
| 171 | oRVB_48 | AAVS1 NGS | ACACTCTTTCCCTACACGACGCTCTTCCGATCTGAGTCG |
| | | primer F8 | GGAACTGCCGCTGGC |
| 172 | oRVB_49 | AAVS1 NGS | ACACTCTTTCCCTACACGACGCTCTTCCGATCTGCAGCG |
| | | primer F9 | GGAACTGCCGCTGGC |
| 173 | oRVB_50 | AAVS1 NGS | ACACTCTTTCCCTACACGACGCTCTTCCGATCTGTAACG |
| | | primer F10 | GGAACTGCCGCTGGC |
| 174 | oRVB_51 | AAVS1 NGS | ACACTCTTTCCCTACACGACGCTCTTCCGATCTTACACG |
| | | primer F11 | GGAACTGCCGCTGGC |
| 175 | oRVB_52 | AAVS1 NGS | ACACTCTTTCCCTACACGACGCTCTTCCGATCTTCTACG |
| | | primer F12 | GGAACTGCCGCTGGC |
| 176 | oRVB_53 | AAVS1 NGS | GACTGGAGTTCAGACGTGTGCTCTTCCGATCTACAGGAG |
| | | primer R1 | GAGGCCCTCATCTGGCG |
| 177 | oRVB_54 | AAVS1 NGS | GACTGGAGTTCAGACGTGTGCTCTTCCGATCTAGAAGA |
| | | primer R2 | GGAGGCCCTCATCTGGCG |
| 178 | oRVB_55 | AAVS1 NGS | GACTGGAGTTCAGACGTGTGCTCTTCCGATCTCTAAGAG |
| | | primer R3 | GAGGCCCTCATCTGGCG |
| 179 | oRVB_56 | AAVS1 NGS | GACTGGAGTTCAGACGTGTGCTCTTCCGATCTCTGCGAG |
| | | primer R4 | GAGGCCCTCATCTGGCG |
| 180 | oRVB_57 | AAVS1 NGS | GACTGGAGTTCAGACGTGTGCTCTTCCGATCTGCGGGA |
| | | primer R5 | GGAGGCCCTCATCTGGCG |
| 181 | oRVB_58 | AAVS1 NGS | GACTGGAGTTCAGACGTGTGCTCTTCCGATCTGGATGAG |
| | | primer R6 | GAGGCCCTCATCTGGCG |
| 182 | oRVB_59 | AAVS1 NGS | GACTGGAGTTCAGACGTGTGCTCTTCCGATCTTCAAGAG |
| | | primer R7 | GAGGCCCTCATCTGGCG |
| 183 | oRVB_60 | AAVS1 NGS | GACTGGAGTTCAGACGTGTGCTCTTCCGATCTTTCCGAG |
| | | primer R8 | GAGGCCCTCATCTGGCG |
| 184 | oRVB_61 | FANCF site 1 | +G*A*+AG+CT+CG+GA+AA+AG+CG+AT+CG+TG+ATGCT |
| | | ligation splint | GCAGAAGGACGCCA+CA+AT+AC+CG+CA+G*C*+T |
| 185 | oRVB_62 | FANCF site 1 | G*A*AGCTCGGAAAAGCGATCGTGATGCTGCAGAAGGA |
| | | ligation splint | CGCCACAATACCGCAG*C*T |
| 186 | oRVB_63 | AAV site 1 | +G*G*+CG+GT+CT+CC+GT+CG+TC+AG+GA+TC+ATCCA |
| | | ligation splint | CTTCCAGGACGCCA+CA+AT+AC+CG+CA+G*C*+T |
| 187 | oRVB_64 | AAV site 1 | +G*G*+AG+AC+CG+CC+GT+CG+TC+GA+CA+AG+CCGG |
| | | ligation splint | CGGTGGGTCATGC+AG+CC+CG+GA+AC+C*A*+C |
| 188 | oRVB_65 | HEK site 3 | +G*G*+AG+AC+CG+CC+GT+CG+TC+GA+CA+AG+CCCG |
| | | ligation splint | TGCTCAGTCACGCCA+CA+AT+AC+CG+CA+G*C*+T |
| 189 | oRVB_66 | HEK site 3 | +G*G*+CG+GT+CT+CC+GT+CG+TC+AG+GA+TC+ATCCG |
| | | ligation splint | GGATACTGTCATGC+AG+CC+CG+GA+AC+C*A*+C |
| 190 | oRVB_67 | AAV site 1 | +G*G*+CG+GT+CT+CC+GT+CG+TC+AG+GA+TC+ATCCA |
| | | ligation splint | CTTCCAGGTCATGC+AG+CC+CG+GA+AC+C*A*+C |
| 191 | oRVB_68 | AAV site 1 | +G*G*+AG+AC+CG+CC+GT+CG+TC+GA+CA+AG+CCGG |
| | | ligation splint | CGGTGGGACGCCA+CA+AT+AC+CG+CA+G*C*+T |
| 192 | oRVB_69 | HEK site 3 | +G*G*+AG+AC+CG+CC+GT+CG+TC+GA+CA+AG+CCCG |
| | | ligation splint | TGCTCAGTCTCATGC+AG+CC+CG+GA+AC+C*A*+C |
| 193 | oRVB_70 | HEK site 3 | +G*G*+CG+GT+CT+CC+GT+CG+TC+AG+GA+TC+ATCCG |
| | | ligation splint | GGATACTGACGCCA+CA+AT+AC+CG+CA+G*C*+T |
| 194 | oRVB_71 | AAV site 1 | +G*G*+CG+GT+CT+CC+GT+CG+TC+AG+GA+TC+ATCCA |
| | | ligation splint | CTrUrCrCrArGrGACGCCA+CA+AT+AC+CG+CA+G*C*+T |
| 195 | oRVB_72 | AAV site 1 | +G*G*+AG+AC+CG+CC+GT+CG+TC+GA+CA+AG+CCGG |
| | | ligation splint | CGGrUrGrGrGTCATGC+AG+CC+CG+GA+AC+C*A*+C |
| 196 | oRVB_73 | HEK site 3 | +G*G*+AG+AC+CG+CC+GT+CG+TC+GA+CA+AG+CCCG |
| | | ligation splint | TGCrUrCrArGrUrCACGCCA+CA+AT+AC+CG+CA+G*C*+T |
| 197 | oRVB_74 | HEK site 3 | +G*G*+CG+GT+CT+CC+GT+CG+TC+AG+GA+TC+ATCCG |
| | | ligation splint | GGrArUrArCrUrGTCATGC+AG+CC+CG+GA+AC+C*A*+C |
| 198 | oRVB_75 | AAV site 1 | +G*G*+CG+GT+CT+CC+GT+CG+TC+AGGATCATCCACTT |
| | | ligation splint | CCAGGACGCCA+CA+AT+AC+CG+CA+G*C*+T |
| 199 | oRVB_76 | AAV site 1 | +G*G*+AG+AC+CG+CC+GT+CG+TC+GACAAGCCGGCGG |
| | | ligation splint | TGGGTCATGC+AG+CC+CG+GA+AC+C*A*+C |
| 200 | oRVB_77 | HEK site 3 | +G*G*+AG+AC+CG+CC+GT+CG+TC+GACAAGCCCGTGC |
| | | ligation splint | TCAGTCACGCCA+CA+AT+AC+CG+CA+G*C*+T |
| 201 | oRVB_78 | HEK site 3 | +G*G*+CG+GT+CT+CC+GT+CG+TC+AGGATCATCCGGGA |
| | | ligation splint | TACTGTCATGC+AG+CC+CG+GA+AC+C*A*+C |
| 202 | oRVB_79 | AAV site 1 | +G*G*+CG+GT+CT+CC+GT+CG+TC+AG+GA+TC+AT+CC |
| | | ligation splint | +AC+TT+CC+AG+GACGCCACAATACCGCAG*C*T |
| 203 | oRVB_80 | AAV site 1 | +G*G*+AG+AC+CG+CC+GT+CG+TC+GA+CA+AG+CC+G |
| | | ligation splint | G+CG+GT+GG+GTCATGCAGCCCGGAACC*A*C |
| 204 | oRVB_81 | HEK site 3 | +G*G*+AG+AC+CG+CC+GT+CG+TC+GA+CA+AG+CC+CG |
| | | ligation splint | +TG+CT+CA+GT+CACGCCACAATACCGCAG*C*T |
| 205 | oRVB_82 | HEK site 3 | +G*G*+CG+GT+CT+CC+GT+CG+TC+AG+GA+TC+AT+CC |
| | | ligation splint | +GG+GA+TA+CT+GTCATGCAGCCCGGAACC*A*C |
| 206 | oRVB_83 | AAV site 1 | +G*G*+CG+GT+CT+CC+GT+CG+TC+AG+GA+TC+ATrCrCr |
| | | ligation splint | ArCrUrUrCrCrArGrGACGCCA+CA+AT+AC+CG+CA+G*C*+ |
| | | | T |
| 207 | oRVB_84 | AAV site 1 | +G*G*+CG+GT+CT+CC+GT+CG+TC+AG+GA+TC+ATCCAr |
| | | ligation splint | CrUrUrCrCrArGrGACGCCA+CA+AT+AC+CG+CA+G*C*+T |
| 208 | oRVB_85 | AAV site 1 | +G*G*+CG+GT+CT+CC+GT+CG+TC+AG+GA+TC+ATCCA |
| | | ligation splint | CTTCrCrArGrGACGCCA+CA+AT+AC+CG+CA+G*C*+T |
| 209 | oRVB_86 | AAV site 1 | +G*G*+AG+AC+CG+CC+GT+CG+TC+GA+CA+AG+CCrGr |
| | | ligation splint | GrCrGrGrUrGrGrGTCATGC+AG+CC+CG+GA+AC+C*A*+C |
| 210 | oRVB_87 | AAV site 1 | +G*G*+AG+AC+CG+CC+GT+CG+TC+GA+CA+AG+CCGG |
| | | ligation splint | CrGrGrUrGrGrGTCATGC+AG+CC+CG+GA+AC+C*A*+C |
| 211 | oRVB_88 | AAV site 1 | +G*G*+AG+AC+CG+CC+GT+CG+TC+GA+CA+AG+CCGG |
| | | ligation splint | CGGTGrGrGTCATGC+AG+CC+CG+GA+AC+C*A*+C |
| 212 | oRVB_89 | AAV site 1 | +G*G*+AG+AC+CG+CC+GT+CG+TC+GA+CA+AG+CCrCr |
| | | ligation splint | GrUrGrCrUrCrArGrUrCACGCCA+CA+AT+AC+CG+CA+G*C |
| | | | *+T |
| 213 | oRVB_90 | AAV site 1 | +G*G*+AG+AC+CG+CC+GT+CG+TC+GA+CA+AG+CCCG |
| | | ligation splint | TrGrCrUrCrArGrUrCACGCCA+CA+AT+AC+CG+CA+G*C*+ |
| | | | T |
| 214 | oRVB_91 | AAV site 1 | +G*G*+AG+AC+CG+CC+GT+CG+TC+GA+CA+AG+CCCG |
| | | ligation splint | TGCTCrArGrUrCACGCCA+CA+AT+AC+CG+CA+G*C*+T |
| 215 | oRVB_92 | AAV site 1 | +G*G*+CG+GT+CT+CC+GT+CG+TC+AG+GA+TC+ATrCrCr |
| | | ligation splint | GrGrGrArUrArCrUrGTCATGC+AG+CC+CG+GA+AC+C*A*+ |
| | | | C |
| 216 | oRVB_93 | AAV site 1 | +G*G*+CG+GT+CT+CC+GT+CG+TC+AG+GA+TC+ATCCGr |
| | | ligation splint | GrGrArUrArCrUrGTCATGC+AG+CC+CG+GA+AC+C*A*+C |
| 217 | oRVB_94 | AAV site 1 | +G*G*+CG+GT+CT+CC+GT+CG+TC+AG+GA+TC+ATCCG |
| | | ligation splint | GGATrArCrUrGTCATGC+AG+CC+CG+GA+AC+C*A*+C |
| 218 | oRVB_1 | FANCF site 1 3 | /5Phos/ATCACGATCGCTTTTCCGAGCTTC |
| | | bp substitution | |
| | | donor 1 | |
| 219 | oRVB_2 | FANCF site 1 3 | /5Phos/A*T*CACGATCGCTTTTCCGAGCT*T*C |
| | | bp substitution | |
| | | donor 2 | |
| 220 | oRVB_3 | FANCF site 1 3 | /5Phos/ATCACGATCGCTTTTCCGAGCT*T*C |
| | | bp substitution | |
| | | donor 3 | |
| 221 | oRVB_4 | attB insertion | /5Phos/atgatcctgacgacggagaccgcc |
| | | donor 1.1 | |
| 222 | oRVB_5 | attB insertion | /5Phos/ggcttgtcgacgacggcggtctcc |
| | | donor 1.2 | |
| 223 | oRVB_6 | attB insertion | /5Phos/atgatcctgacgacggagaccg*c*c |
| | | donor 2.1 | |
| 224 | oRVB_7 | attB insertion | /5Phos/ggcttgtcgacgacggcggtct*c*c |
| | | donor 2.2 | |
| 225 | oRVB_8 | attB insertion | /5Phos/atgat/iMe-dC//iMe-dC/tga/iMe-dC/ga/iMe- |
| | | donor 3.1 | dC/ggaga/iMe-dC//iMe-dC/g/iMe-dC//3Me-dC/ |
| 226 | oRVB_9 | attB insertion | /5Phos/gg/iMe-dC/ttgt/iMe-dC/ga/iMe-dC/ga/ |
| | | donor 3.2 | iMe-dC/gg/iMe-dC/ggt/iMe-dC/t/iMe-dC//3Me-dC/ |
| 227 | oRVB_10 | attB insertion | /5Phos/atgat/iMe-dC//iMe-dC/tga/iMe-dC/ga/iMe- |
| | | donor 4.1 | dC/ggaga/iMe-dC//iMe-dC/g*/iMe-dC/*/3Me-dC/ |
| 228 | oRVB_11 | attB insertion | /5Phos/gg/iMe-dC/ttgt/iMe-dC/ga/iMe-dC/ga/iMe- |
| | | donor 4.2 | dC/gg/iMe-dC/ggt/iMe-dC/t*/iMe-dC/*/3Me-dC/ |

In Table 6 and Table 7, A, C, G, U are ribonucleotides (RNA) and dA, dC, dG, dT are deoxyribonucleotides (DNA), +A, +T, +C, +G are locked nucleic acids (LNA), rA, rU, rC, rG are ribonucleotides (RNA), * signifies a Phosphorothioate (PS) bond, mA, mU, mC, mG correspond to 2′-O-Methyl RNA, /iMe-Dc/ corresponds to 5′-methyl dC, /3Me-dC/ corresponds to 3′ 5-methyl dC, and /5Phos/ corresponds to 5′ phosphorylation.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

What is claimed is:

1. A composition comprising:
(a) a donor nucleic acid;
(b) a polynucleotide encoding an engineered protein construct comprising a nickase or a variant thereof and a DNA ligase or a functional fragment thereof; and
(c) a guide polynucleotide, wherein the guide polynucleotide comprises:
(i) a targeting region that has complementarity to a target nucleic acid;
(ii) a protein binding region, wherein the protein binding region comprises a secondary structure that binds to the engineered protein construct comprising a nickase region;
(iii) a ligation splint 2 region, wherein the ligation splint region has complementarity to the donor nucleic acid and has complementarity to the target nucleic acid, and wherein the ligation splint region comprises at least one alteration relative to the target nucleic acid; and
(iv) a ligation splint 1 region, wherein the ligation splint 1 region comprises: a deoxyribonucleotide and a ribonucleotide, wherein the ligation splint 1 region has complementarity to the target nucleic acid.

2. The composition of claim 1, wherein the ligation splint 1 region forms a DNA-RNA (DR)-loop upon association with the target nucleic acid and a DNA ligase.

3. The composition of claim 2, wherein the ligation splint 1 region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18 or 20:1.

4. The composition of claim 1, wherein the ligation splint 2 region comprises at least about 5 nucleotides up to 10,000 nucleotides.

5. The composition of claim 1, wherein the ligation splint 1 region comprises at least about 5 nucleotides up to 20 nucleotides.

6. The composition of claim 1, wherein the ligation splint 2 region comprises a reverse complement sequence of
(1) a non-coding polynucleotide sequence or a variant thereof;
(2) a sequence encoding a coding region of a polynucleotide sequence or a variant thereof; or
(3) a sequence encoding for an exon or an intron.

7. The composition of claim 1, wherein the ligation splint 2 region comprises a complement sequence of a sequence encoding
(1) a non-coding polynucleotide sequence or a variant thereof;
(2) a coding region of a polynucleotide sequence or a variant thereof; or
(3) a sequence encoding for an exon or an intron.

8. The composition of claim 1, wherein the ligation splint 2 region comprises a sequence comprising at least one nucleobase that is complementary to or mismatched with a sequence encoding a splice acceptor site.

9. The composition of claim 1, wherein the nickase is an engineered Cas protein or a functional variant thereof comprising at least one amino acid substitution at position 840 corresponding to SEQ ID NO: 69.

10. The composition of claim 9, wherein the engineered Cas protein or a functional variant thereof further comprises at least one amino acid substitution at position 221, 394, or a combination thereof, corresponding to SEQ ID NO: 69.

11. The composition of claim 9, wherein the engineered Cas protein or a functional variant thereof comprises amino acid substitutions of R221K, N394K, H840A, or any combination thereof.

12. The composition of claim 9, wherein the engineered Cas protein or a functional variant thereof comprises a sequence 97%, 98%, 99%, or 100% identical to SEQ ID NO: 70 or SEQ ID NO: 71.

13. The composition of claim 1, wherein the DNA ligase or a functional fragment comprises an E. coli DNA ligase, a Taq DNA ligase, a T3 DNA ligase, a T4 DNA ligase, a T7 DNA ligase, a Chlorella virus DNA ligase, a human DNA ligase I, a human DNA ligase II, a human DNA ligase III, a human DNA ligase IV, a human ligase V, a variant, or a combination thereof.

14. The composition of claim 1, wherein the DNA ligase or a functional fragment thereof comprises a sequence 90%, 91%, 92%, 93%, 94%, 95%, 97%, 98%, 99%, or 100% identical to SEQ ID NOS: 58-66.

15. The composition of claim 1, wherein the DNA ligase or a functional fragment thereof is linked to the nickase or a variant thereof.

16. The composition of claim 1, wherein the engineered protein construct comprises a sequence 90%, 91%, 92%, 93%, 94%, 95%, 97%, 98%, 99%, or 100% identical to SEQ ID NOS: 89-101.

17. An engineered fusion protein comprising:
(a) a DNA ligase or a functional fragment thereof; and
(b) an engineered nickase that comprises three amino acid substitutions at positions corresponding to amino acid positions 221, 394, and 840 of a nuclease comprising a sequence of SEQ ID NO: 69.

18. A system for modifying a target nucleic acid, the system comprising:
(a) a donor nucleic acid;
(b) an engineered protein construct comprising a nickase region or a variant thereof and a DNA ligase or a functional fragment thereof, or a polynucleotide encoding the engineered protein construct; and
(c) a guide polynucleotide, wherein the guide polynucleotide comprises:
(i) a targeting region that has complementarity to a target nucleic acid;
(ii) a protein binding region, wherein the protein binding region comprises a secondary structure that binds to the engineered protein construct comprising a nickase region;
(iii) a ligation splint 2 region, wherein the ligation splint region has complementarity to the donor nucleic acid and has complementarity to the target nucleic acid, and wherein the ligation splint region comprises at least one alteration relative to the target nucleic acid; and
(iv) a ligation splint 1 region, wherein the ligation splint 1 region comprises: a deoxyribonucleotide and a ribonucleotide, wherein the ligation splint 1 region has complementarity to the target nucleic acid,
wherein upon introduction of the system to a cell, a nucleus, or a cell-free system, the system incorporates the donor nucleic acid into the target nucleic acid, thereby modifying the target nucleic acid.

19. A method of ligating a donor nucleic acid with a target nucleic acid, the method comprising:
contacting a cell or a cell-free system with:
(a) a donor nucleic acid
(b) a guide polynucleotide or a polynucleotide encoding the guide polynucleotide, wherein the guide polynucleotide comprises:
(i) a targeting region that has complementarity to a target nucleic acid;
(ii) a protein binding region, wherein the protein binding region comprises a secondary structure that binds to a nuclease; and
(iii) a ligation splint 2 region, wherein the ligation splint 2 region is complementary to the donor nucleic acid and has complementarity to the target nucleic acid and at least one alteration relative to the target nucleic acid;
(iv) a ligation splint 1 region, wherein the ligation splint 1 region comprises: a deoxyribonucleotide and a ribonucleotide;
(c) an engineered protein or a polynucleotide encoding the engineered protein, wherein the engineered protein comprises:
(i) a nuclease region; and
(ii) a DNA ligase region;
wherein:
the guide polynucleotide forms a complex with the engineered protein via the protein binding region,
the nuclease region of the engineered protein generates a break in the target nucleic acid to generate a leading strand and a complementary strand,
the targeting region forms a complex with the complementary strand,
the ligation splint 1 region forms a complex with the leading strand, and
wherein the DNA ligase region attaches the donor nucleic acid to the leading strand, thereby ligating the donor nucleic acid to the target nucleic acid.

20. The method of claim 19, wherein the targeting strand dissociates from the complementary strand.
